(19) United States
(12) Patent Application Publication
(10) Pub. No.: US 2003/0068320 A1
(43) Pub. Date: Apr. 10, 2003

(54) Methods of administering/dosing CD2 antagonists for the prevention and treatment of autoimmune disorders or inflammatory disorders

(76) Inventor: Christine Dingivan, Germantown, MD (US)

Correspondence Address: PENNIE AND EDMONDS, 1155 AVENUE OF THE AMERICAS, NEW YORK, NY, 100362711

(21) Appl. No.: 10/091,268

(22) Filed: Mar. 4, 2002

Related U.S. Application Data
(60) Provisional application No. 60/273,098, filed on Mar. 2, 2001.
(60) Provisional application No. 60/346,918, filed on Oct. 19, 2001.
(60) Provisional application No. 60/358,424, filed on Feb. 19, 2002.

(51) Int. Cl. A61K 39/395

(52) U.S. Cl. 424/144.1

(57) ABSTRACT

The present invention provides compositions for the prevention or treatment of an autoimmune disorder or an inflammatory disorder in a subject comprising one or more CD2 antagonists. In particular, the invention provides methods for preventing or treating an autoimmune disorder or an inflammatory disorder in a subject comprising administering one or more CD2 binding molecules to said subject. The present invention provides doses of CD2 binding molecules and methods of administration that result in improved efficacy, while avoiding or reducing the adverse or unwanted side effects associated with the administration of an agent that induces the depletion of peripheral blood lymphocytes.

This application is entitled to and claims priority benefit to U.S. provisional application Serial No. 60/273,098, filed Mar. 2, 2001, U.S. provisional application No. 60/346,918, filed Oct. 19, 2001, and U.S. provisional application Serial No. ______, filed Feb. 19, 2002, the contents of each of which is incorporated herein by reference in its entirety.

### 1. INTRODUCTION

The present invention relates to compositions comprising CD2 antagonists and methods for preventing, treating or ameliorating symptoms of an autoimmune disorder or an inflammatory disorder utilizing said compositions. In particular, the present invention relates to compositions comprising CD2 antagonists and methods for preventing, treating or ameliorating symptoms of an autoimmune disorder or an inflammatory disorder utilizing said compositions. The present invention provides methods of administering CD2 binding molecules that result in improved efficacy, while not compromising safety. The present invention also provides methods of preventing or treating autoimmune disorders or inflammatory disorders comprising administering doses of CD2 binding molecules that result in at least 25% of the CD2 polypeptides expressed by peripheral blood lymphocytes being bound by CD2 binding molecules and achieve a lymphocyte count between 500 cells/mm³ and 1200 cells/mm³. Further, the methods of the invention reduce or avoid the adverse side effects associated with the administration of immunosuppressive agents.

### 2. BACKGROUND OF THE INVENTION

2.1. Autoimmune Diseases

Autoimmune diseases are caused when the body's immune system, which is meant to defend the body against bacteria, viruses, and any other foreign product, malfunctions and produces antibodies against healthy tissue, cells and organs. Antibodies, T cells and macrophages provide beneficial protection, but can also produce harmful or deadly immunological responses.

The principle mechanisms by which auto-antibodies can produce an autoimmune disease are complement-dependent lytic destruction of the target cell, opsonization, formation of immune complexes, blockade of receptor sites for physiological ligands, and stimulation of cell surface receptors. The auto-antibody can bind to cell surface receptors and either inhibit or stimulate the specialized function of the cell (Paul, W. E. Ed., 1989, Fundamental Immunology, Raven Press, New York, Chapter31, p. 839).

Autoimmune diseases can be organ specific or systemic and are provoked by different pathogenic mechanisms. Organ specific autoimmunization is characterized by tolerance and suppression within the T cell compartment, aberrant expression of major-histocompatibility complex (MHC) antigens, antigenic mimicry and allelic variations in MHC genes. Systemic autoimmune diseases involve polyclonal B cell activation and abnormalities of immunoregulatory T cells, T cell receptors and MHC genes. Examples of organ specific autoimmune diseases are diabetes, hyperthyroidism, autoimmune adrenal insufficiency, pure red cell anemia, multiple sclerosis and rheumatic carditis. Representative systemic autoimmune diseases are systemic lupus erythematosus, rheumatoid arthritis, chronic inflammation, Sjogren's syndrome polymyositis, dermatomyositis and scleroderma.

Current treatment of autoimmune diseases involves administering immunosuppressive agents such as cortisone, aspirin derivatives, hydroxychloroquine, methotrexate, azathioprine and cyclophsophamide or combinations thereof. The dilemma faced when administering immunosuppressive agents, however, is the more effectively the autoimmune disease is treated, the more defenseless the patient is left to attack from infections.

2.2. Inflammatory Disorders

Inflammation is a process by which the body's white blood cells and chemicals protect our bodies from infection by foreign substances, such as bacteria and viruses. It is usually characterized by pain, swelling, warmth and redness of the affected area. Chemicals known as cytokines and prostaglandins control this process, and are released in an ordered and self-limiting cascade into the blood or affected tissues. This release of chemicals increases the blood flow to the area of injury or infection, and may result in the redness and warmth. Some of the chemicals cause a leak of fluid into the tissues, resulting in welling. This protective process may stimulate nerves and cause pain. These changes, when occurring for a limited period in the relevant area, work to the benefit of the body.

Rheumatoid arthritis (RA) and juvenile rheumatoid arthritis are types of inflammatory arthritis. Arthritis is a general term that describes inflammation in joints. Some, but not all, types of arthritis are the result of misdirected inflammation. Besides rheumatoid arthritis, other types of arthritis associated with inflammation include the following: psoriatic arthritis, Reiter's syndrome, ankylosing spondylitis arthritis, and gouty arthritis. Rheumatoid arthritis is a type of chronic arthritis that occurs in joints on both sides of the body (such as both hands, wrists or knees). This symmetry helps distinguish rheumatoid arthritis from other types of arthritis. In addition to affecting the joints, rheumatoid arthritis may occasionally affect the skin, eyes, lungs, heart, blood or nerves.

Rheumatoid arthritis affects about 1% of the world's population and istentially disabling. There are approximately 2.9 million incidences of rheumatoid arthritis in the United States. Two to three times more women are affected than men. The typical age that rheumatoid arthritis occurs is between 25 and 50. Juvenile rheumatoid arthritis affects 71,000 young Americans (aged eighteen and under), affecting six times as many girls as boys.

Rheumatoid arthritis is an autoimmune disorder where the body's immune system improperly identifies the synovial membranes that secrete the lubricating fluid in the joints as foreign. Inflammation results, and the cartilage and tissues in and around the joints are damaged or destroyed. In severe cases, this inflammation extends to other joint tissues and surrounding cartilage, where it may erode or destroy bone and cartilage and lead to joint deformities. The body replaces damaged tissue with scar tissue, causing the normal spaces within the joints to become narrow and the bones to fuse together. Rheumatoid arthritis creates stiffness, swelling, fatigue, anemia, weight loss, fever, and often, crippling pain. Some common symptoms of rheumatoid arthritis include joint stiffness upon awakening that lasts an hour or longer; swelling in a specific finger or wrist joints; swelling in the soft tissue around the joints; and swelling on both sides of the joint. Swelling can occur with or without pain, and can worsen progressively or remain the same for years before progressing. The diagnosis of rheumatoid arthritis is based on a combination of factors, including: the specific location and symmetry of painful joints, the presence of joint stiffness in the morning, the presence of bumps and nodules under the skin (rheumatoid nodules), results of X-ray tests that suggest rheumatoid arthritis, and/or positive results of a blood test called the rheumatoid factor. Many, but not all, people with rheumatoid arthritis have the rheumatoid-factor antibody in their blood. The rheumatoid factor may be present in people who do not have rheumatoid arthritis. Other diseases can also cause the rheumatoid factor to be produced in the blood. That is why the diagnosis of rheumatoid arthritis is based on a combination of several factors and not just the presence of the rheumatoid factor in the blood.

The typical course of the disease is one of persistent but fluctuating joint symptoms, and after about 10 years, 90% of sufferers will show structural damage to bone and cartilage. A small percentage will have a short illness that clears up completely, and another small percentage will have very severe disease with many joint deformities, and occasionally other manifestations of the disease. The inflammatory process causes erosion or destruction of bone and cartilage in the joints. In rheumatoid arthritis, there is an autoimmune cycle of persistent antigen presentation, T-cell stimulation, cytokine secretion, synovial cell activation, and joint destruction. The disease has a major impact on both the individual and society, causing significant pain, impaired function and disability, as well as costing millions of dollars in healthcare expenses and lost wages. (See, for example, the NIH website and the NIAID website).

Currently available therapy for arthritis focuses on reducing inflammation of the joints with anti-inflammatory or immunosuppressive medications. The first line of treatment of any arthritis is usually anti-inflammatories, such as aspirin, ibuprofen and Cox-2 inhibitors such as celecoxib and rofecoxib. “Second line drugs” include gold, methotrexate and steroids. Although these are well-established treatments for arthritis, very few patients remit on these lines of treatment alone. Recent advances in the understanding of the pathogenesis of rheumatoid arthritis have led to the use of methotrexate in combination with antibodies to cytokines or recombinant soluble receptors. For example, recombinant soluble receptors for tumor necrosis factor (TNF)-α have been used in combination with methotrexate in the treatment of arthritis. However, only about 50% of the patients treated with a combination of methotrexate and anti-TNF-α agents such as recombinant soluble receptors for TNF-A show clinically significant improvement. Many patients remain refractory despite treatment. Difficult treatment issues still remain for patients with rheumatoid arthritis. Many current treatments have a high incidence of side effects or cannot completely prevent disease progression. So far, no treatment is ideal, and there is no cure.

2.3. Psoriasis

Psoriasis is a chronic, inflammatory, hyperproliferative skin disease that affects approximately 1-2% of the general population with men and women affected in equal numbers. (Nevitt, G. J. et al., 1996, British J. of Dermatology 135:533-537). Approximately 150,000 new cases of psoriasis and approximately 400 deaths from psoriasis are reported each year (Stern, R. S., 1995, Dermatol. Clin. 13:717-722). The impact of psoriasis on the lives of patients goes beyond the effects on their physical appearance; it can also negatively impact their physical capacity and longevity. The most common type of psoriasis is chronic plaque syndrome. The condition is chronic for many sufferers and consists of periods of remission and relapse during the course of the disease (Ashcroft, D. M., et al., 2000, J. of Clin. Pharm. And Therap. 25: 1-10).

Psoriasis is characterized by indurated, erythematous scaling plaques most commonly located on the scalp or the extensor aspects of the elbows and knees, but may occur at any skin site.

The present treatment options currently available for psoriasis include topical agents, phototherapy and systemic agents. Topical treatments are first-line therapy for patients with mild to moderate plaque psoriasis. Systemic treatment is generally prescribed for severe cases of psoriasis where topical therapy is either impractical or ineffective. Phototherapy can be administered either alone or in combination with either topical or systemic agents. In selecting a suitable treatment, consideration should be given to the overall severity of the disease, the body areas involved, that patient's age, sex, general health, previous treatment and preferences.

Topical agents available for the treatment of psoriasis include emollients, keratolytics, coal tar, topical corticosteroids, dithranol (anthralin), topical vitamin D₃ analogues and tazarotene. Unfortunately, these topical agents are associated with side effects such as irritation, toxicity and possible carcinogenicity (Ashcroft, D. M., et al., 2000, J. of Clin. Pharm. and Therap. 25: 1-10).

Examples of phototherapy for psoriasis include ultraviolet B radiation (UVB) phototherapy and ultraviolet A photochemotherapy (PUVA). UVB phototherapy employs broadband (290-320 nm) sources and is useful in the management of moderate to severe psoriasis and is generally administered to patients whose disease is refractory to topical therapy. Treatment is usually administered two to three times a week with coal tar often being applied prior to exposure. UVB phototherapy must be carefully regulated, however, due to the short-tem risks of erythema and vesiculation and the long-term risks or premature skin aging. PUVA therapy combines long wave (320-400 nm) ultraviolet A irradiation with oral or topical administration of psoralens. The two psoralens traditionally used, 5- and 8-methoxypsoralen (MOP) are believed to intercalate into DNA and inhibit cell proliferation upon activation by UVA radiation. PUVA therapy is generally administered twice weekly. Unfortunately, PUVA commonly causes short-term risks such as nausea, erythema, headache and skin pain as well as long-term risks of actinic keratoses, premature ageing of the skin, irregular pigmentation and squamous cell carcinoma which is reported in a quarter of patients (Stern, R. S., 1994, Cancer 73:2759-2764).

Systemic agents currently used to treat psoriasis include methotrexate (MTX), cyclosporin, acitretin and hydroxyurea. There are adverse side effects associated with each of these agents, however, and most are unavailable to pregnant patients. In particular, methotrexate, which is considered to be the ‘gold standard’ for treatment of severe psoriasis, carries a risk of hepatotoxicity with long-term use. In addition, it is recommended that patients have a liner biopsy performed at or near the start of each treatment and after each cumulative dose of 1.0-1.5 mg MTX (Roenigk, H. H. et al., 1988, J. of the Am. Acad. Of Dermatology).

When patients are provided with information regarding the possible adverse effects of the currently available therapies for psoriasis, many often choose to live with the condition rather than undergo treatment (Greaves M. W., 1995, New England J. of Medicine 332:581-588).

Citation or discussion of a reference herein shall not be construed as an admission that such is prior art to the present invention.

### 3. SUMMARY OF THE INVENTION

The invention encompasses methods of administering CD2 antagonists such that efficacy is improved while safety is not compromised. The invention provides methods of treatment or prevention utilizing CD2 antagonists to achieve a desired immune response by dosing CD2 antagonists and/or monitoring lymphocyte counts. The invention encompasses methods that utilize sub-saturating levels of CD2 binding molecules in patients having autoimmune disorders or inflammatory disorders. The invention also encompasses the use of a certain specific dosage or dosages of a CD2 antagonist which is either more efficacious or safer or both. Further, the invention encompasses the administration of CD2 antagonists to achieve transient decreases in lymphocyte counts which ameliorate the symptoms of an autoimmune disorder or inflammatory disorder without inducing or while reducing the adverse side effects associated with the administration of immunologically active compounds such as proteins or antibodies.

The invention provides methods of preventing, treating or ameliorating an autoimmune disorder or an inflammatory disorder or one or more symptoms thereof, said method comprising administering to a subject in need thereof a dose of a prophylactically or therapeutically effective amount of one or more CD2 antagonists, wherein administration of said dose results in a mean absolute lymphocyte count of approximately 500 cells/μl to below 1200 cells/μl. Preferably, a subsequent dose is administered to the subject when the mean absolute lymphocyte count increases to approximately 1250 cells/μl, approximately 1300 cells/μl, approximately 1300 cells/μl, approximately 1350 cells/μl, approximately 1400 cells/μl, approximately 1450 cells/μl, approximately 1500 cells/μl, approximately 1550 cells/μl, approximately 1600 cells/μl or more.

The invention provides methods of preventing, treating or ameliorating an autoimmune disorder or an inflammatory disorder or one or more symptoms thereof, said method comprising administering to a subject in need thereof a dose of a prophylactically or therapeutically effective amount of one or more CD2 binding molecules, wherein administration of said dose results in a mean absolute lymphocyte count of approximately 500 cells/μl to below 1200 cells/μl. Preferably, a subsequent dose is administered to the subject when the mean absolute lymphocyte count increases to approximately 1250 cells/μl, approximately 1300 cells/μl, approximately 1300 cells/μl, approximately 1350 cells/μl, approximately 1400 cells/μl, approximately 1450 cells/μl, approximately 1500 cells/μl, approximately 1550 cells/μl, approximately 1600 cells/μl or more.

In a specific embodiment, the invention provides a method of preventing, treating or ameliorating an autoimmune disorder or an inflammatory disorder or one or more symptoms thereof, said method comprising administering to a subject in need thereof a dose of a prophylactically or therapeutically effective amount of a CD2 binding molecule, wherein administration of said dose results in a mean absolute lymphocyte count of approximately 500 cells/μl, preferably approximately 550 cells/μl, approximately 600 cells/μl, approximately 650 cells/μl, approximately 700 cells/μl, approximately 750 cells/μl, approximately 800 cells/μl, approximately 850 cells/μl, approximately 900 cells/μl, approximately 950 cells/μl, approximately 1000 cells/μl, approximately 1050 cells/μl, approximately 1100 cells/μl, approximately 1200 cells/μl, or approximately 1250 cells/μl. In accordance with this embodiment, the CD2 binding molecule may be a peptide, polypeptide, protein, fusion protein or antibody that immunospecifically binds to a CD2 polypeptide. Preferably, the CD2 binding molecule is an antibody, more preferably human or humanized antibody, and most preferably MEDI-507 or an antigen-binding fragment thereof.

The invention provides methods of preventing, treating or ameliorating an autoimmune disorder or an inflammatory disorder or one or more symptoms thereof, said methods comprising administering to a subject in need thereof a dose of a prophylactically or therapeutically effective amount of one or more CD2 antagonists, wherein administration of said dose results in an approximately 10% to approximately 60% reduction in said subject's mean absolute lymphocyte count relative to said subject's mean absolute lymphocyte count prior to the administration of said dose. In a specific embodiment, the invention provides a method of preventing, treating or ameliorating an autoimmune disorder or an inflammatory disorder or one or more symptoms thereof, said method comprising administering to a subject in need thereof a dose of a prophylactically or therapeutically effective amount of one or more CD2 antagonists, wherein administration of said dose results in an approximately 10%, preferably an approximately 15%, an approximately 20%, an approximately 25%, an approximately 30%, an approximately 35%, an approximately 40%, an approximately 45%, an approximately 50%, an approximately 55% or an approximately 60% reduction in said subject's mean absolute lymphocyte count relative to said subject's mean absolute lymphocyte count prior to the administration of said dose.

The invention provides methods of preventing, treating or ameliorating an autoimmune disorder or an inflammatory disorder or one or more symptoms thereof, said methods comprising administering to a subject in need thereof a dose of a prophylactically or therapeutically effective amount of one or more CD2 binding molecules, wherein administration of said dose results in an approximately 10% to approximately 60% reduction in said subject's mean absolute lymphocyte count relative to said subject's mean absolute lymphocyte count prior to the administration of said dose. In a specific embodiment, the invention provides a method of preventing, treating or ameliorating an autoimmune disorder or an inflammatory disorder or one or more symptoms thereof, said method comprising administering to a subject in need thereof a dose of a prophylactically or therapeutically effective amount of a CD2 binding molecule, wherein administration of said dose results in an approximately 10%, preferably an approximately 15%, an approximately 20%, an approximately 25%, an approximately 30%, an approximately 35%, an approximately 40%, an approximately 45%, an approximately 50%, an approximately 55% or an approximately 60% reduction in said subject's mean absolute lymphocyte count relative to said subject's mean absolute lymphocyte count prior to the administration of said dose. In accordance with this embodiment, the CD2 binding molecule may be a peptide, polypeptide, protein, fusion protein or antibody that immunospecifically binds to a CD2 polypeptide. Preferably, the CD2 binding molecule is an antibody, more preferably human or humanized antibody, and most preferably MEDI-507 or an antigen-binding fragment thereof.

The invention provides methods of preventing, treating or ameliorating an autoimmune disorder or an inflammatory disorder or one or more symptoms thereof, said methods comprising administering to a subject in need thereof a first dose of a prophylactically or therapeutically effective amount of one or more CD2 antagonists and administering to said subject one or more subsequent doses of a prophylactically or herapeutically effective amount of one or more CD2 antagonists after administration of said first dose, wherein administration of said first dose results in a mean absolute lymphocyte count of approximately 500 cells/μl to below 120 ells/ml and administration of said subsequent doses maintain a mean absolute lymphocyte count of approximately 500 cells/μl to below 1200 cells/μl. The prophylactically or therapeutically effective amount of the first dose and one or more subsequent doses of CD2 antagonists may be the same or different. Further, the route of administration of the first dose and one or more subsequent doses of CD2 antagonists may be the same or different. Preferably, the subsequent doses are administered thrice a week, twice a week, once a week, once every 2 weeks, once every 3 weeks, once every 4 weeks, once every 6 weeks, once every 8 weeks, or once every 12 weeks.

In a specific embodiment, the invention provides method of preventing, treating or ameliorating an autoimmune disorder or an inflammatory disorder or one or more symptoms thereof, said method comprising administering to a subject in need thereof a first dose of a prophylactically or therapeutically effective amount of a CD2 antagonist and administering to said subject one or more subsequent doses of a prophylactically or therapeutically effective amount of a CD2 antagonist after administration of said first dose, wherein administration of said first dose results in a mean absolute lymphocyte count of approximately 500 cells/μl, preferably approximately 550 cells/μl, approximately 600 cells/μl, approximately 650 cells/μl, approximately 700 cells/μl, approximately 750 cells/μl, approximately 800 cells/μl, approximately 850 cells/μl, approximately 900 cells/μl, approximately 950 cells/μl, approximately 1000 cells/μl, approximately 1050 cells/μl, approximately 1100 cells/μl, approximately 1200 cells/μl, approximately 1250 cells/μl and administration of said subsequent doses maintain a mean absolute lymphocyte count of approximately 500 cells/μl, preferably approximately 550 cells/μl, approximately 600 cells/μl, approximately 650 cells/μl, approximately 700 cells/μl, approximately 750 cells/μl, approximately 800 cells/μl, approximately 850 cells/μl, approximately 900 cells/μl, approximately 950 cells/μl, approximately 1000 cells/μl, approximately 1050 cells/μl, approximately 1100 cells/μl, or approximately 1200 cells/μl, or approximately 1250 cells/μl.

The invention provides methods of preventing, treating or ameliorating an autoimmune disorder or an inflammatory disorder or one or more symptoms thereof, said methods comprising administering to a subject in need thereof a first dose of a prophylactically or therapeutically effective amount of one or more CD2 binding molecules and administering to said subject one or more subsequent doses of a prophylactically or therapeutically effective amount of one or more CD2 binding molecules after administration of said first dose, wherein administration of said first dose results in a mean absolute lymphocyte count of approximately 500 cells/μl to below 1200 cells/ml and administration of said subsequent doses maintain a mean absolute lymphocyte count of approximately 500 cells/μl to below 1200 cells/μl. The prophylactically or therapeutically effective amount of the first dose and one or more subsequent doses of CD2 binding molecules may be the same or different. Further, the route of administration of the first dose and one or more subsequent doses of CD2 binding molecules may be the same or different. Preferably, said subsequent doses are administered thrice a week, twice a week, once a week, once every 2 weeks, once every 3 weeks, once every 4 weeks, once every 6 weeks, once every 8 weeks, or once every 12 weeks.

In a specific embodiment, the invention provides method of preventing, treating or ameliorating an autoimmune disorder or an inflammatory disorder or one or more symptoms thereof, said method comprising administering to a subject in need thereof a first dose of a prophylactically or therapeutically effective amount of a CD2 binding molecule and administering to said subject one or more subsequent doses of a prophylactically or therapeutically effective amount of a CD2 binding molecule after administration of said first dose, wherein administration of said first dose results in a mean absolute lymphocyte count of approximately 500 cells/μl, preferably approximately 550 cells/μl, approximately 600 cells/μl, approximately 650 cells/μl, approximately 700 cells/μl, approximately 750 cells/μl, approximately 800 cells/μl, approximately 850 cells/μl, approximately 900 cells/μl, approximately 950 cells/μl, approximately 1000 cells/μl, approximately 1050 cells/μl, approximately 1100 cells/μl, approximately 1200 cells/μl, approximately 1250 cells/μl and administration of said subsequent doses maintain a mean absolute lymphocyte count of approximately 500 cells/μl, preferably approximately 550 cells/μl, approximately 600 cells/μl, approximately 650 cells/μl, approximately 700 cells/μl, approximately 750 cells/μl, approximately 800 cells/μl, approximately 850 cells/μl, approximately 900 cells/μl, approximately 950 cells/μl, approximately 1000 cells/μl, approximately 1050 cells/μl, approximately 1100 cells/μl, or approximately 1200 cells/μl, or approximately 1250 cells/μl. In accordance with this embodiment, the CD2 binding molecule may be a peptide, polypeptide, protein, fusion protein or antibody that immunospecifically binds to a CD2 polypeptide. Preferably, the CD2 binding molecule is an antibody, more preferably human or humanized antibody, and most preferably MEDI-507 or an antigen-binding fragment hereof.

The invention provides methods of preventing, treating or ameliorating an autoimmune disorder or an inflammatory disorder or one or more symptoms thereof, said methods comprising administering to a subject in need thereof a first dose of a prophylactically or therapeutically effective amount of one or more CD2 antagonists and administering to said subject one or more subsequent doses of a prophylactically or therapeutically effective amount of one or more CD2 antagonists after administration of said first dose, wherein administration of said subsequent doses maintain a mean absolute lymphocyte count of approximately 500 cells/μl to below 1200 cells/μl. The prophylactically or therapeutically effective amount of the first dose and one or more subsequent doses of CD2 antagonists may be the same or different. Further, the route of administration of the first dose and one or more subsequent doses of CD2 antagonists may be the same or different. Preferably, said subsequent doses are administered thrice a week, twice a week, once a week, once every 2 weeks, once every 3 weeks, once every 4 weeks, once every 6 weeks, once every 8 weeks, or once every 12 weeks.

In a specific embodiment, the invention provides a method of preventing, treating or ameliorating an autoimmune disorder or an inflammatory disorder or one or more symptoms thereof, said methods comprising administering to a subject in need thereof a first dose of a prophylactically or therapeutically effective amount of a CD2 antagonist and administering to said subject one or more subsequent doses of a prophylactically or therapeutically effective amount of a CD2 antagonist after administration of said first dose, wherein administration of said subsequent doses maintain a mean absolute lymphocyte count of approximately 500 cells/μl preferably approximately 550 cells/μl, approximately 600 cells/μl, approximately 650 cells/μl, approximately 700 cells/μl, approximately 750 cells/μl, approximately 800 cells/μl, approximately 850 cells/μl, approximately 900 cells/μl, approximately 950 cells/μl, approximately 1000 cells/μl, approximately 1050 cells/μl, approximately 1100 cells/μl, or approximately 1200 cells/μl, or approximately 1250 cells/μl. In accordance with this embodiment, said subsequent doses are administered thrice a week, twice a week, once a week, once every 2 weeks, once every 3 weeks, once every 4 weeks, once every 6 weeks, once every 8 weeks, or once every 12 weeks.

The invention provides methods of preventing, treating or ameliorating an autoimmune disorder or an inflammatory disorder or one or more symptoms thereof, said methods comprising administering to a subject in need thereof a first dose of a prophylactically or therapeutically effective amount of one or more CD2 binding molecules and administering to said subject one or more subsequent doses of a prophylactically or therapeutically effective amount of one or more CD2 binding molecules after administration of said first dose, wherein administration of said subsequent doses maintain a mean absolute lymphocyte count of approximately 500 cells/μl to below 1200 cells/μl. The prophylactically or therapeutically effective amount of the first dose and one or more subsequent doses of CD2 binding molecules may be the same or different. Further, the route of administration of the first dose and one or more subsequent doses of CD2 binding molecules may be the same or different. Preferably, the subsequent doses are administered thrice a week, twice a week, once a week, once every 2 weeks, once every 3 weeks, once every 4 weeks, once every 6 weeks, once every 8 weeks, or once every 12 weeks.

In a specific embodiment, the invention provides a method of preventing, treating or ameliorating an autoimmune disorder or an inflammatory disorder or one or more symptoms thereof, said methods comprising administering to a subject in need thereof a first dose of a prophylactically or therapeutically effective amount of a CD2 binding molecule and administering to said subject one or more subsequent doses of a prophylactically or therapeutically effective amount of a CD2 binding molecule after administration of said first dose, wherein administration of said subsequent doses maintain a mean absolute lymphocyte count of approximately 500 cells/μl preferably approximately 550 cells/μl, approximately 600 cells/μl, approximately 650 cells/μl, approximately 700 cells/μl, approximately 750 cells/μl, approximately 800 cells/μl, approximately 850 cells/μl, approximately 900 cells/μl, approximately 950 cells/μl, approximately 1000 cells/μl, approximately 1050 cells/μl, approximately 1100 cells/μl, or approximately 1200 cells/μl, or approximately 1250 cells/μl. In accordance with this embodiment, the CD2 binding molecule may be a peptide, polypeptide, protein, fusion protein or antibody that immunospecifically binds to a CD2 polypeptide. Preferably, the CD2 binding molecule is an antibody, more preferably human or humanized antibody, and most preferably MEDI-507 or an antigen-binding fragment thereof.

The invention provides methods of preventing, treating or ameliorating an autoimmune disorder or an inflammatory disorder or one or more symptoms thereof, said methods comprising administering to a subject in need thereof a first dose of a prophylactically or therapeutically effective amount of one or more CD2 antagonists and administering to said subject one or more subsequent doses of a prophylactically or therapeutically effective amount of one or more CD2 antagonists after administration of said first dose, wherein administration of said subsequent doses maintain an approximately 10% to approximately 60% reduction in said subject's mean absolute lymphocyte count relative to said subject's mean absolute lymphocyte count prior to the administration of said dose. The prophylactically or therapeutically effective amount of the first dose and one or more subsequent doses of CD2 antagonists may be the same or different. Further, the route of administration of the first dose and one or more subsequent doses of CD2 antagonists may be the same or different. Preferably, the subsequent doses are administered thrice a week, twice a week, once a week, once every 2 weeks, once every 3 weeks, once every 4 weeks, once every 6 weeks, once every 8 weeks, or once every 12 weeks.

In a specific embodiment, the invention provides a method of preventing, treating or ameliorating an autoimmune disorder or an inflammatory disorder or one or more symptoms thereof, said method comprising administering to a subject in need thereof a first dose of a prophylactically or therapeutically effective amount of a CD2 antagonist and administering to said subject one or more subsequent doses of a prophylactically or therapeutically effective amount of a CD2 antagonist after administration of said first dose, wherein administration of said subsequent doses maintain an approximately 10%, preferably an approximately 15%, an approximately 20%, an approximately 25%, an approximately 30%, an approximately 35%, an approximately 40%, an approximately 45%, an approximately 50%, an approximately 55% or an approximately 60% reduction in said subject's mean absolute lymphocyte count relative to said subject's mean absolute lymphocyte count prior to the administration of said dose. In accordance with this embodiment, said subsequent doses are administered thrice a week, twice a week, once a week, once every 2 weeks, once every 3 weeks, once every 4 weeks, once every 6 weeks, once every 8 weeks, or once every 12 weeks.

The invention provides methods of preventing, treating or ameliorating an autoimmune disorder or an inflammatory disorder or one or more symptoms thereof, said methods comprising administering to a subject in need thereof a first dose of a prophylactically or therapeutically effective amount of one or more CD2 binding molecules and administering to said subject one or more subsequent doses of a prophylactically or therapeutically effective amount of one or more CD2 binding molecules after administration of said first dose, wherein administration of said subsequent doses maintain an approximately 10% to approximately 60% reduction in said subject's mean absolute lymphocyte count relative to said subject's mean absolute lymphocyte count prior to the administration of said dose. The prophylactically or therapeutically effective amount of the first dose and one or more subsequent doses of CD2 binding molecules may be the same or different. Further, the route of administration of the first dose and one or more subsequent doses of CD2 binding molecules may be the same or different. Preferably, the subsequent doses are administered thrice a week, twice a week, once a week, once every 2 weeks, once every 3 weeks, once every 4 weeks, once every 6 weeks, once every 8 weeks, or once every 12 weeks.

In a specific embodiment, the invention provides a method of preventing, treating or ameliorating an autoimmune disorder or an inflammatory disorder or one or more symptoms thereof, said method comprising administering to a subject in need thereof a first dose of a prophylactically or therapeutically effective amount of a CD2 binding molecule and administering to said subject one or more subsequent doses of a prophylactically or therapeutically effective amount of a CD2 binding molecule after administration of said first dose, wherein administration of said subsequent doses maintain an approximately 10%, preferably an approximately 15%, an approximately 20%, an approximately 25%, an approximately 30%, an approximately 35%, an approximately 40%, an approximately 45%, an approximately 50%, an approximately 55% or an approximately 60% reduction in said subject's mean absolute lymphocyte count relative to said subject's mean absolute lymphocyte count prior to the administration of said dose. In accordance with this embodiment, the CD2 binding molecule may be a peptide, polypeptide, protein, fusion protein or antibody that immunospecifically binds to a CD2 polypeptide. Preferably, the CD2 binding molecule is an antibody, more preferably human or humanized antibody, and most preferably MEDI-507 or an antigen-binding fragment thereof.

The invention provides a method of preventing, treating or ameliorating an autoimmune disorder or an inflammatory disorder or one or more symptoms thereof, said method comprising: (a) administering to a subject in need thereof one or more doses of a prophylactically or therapeutically effective amount of one or more CD2 antagonists; and (b) monitoring the mean absolute lymphocyte count in said subject after administration of a certain number of doses and prior to the administration of a subsequent dose. The mean absolute lymphocyte count in the subject may be determined after 1, 2, 3, 4, 5, 6, 7, 8, 9, 10, 11, 12, 13, 14 or more doses of the CD2 antagonists. Preferably, the administration of one or more subsequent doses of a prophylactically or therapeutically effective amount of one or more CD2 antagonists is based upon whether the mean absolute lymphocyte count is within the range of approximately 500 cells/μl to 1200 cells/μl.

In a specific embodiment, the invention provides a method of preventing, treating or ameliorating an autoimmune disorder characterized by increased infiltration of lymphocytes into dermal or epidermal tissues, said method comprising: (a) administering to a subject in need thereof one or more doses of a prophylactically or therapeutically effective amount of one or more CD2 antagonists; and (b) monitoring the mean absolute lymphocyte count in said subject after administration of a certain number of doses and prior to the administration of a subsequent dose. In another embodiment, the invention provides a method of preventing, treating or ameliorating an autoimmune disorder or inflammatory disorder characterized by increased T cell activation and/or abnormal antigen presentation, said method comprising: (a) administering to a subject in need thereof one or more doses of a prophylactically or therapeutically effective amount of one or more CD2 antagonists; and (b) monitoring the mean absolute lymphocyte count in said subject after administration of a certain number of doses and prior to the administration of a subsequent dose. In a preferred embodiment, the invention provides a method of preventing, treating or ameliorating psoriasis or one or more symptoms thereof, said method comprising: (a) administering to a subject in need thereof one or more doses of a prophylactically or therapeutically effective amount of one or more CD2 antagonists; and (b) monitoring the mean absolute lymphocyte count in said subject after administration of a certain number of doses and prior to the administration of a subsequent dose.

The invention provides a method of preventing, treating or ameliorating an autoimmune disorder or an inflammatory disorder or one or more symptoms thereof, said method comprising: (a) administering to a subject in need thereof one or more doses of a prophylactically or therapeutically effective amount of one or more CD2 binding molecules; and (b) monitoring the mean absolute lymphocyte count in said subject after administration of a certain number of doses and prior to the administration of a subsequent dose. The mean absolute lymphocyte count in the subject may be determined after 1, 2, 3, 4, 5, 6, 7, 8, 9, 10, 11,12, 13,14 or more doses of the CD2 binding molecules. Preferably, the administration of one or more subsequent doses of a prophylactically or therapeutically effective amount of one or more CD2 binding molecules is based upon whether the lymphocyte count is within the range of approximately 500 cells/μl to 1200 cells/μl.

In a specific embodiment, the invention provides a method of preventing, treating or ameliorating an autoimmune disorder characterized by increased infiltration of lymphocytes into dermal or epidermal tissues, said method comprising: (a) administering to a subject in need thereof one or more doses of a prophylactically or therapeutically effective amount of one or more CD2 binding molecules; and (b) monitoring the mean absolute lymphocyte count in said subject after administration of a certain number of doses and prior to the administration of a subsequent dose. In another embodiment, the invention provides a method of preventing, treating or ameliorating an autoimmune disorder or inflammatory disorder characterized by increased T cell activation and/or abnormal antigen presentation, said method comprising: (a) administering to a subject in need thereof one or more doses of a prophylactically or therapeutically effective amount of one or more CD2 binding molecules; and (b) monitoring the mean absolute lymphocyte count in said subject after administration of a certain number of doses and prior to the administration of a subsequent dose. In a preferred embodiment, the invention provides a method of preventing, treating or ameliorating psoriasis or one or more symptoms thereof, said method comprising: (a) administering to a subject in need thereof one or more doses of a prophylactically or therapeutically effective amount of one or more CD2 binding molecules; and (b) monitoring the mean absolute lymphocyte count in said subject after administration of a certain number of doses and prior to the administration of a subsequent dose. The invention provides methods of preventing, treating or ameliorating an autoimmune disorder or an inflammatory disorder or one or more symptoms thereof, said methods comprising: (a) administering to a subject in need thereof one or more doses of a prophylactically or therapeutically effective amount of one or more CD2 antagonists; (b) monitoring the mean absolute lymphocyte count in said subject after the administration of one or more of said doses and prior to the administration of a subsequent dose; and (c) maintaining a mean absolute lymphocyte count of approximately 500 cells/μl to below 1200 cells/μl by repeating step (a) as necessary. The prophylactically or therapeutically effective amount of the CD2 antagonists may be the same or different. Further, the method of administration of the doses of CD2 antagonists may be the same or different. In a specific embodiment, the invention provides a method of preventing, treating or ameliorating an autoimmune disorder or an inflammatory disorder or one or more symptoms thereof, said method comprising: (a) administering to a subject in need thereof one or more doses of a prophylactically or therapeutically effective amount of one or more CD2 antagonists; (b) monitoring the mean absolute lymphocyte count in said subject after the administration of one or more of said doses and prior to the administration of a subsequent dose; and (c) maintaining a mean absolute lymphocyte count of approximately 500 cells/μl, approximately 550 cells/μl, approximately 600 cells/μl, approximately 650 cells/μl, approximately 700 cells/μl, approximately 750 cells/μl, approximately 800 cells/μl, approximately 850 cells/μl, approximately 900 cells/μl, approximately 950 cells/μl, approximately 1000 cells/μl, approximately 1050 cells/μl, approximately 1100 cells/μl, approximately 1150 cells/μl, approximately 1200 cells/μl or approximately 1250 cells/μl by repeating step (a) as necessary.

The invention provides methods of preventing, treating or ameliorating an autoimmune disorder or an inflammatory disorder or one or more symptoms thereof, said methods comprising: (a) administering to a subject in need thereof one or more doses of a prophylactically or therapeutically effective amount of one or more CD2 binding molecules; (b) monitoring the mean absolute lymphocyte count in said subject after the administration of one or more of said doses and prior to the administration of a subsequent dose; and (c) maintaining a mean absolute lymphocyte count of approximately 500 cells/μl to below 1200 cells/μl by repeating step (a) as necessary. The prophylactically or therapeutically effective amount of the CD2 binding molecules may be the same or different. Further, the method of administration of the doses of CD2 binding molecules may be the same or different. In a preferred embodiment, the CD2 binding molecule is MEDI-507 or an antigen-binding fragment thereof.

In a specific embodiment, the invention provides a method of preventing, treating or ameliorating an autoimmune disorder or an inflammatory disorder or one or more symptoms thereof, said method comprising: (a) administering to a subject in need thereof one or more doses of a prophylactically or therapeutically effective amount of one or more CD2 binding molecules; (b) monitoring the mean absolute lymphocyte count in said subject after the administration of one or more of said doses and prior to the administration of a subsequent dose; and (c) maintaining a mean absolute lymphocyte count of approximately 500 cells/μl, approximately 550 cells/μl, approximately 600 cells/μl, approximately 650 cells/μl approximately 700 cells/μl, approximately 750 cells/μl, approximately 800 cells/μl, approximately 850 cells/μl, approximately 900 cells/μl, approximately 950 cells/μl, approximately 1000 cells/μl, approximately 1050 cells/μl, approximately 1100 cells/μl, approximately 1150 cells/μl, approximately 1200 cells/μl or approximately 1250 cells/μl by repeating step (a) as necessary.

The invention provides methods of preventing, treating or ameliorating an autoimmune disorder or an inflammatory disorder or one or more symptoms thereof, said methods comprising: (a) administering to a subject in need thereof one or more doses of a prophylactically or therapeutically effective amount of one or more CD2 antagonists; (b) monitoring the mean absolute lymphocyte count of said subject after the administration of one or more of said doses and prior to the administration of a subsequent dose; and (c) maintaining a mean absolute lymphocyte count in said subject which is 10% to 60% less than the mean absolute lymphocyte count in said subject prior to the administration of said doses of a prophylactically or therapeutically effective amount of one or more CD2 antagonists by repeating step (a) as necessary. The prophylactically or therapeutically effective amount of the CD2 antagonists may be the same or different. Further, the method of administration of the doses of CD2 antagonists may be the same or different.

In a specific embodiment, the invention provides a method of preventing, treating or ameliorating an autoimmune disorder or an inflammatory disorder or one or more symptoms thereof, said method comprising: (a) administering to a subject in need thereof one or more doses of a prophylactically or therapeutically effective amount of one or more CD2 antagonists; (b) monitoring the mean absolute lymphocyte count of said subject after the administration of one or more of said doses and prior to the administration of a subsequent dose; and (c) maintaining a mean absolute lymphocyte count in said subject which is 10%, preferably 15%, 20%, 25%, 30%, 35%, 40%, 45%, 50%, 55% or 60% less than the mean absolute lymphocyte count in said subject prior to the administration of said doses of a prophylactically or therapeutically effective amount of one or more CD2 antagonists by repeating step (a) as necessary.

The invention provides methods of preventing, treating or ameliorating an autoimmune disorder or an inflammatory disorder or one or more symptoms thereof, said methods comprising: (a) administering to a subject in need thereof one or more doses of a prophylactically or therapeutically effective amount of one or more CD2 binding molecules; (b) monitoring the mean absolute lymphocyte count of said subject after the administration of one or more of said doses and prior to the administration of a subsequent dose; and (c) maintaining a mean absolute lymphocyte count in said subject which is 10% to 60% less than the mean absolute lymphocyte count in said subject prior to the administration of said doses of a prophylactically or therapeutically effective amount of one or more CD2 binding molecules by repeating step (a) as necessary. The prophylactically or therapeutically effective amount of the CD2 binding molecules may be the same or different. Further, the method of administration of the doses of CD2 binding molecules may be the same or different. In a preferred embodiment, the CD2 binding molecule is MEDI-507 or an antigen-binding fragment thereof.

In a specific embodiment, the invention provides a method of preventing, treating or ameliorating an autoimmune disorder or an inflammatory disorder or one or more symptoms thereof, said method comprising: (a) administering to a subject in need thereof one or more doses of a prophylactically or therapeutically effective amount of one or more CD2 binding molecules; (b) monitoring the mean absolute lymphocyte count of said subject after the administration of one or more of said doses and prior to the administration of a subsequent dose; and (c) maintaining a mean absolute lymphocyte count in said subject which is 10%, preferably 15%, 20%, 25%, 30%, 35%, 40%, 45%, 50%, 55% or 60% less than the mean absolute lymphocyte count in said subject prior to the administration of said doses of a prophylactically or therapeutically effective amount of one or more CD2 binding molecules by repeating step (a) as necessary.

The invention provides methods of preventing, treating or ameliorating an autoimmune disorder or an inflammatory disorder or one or more symptoms thereof, said method comprising administering to a subject in need thereof a dose of a prophylactically or therapeutically effective amount of one or more CD2 binding molecules and administering to said subject one or more subsequent doses of a prophylactically or therapeutically effective amount of one or more CD2 binding molecules after administering a prior dose, wherein said CD2 binding molecules do not inhibit the interaction between LFA-3 and CD2. Preferably, the CD2 binding molecules are antibodies that immunospecifically bind to a CD2 polypeptide such as MEDI-507 or an antigen-binding fragment thereof. Moreover, preferably the autoimmune disorder is psoriasis.

The invention provides methods of preventing, treating or ameliorating an autoimmune disorder or an inflammatory disorder or one or more symptoms thereof, said methods comprising administering to a subject in need thereof a dose of a prophylactically or therapeutically effective amount of one or more CD2 binding molecules, wherein administration of said dose results in CD2 binding molecules binding to at least 25%, at least 30%, at least 35%, at least 40%, at least 45%, at least 50%, at least 55%, at least 60%, at least 65%, at least 70%, at least 75%, at least 75%, at least 80%, at least 85% or at least 90% of the CD2 polypeptides expressed by peripheral blood lymphocytes. Preferably, a subsequent dose is administered to said subject when the percentage of CD2 polypeptides bound to CD2 binding molecules drops to 20% or less, 15% or less, or 10% or less.

In a specific embodiment, the invention provides a method of preventing, treating or ameliorating an autoimmune disorder or an inflammatory disorder or one or more symptoms thereof, said method comprising administering to a subject in need thereof a dose of a prophylactically or therapeutically effective amount of a CD2 binding molecule, wherein administration of said dose results in the CD2 binding molecule binding to at least 25%, preferably at least 30%, at least 35%, at least 40%, at least 45%, at least 50%, or at least 55% of the CD2 polypeptides expressed by peripheral blood lymphocytes for at least 1 hour, at least 2 hours, at least 4 hours, at least 6 hours, at least 8 hours, at least 10 hours, at least 12 hours, at least 16 hours, at least 24 hours, at least 48 hours, at least 72 hours, or at least 1 week. In another embodiment, the invention provides a method of preventing, treating or ameliorating an autoimmune disorder or an inflammatory disorder or one or more symptoms thereof, said method comprising subcutaneously administering to a subject in need thereof a dose of a prophylactically or therapeutically effective amount of a CD2 binding molecule, wherein administration of said dose results in the CD2 binding molecule binding to at least 25%, preferably at least 30%, at least 35%, at least 40%, at least 45%, at least 50%, or at least 55% of the CD2 polypeptides expressed by peripheral blood lymphocytes. In another embodiment, the invention provides a method of preventing, treating or ameliorating an autoimmune disorder or an inflammatory disorder or one or more symptoms thereof, said method comprising intravenously administering to a subject in need thereof a dose of a prophylactically or therapeutically effective amount of a CD2 binding molecule, wherein administration of said dose results in the CD2 binding molecule binding to at least 40%, preferably at least 45%, at least 50%, at least 55%, at least 60%, at least 65%, at least 70%, at least 75%, at least 80%, at least 85% or at least 90% of the CD2 polypeptides expressed by peripheral blood lymphocytes.

The present invention provides methods of preventing, treating or ameliorating an autoimmune disorder or an inflammatory disorder or one or more symptoms thereof, said methods comprising administering to a subject in need thereof a first dose of a prophylactically or therapeutically effective amount of one or more CD2 binding molecules and administering to said subject one or more subsequent doses of a prophylactically or therapeutically effective amount of one or more CD2 binding molecules after administration of said first dose, wherein administration of said first dose results in 25% to 90% of the CD2 polypeptides expressed by peripheral blood lymphocytes being bound to CD2 binding molecules and administration of said subsequent doses restore 25% to 90% of the CD2 polypeptides expressed by peripheral blood lymphocytes being bound by CD2 binding molecules. The prophylactically or therapeutically effective amount of the CD2 binding molecules may be the same or different. Further, the method of administration of the doses of CD2 binding molecules may be the same or different. In a preferred embodiment, the CD2 binding molecule is MEDI-507 or an antigen-binding fragment thereof.

In a specific embodiment, the present invention provides a method of preventing, treating or ameliorating an autoimmune disorder or an inflammatory disorder or one or more symptoms thereof, said methods comprising administering to a subject in need thereof a first dose of a prophylactically or therapeutically effective amount of a CD2 binding molecule and administering to said subject one or more subsequent doses of a prophylactically or therapeutically effective amount of a CD2 binding molecule after administration of said first dose, wherein administration of said first dose results in at least 25%, at least 30%, at least 35%, at least 40%, at least 45%, at least 50%, at least 55%, at least 60%, at least 65%, at least 70%, at least 75%, at least 75%, at least 80%, at least 85% or at least 90% of the CD2 polypeptides expressed by peripheral blood lymphocytes being bound to a CD2 binding molecule and administration of said subsequent doses restore at least 25%, at least 30%, at least 35%, at least 40%, at least 45%, at least 50%, at least 55%, at least 60%, at least 65%, at least 70%, at least 75%, at least 75%, at least 80%, at least 85% or at least 90% of the CD2 polypeptides expressed by peripheral blood lymphocytes being bound by a CD2 binding molecule.

The invention provides methods of preventing, treating or ameliorating an autoimmune disorder or an inflammatory disorder or one or more symptoms thereof, said methods comprising: (a) administering to a subject in need thereof one or more doses of a prophylactically or therapeutically effective amount of one or more CD2 binding molecules; and (b) monitoring the percentage of CD2 polypeptides expressed by peripheral blood lymphocytes bound by CD2 binding molecules in said subject after administration of a certain number of doses and prior to the administration of a subsequent dose. The mean absolute lymphocyte count in the subject may be determined after 1, 2, 3, 4, 5, 6, 7, 8, 9, 10, 11, 12, 13, 14 or more doses of the CD2 binding molecules. Preferably, the administration of one or more subsequent doses of a prophylactically or therapeutically effective amount of one or more CD2 binding molecules is based upon whether the percentage of CD2 polypeptides bound to a CD2 binding molecule is within the range of 25% to 90%.

In a specific embodiment, the invention provides a method of preventing, treating or ameliorating an autoimmune disorder characterized by increased infiltration of lymphocytes into dermal or epidermal tissues, said method comprising: (a) administering to a subject in need thereof one or more doses of a prophylactically or therapeutically effective amount of one or more CD2 binding molecules; and (b) monitoring the percentage of CD2 polypeptides expressed by peripheral blood lymphocytes bound by CD2 binding molecules in said subject after administration of a certain number of doses and prior to the administration of a subsequent dose. In another embodiment, the invention provides a method of preventing, treating or ameliorating an autoimmune disorder or inflammatory disorder characterized by increased T cell activation and/or abnormal antigen presentation, said method comprising: (a) administering to a subject in need thereof one or more doses of a prophylactically or therapeutically effective amount of one or more CD2 binding molecules; and (b) monitoring the percentage of CD2 polypeptides expressed by peripheral blood lymphocytes bound by CD2 binding molecules in said subject after administration of a certain number of doses and prior to the administration of a subsequent dose. In a preferred embodiment, the invention provides a method of preventing, treating or ameliorating psoriasis or one or more symptoms thereof, said method comprising: (a) administering to a subject in need thereof one or more doses of a prophylactically or therapeutically effective amount of one or more CD2 binding molecules; and (b) monitoring the percentage of CD2 polypeptides expressed by peripheral blood lymphocytes bound by CD2 binding molecules in said subject after administration of a certain number of doses and prior to the administration of a subsequent dose.

The invention provides methods of preventing, treating or ameliorating an autoimmune disorder or an inflammatory disorder or one or more symptoms thereof, said method comprising: (a) administering to a subject in need thereof one or more doses of a prophylactically or therapeutically effective amount of one or more CD2 binding molecules; (b) assessing the percentage of CD2 polypeptides bound by CD2 binding molecules after administration of one or more of said doses and prior to the administration of a subsequent dose; and (c) administering to said subject one or more subsequent doses of a prophylactically or therapeutically effective amount of one or more CD2 binding molecules when the percentage of CD2 polypeptides expressed by peripheral blood lymphocytes bound by CD2 binding molecules is approximately 20% or less, approximately 15% or less, approximately 10% or less, or approximately 5% or less. The prophylactically or therapeutically effective amount of the CD2 binding molecules may be the same or different. Further, the method of administration of the doses of CD2 binding molecules may be the same or different. In a preferred embodiment, the CD2 binding molecule is MEDI-507 or an antigen-binding fragment thereof.

The invention provides methods of preventing, treating or ameliorating an autoimmune disorder or an inflammatory disorder or one or more symptoms thereof, said methods comprising: (a) administering to a subject in need thereof one or more doses of a prophylactically or therapeutically effective amount of one or more CD2 binding molecules; (b) monitoring the percentage of CD2 polypeptides bound by CD2 binding molecules after administration of one or more of said doses and prior to the administration of a subsequent dose; and (c) maintaining a 25% to 90% receptor occupancy by said CD2 binding molecules in said subject by repeating step (a) as necessary. In a specific embodiment, the invention provides a method of preventing, treating or ameliorating an autoimmune disorder or an inflammatory disorder or one or more symptoms thereof, said methods comprising: (a) administering to a subject in need thereof one or more doses of a prophylactically or therapeutically effective amount of a CD2 binding molecule; (b) monitoring the percentage of CD2 polypeptides bound by a CD2 binding molecule after administration of one or more of said doses and prior to the administration of a subsequent dose; and (c) maintaining at least a 25%, 30%, 35%,40%,45%, 50%, 55%, 60%, 65%,70%,75%, 80%, 85% or 90% receptor occupancy by a CD2 binding molecule in said subject by repeating step (a) as necessary.

The invention provides methods of preventing, treating or ameliorating psoriasis or one or more symptoms thereof, said methods comprising administering to a subject in need thereof one or more doses of a prophylactically or therapeutically effective amount of one or more CD2 binding molecules, wherein administration of said doses results in a mean absolute lymphocyte count of approximately 500 cells/μl to below 1200 cells/μl. Preferably, the administration of said doses results in at least a 10%, preferably 15%, 20%, 25%, 30%, 35%, 40%, 45%, 50%, 55%, 60%, 65%, 70%, 75% or more reduction of said subject's Psoriasis Area and Severity Index (PASI) score or a 10%, 15%, 20%, 25%, 30%, 35%, 40%, 45%, 50%, 55%, 60%, 65%, 70%, 75% or greater improvement in the subject's quality of life. In a specific embodiment, the invention provides methods of preventing, treating or ameliorating psoriasis or one or more symptoms thereof, said method comprising administering to a subject in need thereof one or more doses of a prophylactically or therapeutically effective amount of a CD2 binding molecule, wherein administration of said doses results in a mean absolute lymphocyte count of approximately 500 cells/μl, preferably approximately 550 cells/μl, approximately 600 cells/μl, approximately 650 cells/μl, approximately 700 cells/μl, approximately 750 cells/μl, approximately 800 cells/μl, approximately 850 cells/μl, approximately 900 cells/μl, approximately 1000 cells/μl, approximately 1050 cells/μl, approximately 1100 cells/μl, approximately 1150 cells/μl, approximately 1200 cells/μl or approximately 1250 cells/μl.

The invention provides methods of preventing, treating or ameliorating psoriasis or one or more symptoms thereof, said methods comprising administering to a subject in need thereof one or more doses of a prophylactically or therapeutically effective amount of one or more CD2 binding molecules, wherein administration of said doses results in at least 25%, at least 30%, at least 35%, at least 40%, at least 45%, at least 50%, at least 55%, at least 60%, at least 65%, at least 70%, at least 75% or at least 80% of CD2 polypeptides expressed by peripheral blood lymphocytes being bound by CD2 binding molecules. Preferably, the administration of said doses results in at least a 10%, preferably 15%, 20%, 25%, 30%, 35%, 40%, 45%, 50%, 55%, 60%, 65%, 70%, 75% or more reduction of said subject's Psoriasis Area and Severity Index (PASI) score or a 10%, 15%, 20%, 25%, 30%, 35%, 40%, 45%, 50%, 55%, 60%, 65%, 70%, 75% or greater improvement in the subject's quality of life.

The invention provides methods of preventing, treating or ameliorating psoriasis in a human which avoids or reduces adverse effects associated with decreasing lymphocyte counts, said methods comprising administering doses of a prophylactically or therapeutically effective amount of one or more CD2 binding molecules, said doses being effective to achieve a reduction in said human's PASI score by at least 25%, at least 15%, at least 20%, at least 25%, at least 30%, at least 35%, at least 40%, at least 45%, at least 50%, at least 55%, at least 60%, at least 65%, at least 70%, at least 75% or more reduction of said subject's Psoriasis Area and Severity Index (PASI) score, but insufficient to cause a reduction in lymphocyte count to below 500 cells/μl. Preferably, the mean absolute lymphocyte count is between 500 cells/μl and 1200 cells/μl.

The invention provides methods of preventing, treating or ameliorating psoriasis or one or more symptoms thereof, said methods comprising administering to a subject in need thereof one or more doses of a prophylactically or therapeutically effective amount of MEDI-507. In a preferred embodiment, the invention provides a method of preventing, treating or ameliorating psoriasis or one or more symptoms thereof, said method comprising administering to a subject in need thereof one or more doses of a prophylactically or therapeutically effective amount of MEDI-507, wherein administration of said doses results in a lymphocyte count of approximately 500 cells/μl, preferably approximately 550 cells/μl, approximately 600 cells/μl, approximately 650 cells/μl, approximately 700 cells/μl, approximately 750 cells/μl, approximately 800 cells/μl, approximately 850 cells/μl, approximately 900 cells/μl, approximately 1000 cells/μl, approximately 1050 cells/μl, approximately 1100 cells/μl, approximately 1150 cells/μl, approximately 1200 cells/μl or approximately 1250 cells/μl. In another preferred embodiment, the invention provides a method of preventing, treating or ameliorating psoriasis or one or more symptoms thereof, said method comprising administering to a subject in need thereof one or more doses of a prophylactically or therapeutically effective amount of MEDI-507, wherein administration of said doses results in at least 25%, at least 30%, at least 35%, at least 40%, at least 45%, at least 50%, at least 55%, at least 60%, at least 65%, at least 70%, at least 75% or at least 80% of CD2 polypeptides expressed by peripheral blood lymphocytes being bound by MEDI-507. In accordance with these embodiments, the administration of said doses results in at least a 10%, preferably 15%, 20%, 25%, 30%, 35%, 40%, 45%, 50%, 55%, 60%, 65%, 70%, 75% or more reduction of said subject's PASI score or a 10%, 15%, 20%, 25%, 30%, 35%, 40%, 45%, 50%, 55%, 60%, 65%, 70%, 75% or greater improvement in the subject's quality of life.

The invention provides pharmaceutical compositions for use in accordance with the methods of the invention, said pharmaceutical compositions comprising one or more CD2 antagonists and a pharmaceutically acceptable carrier. In a specific embodiment, the invention provides a pharmaceutical composition for use in accordance with the methods of the invention, said pharmaceutical composition comprising one or more CD2 binding molecules. In accordance with this embodiment, the CD2 binding molecule may or may not be a fusion protein that immunospecifically binds to a CD2 polypeptide. In another embodiment, the invention provides a pharmaceutical composition for use in accordance with the methods of the invention comprising one or more fusion proteins that immunospecifically bind to CD2 polypeptides. In another embodiment, the invention provides a pharmaceutical composition for use in accordance with the methods of the invention comprising one or more antibodies that immunospecifically bind to CD2 polypeptides. In a preferred embodiment, the invention provides a pharmaceutical composition for use in accordance with the methods of the invention, said pharmaceutical composition comprising MEDI-507 or an antigen-binding fragment thereof.

The compositions and methods described herein are useful for the prevention, treatment or amelioration of autoimmune disorders including, but not limited to, alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Ménière's disease, mixed connective tissue disease, multiple sclerosis, type 1 or immune-mediated diabetes mellitus, myasthenia gravis, pemphigus vulgaris, pernicious anemia, polyarteritis nodosa, polychrondritis, polyglandular syndromes, polymyalgia rheumatica, polymyositis and dermatomyositis, primary agammaglobulinemia, primary biliary cirrhosis, psoriasis, psoriatic arthritis, Raynauld's phenomenon, Reiter's syndrome, Rheumatoid arthritis, sarcoidosis, scleroderma, Sjögren's syndrome, stiff-man syndrome, systemic lupus erythematosus, lupus erythematosus, takayasu arteritis, temporal arteristis/giant cell arteritis, ulcerative colitis, uveitis, vasculitides such as dermatitis herpetiformis vasculitis, vitiligo, and Wegener's granulomatosis. The compositions and methods described herein are particularly useful for the prevention, treatment or amelioration of autoimmune disorders characterized by increased T cell infiltration of lymphocytes into affected dermal or epidermal tissues, or autoimmune disorders characterized by increased T cell activation and/or abnormal antigen presentation.

The compositions and methods described herein are useful for the prevention, treatment or amelioration of inflammatory disorders include, but are not limited to, asthma, encephilitis, inflammatory bowel disease, chronic obstructive pulmonary disease (COPD), inflammatory osteolysis, allergic disorders, septic shock, pulmonary fibrosis, undifferentitated spondyloarthropathy, undifferentiated arthropathy, arthritis, inflammatory osteolysis, and chronic inflammation resulting from chronic viral or bacteria infections. In particular, the composition and methods described herein are useful for the prevention, treatment or amelioration of inflammatory disorders characterized by increased T cell activation and/or abnormal antigen presentation. The compositions of the invention described herein can also be applied to skin conditions characterized by increased T cell activation and/or abnormal T cell activation such as, e.g., psoriasis, ultraviolet damage, atopic dermatitis, cutaneous T cell lymphoma, allergic and irritant contact dermatitis, lichen planus, alopecia areata, pyoderma gangrenosum, vitiligo, ocular, cicatricial pemphigoid, lupus erythematous, scleroderma, and urticaria.

The present invention provides article of manufactures comprising packaging material and a pharmaceutical composition of the invention in suitable form for administration to a subject contained within said packaging material. In particular, the present invention provides article of manufactures comprising packaging material and a pharmaceutical composition of the invention in suitable form for administration to a subject contained within said packaging material wherein said pharmaceutical composition comprises one or more CD2 binding molecules, one or more prophylactic or therapeutic agents other than CD2 binding molecules, and a pharmaceutically acceptable carrier. The articles of manufacture of the invention may include instructions regarding the use or administration of a pharmaceutical composition, or other informational material that advises the physician, technician or patient on how to appropriately prevent or treat the disease or disorder in question.

In a specific embodiment, an article of manufacture comprises packaging material and a pharmaceutical agent contained within said packaging material, wherein said pharmaceutical agent comprises a CD2 binding molecule and a pharmaceutically acceptable carrier, wherein said article of manufacture includes instruction means indicating a dosing regimen comprising administering an initial dosing, and optionally administering a subsequent dose or doses, of said pharmaceutical agent to a subject suffering from one or more symptoms associated with an autoimmune disorder or an inflammatory disorder, wherein the instruction means suggests a dosing regimen comprising an initial dosing that results in CD2 binding molecules binding to at least 30% of the CD2 polypeptides expressed by the subject's peripheral blood lymphocytes for at least 1 hour after the administration of said initial dosing, and wherein the instruction means suggests a dosing interval for said dosing regimen such that any dose/doses administered subsequent to said initial dosing, if administered, is/are only administered when 20% or less of the CD2 polypeptides expressed by peripheral blood lymphocytes are bound by previously administered CD2 binding molecules. In another embodiment, an article of manufacture comprises packaging material and a pharmaceutical composition in suitable form for administration to a human contained within said packaging material, wherein said pharmaceutical composition comprises MEDI-507 or an antigen-binding fragment thereof, and a pharmaceutically acceptable carrier.

3.1. Terminology

As used herein, the term “analog” in the context of polypeptides refers to a polypeptide that possesses a similar or identical function as a second polypeptide but does not necessarily comprise a similar or identical amino acid sequence of the second polypeptide, or possess a similar or identical structure of the second polypeptide. A polypeptide that has a similar amino acid sequence refers to a second polypeptide that satisfies at least one of the following: (a) a polypeptide having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the amino acid sequence of a second polypeptide; (b) a polypeptide encoded by a nucleotide sequence that hybridizes under stringent conditions to a nucleotide sequence encoding a second polypeptide of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, or at least 150 contiguous amino acid residues; and (c) a polypeptide encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the nucleotide sequence encoding a second polypeptide. A polypeptide with similar structure to a second polypeptide refers to a polypeptide that has a similar secondary, tertiary or quaternary structure to the second polypeptide. The structure of a polypeptide can be determined by methods known to those skilled in the art, including but not limited to, peptide sequencing, X-ray crystallography, nuclear magnetic resonance, circular dichroism, and crystallographic electron microscopy.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present invention. BLAST protein searches can be performed with the XBLAST program parameters set, e.g., to score-50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

As used herein, the term “analog” in the context of a non-proteinaceous analog refers to a second organic or inorganic molecule which possess a similar or identical function as a first organic or inorganic molecule and is structurally similar to the first organic or inorganic molecule.

As used herein, the terms “antagonist” and “antagonists” refer to any protein, polypeptide, peptide, antibody, antibody fragment, large molecule, or small molecule (less than 10 kD) that blocks, inhibits, reduces or neutralizes the function, activity and/or expression of another molecule. In various embodiments, an antagonist reduces the function, activity and/or expression of another molecule by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least buffered saline (PBS).

As used herein, the terms “antibody” and “antibodies” refer to monoclonal antibodies, multispecific antibodies, human antibodies, humanized antibodies, chimeric antibodies, single-chain Fvs (scFv), single chain antibodies, Fab fragments, F(ab′) fragments, disulfide-linked Fvs (sdFv), and anti-idiotypic (anti-Id) antibodies (including, e.g., anti-Id antibodies to antibodies of the invention), and epitope-binding fragments of any of the above. In particular, antibodies include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA and IgY), class (e.g., IgG₁, IgG₂, IgG₃, IgG₄, IgA₁, and IgA₂) or subclass.

As used herein, the term “CD2 antagonist” and analogous terms refer to any protein, polypeptide, peptide, fusion protein, antibody, antibody fragment, nucleic acid molecule (e.g., a CD2 antisense nucleic acid molecule or a triple helix), organic molecule, inorganic molecule, small organic molecule, drug, or small inorganic molecule that blocks, inhibits, reduces or neutralizes a function, an activity and/or the expression of a CD2 polypeptide. In various embodiments, a CD2 antagonist reduces the function, activity and/or expression of a CD2 polypeptide by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% relative to a control such as PBS. In certain embodiments, a CD2 antagonist is not a small organic molecule. In other embodiments, a CD2 antagonist is not an antisense nucleic acid molecule or triple helix. In a preferred embodiment, a CD2 antagonist is a CD2 binding molecule. In other embodiments, a CD2 antagonist is not a CD2 binding molecule.

As used herein, the term “CD2 polypeptide” refers to a CD2 glycoprotein (a.k.a. Ti 1 or LFA-2) or fragment thereof. In a preferred embodiment, a CD2 polypeptide is the cell surface 50-55 kDa glycoprotein expressed by immune cells such as T-cells and natural killer (“NK”). The CD2 polypeptide may be from any species. The nucleotide and/or amino acid sequences of CD2 polypeptides can be found in the literature or public databases, or the nucleotide and/or amino acid sequences can be determined using cloning and sequencing techniques known to one of skill in the art. For example, the nucleotide sequence of human CD2 can be found in the GenBank database (see, e.g., Accession Nos. X06143, AH002740, and M16445).

As used herein, the term “derivative” in the context of polypeptides refers to a polypeptide that comprises an amino acid sequence which has been altered by the introduction of amino acid residue substitutions, deletions or additions. The term “derivative” as used herein also refers to a polypeptide which has been modified, i.e, by the covalent attachment of any type of molecule to the polypeptide. For example, but not by way of limitation, an antibody may be modified, e.g., by glycosylation, acetylation, pegylation, phosphorylation, amidation, derivatization by known protecting/blocking groups, proteolytic cleavage, linkage to a cellular ligand or other protein, etc. A derivative polypeptide may be produced by chemical modifications using techniques known to those of skill in the art, including, but not limited to specific chemical cleavage, acetylation, formylation, metabolic synthesis of tunicamycin, etc. Further, a derivative polypeptide may contain one or more non-classical amino acids. A polypeptide derivative possesses a similar or identical function as the polypeptide from which it was derived.

As used herein, the term “derivative” in the context of a non-proteinaceous derivative refers to a second organic or inorganic molecule that is formed based upon the structure of a first organic or inorganic molecule. A derivative of an organic molecule includes, but is not limited to, a molecule modified, e.g., by the addition or deletion of a hydroxyl, methyl, ethyl, carboxyl or amine group. An organic molecule may also be esterified, alkylated and/or phosphorylated.

As used herein, the terms “disorder” and “disease” are used interchangeably to refer to a condition in a subject. In particular, the term “autoimmune disease” is used interchangeably with the term “autoimmune disorder” to refer to a condition in a subject characterized by cellular, tissue and/or organ injury caused by an immunologic reaction of the subject to its own cells, tissues and/or organs. The term “inflammatory disease” is used interchangeably with the term “inflammatory disorder” to refer to a condition in a subject characterized by inflammation, preferably chronic inflammation. Autoimmune disorders may or may not be associated with inflammation. Moreover, inflammation may or may not be caused by an autoimmune disorder. Thus, certain disorders may be characterized as both autoimmune and inflammatory disorders.

As used herein, the term “epitopes” refers to fragments of a polypeptide or protein having antigenic or immunogenic activity in an animal, preferably in a mammal, and most preferably in a human. An epitope having immunogenic activity is a fragment of a polypeptide or protein that elicits an antibody response in an animal. An epitope having antigenic activity is a fragment of a polypeptide or protein to which an antibody immunospecifically binds as determined by any method well-known to one of skill in the art, for example by immunoassays. Antigenic epitopes need not necessarily be immunogenic.

As used herein, the term “fragment” refers to a peptide or polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, or at least contiguous 250 amino acid residues of the amino acid sequence of another polypeptide. In a specific embodiment, a fragment of a polypeptide retains at least one function of the polypeptide.

As used herein, the term “functional fragment” refers to a peptide or polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, or at least contiguous 250 amino acid residues of the amino acid sequence of second, different polypeptide, wherein said peptide or polypeptide retains at least one function of the second, different polypeptide.

As used herein, the term “fusion protein” refers to a polypeptide that comprises an amino acid sequence of a first protein or functional fragment, analog or derivative thereof, and an amino acid sequence of a heterologous protein (i.e., a second protein or functional fragment, analog or derivative thereof different than the first protein or functional fragment, analog or derivative thereof). In particular embodiments, a fusion protein comprises a CD2 binding molecule and a heterologous protein, polypeptide, or peptide.

As used herein, the term “host cell” refers to the particular subject cell transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

As used herein, the term “hybridizes under stringent conditions” describes conditions for hybridization and washing under which nucleotide sequences at least 60% (65%, 70%, preferably 75%) identical to each other typically remain hybridized to each other. Such stringent conditions are known to those skilled in the art and can be found in Current Protocols in Molecular Biology, John Wiley & Sons, N.Y. (1989), 6.3.1-6.3.6. In one, non-limiting example stringent hybridization conditions are hybridization at 6× sodium chloride/sodium citrate (SSC) at about 45° C., followed by one or more washes in 0.1× SSC, 0.2% SDS at about 68° C. In a preferred, non-limiting example stringent hybridization conditions are hybridization in 6× XSSC at about 45° C., followed by one or more washes in 0.2× SSC, 0.1% SDS at 50-65° C. (i.e., one or more washes at 50° C., 55° C., 60° C. or 65° C.). It is understood that the nucleic acids of the invention do not include nucleic acid molecules that hybridize under these conditions solely to a nucleotide sequence consisting of only A or T nucleotides.

As used herein, the term “immunospecifically binds to an antigen” and analogous terms refer to peptides, polypeptides, fusion proteins and antibodies or fragments thereof that specifically bind to an antigen or a fragment and do not specifically bind to other antigens. A peptide or polypeptide that immunospecifically binds to an antigen may bind to other peptides or polypeptides with lower affinity as determined by, e.g., immunoassays, BIAcore, or other assays known in the art. Antibodies or fragments that immunospecifically bind to an antigen may cross-reactive with related antigens. Preferably, antibodies or fragments that immunospecifically bind to an antigen do not cross-react with other antigens.

As used herein, the term “immunospecifically binds to a CD2 polypeptide” and analogous terms refer to peptides, polypeptides, fusion proteins and antibodies or fragments thereof that specifically bind to a CD2 polypeptide or a fragment thereof and do not specifically bind to other polypeptides. A peptide or polypeptide that immunospecifically binds to a CD2 polypeptide may bind to other peptides or polypeptides with lower affinity as determined by, e.g., immunoassays, BIAcore, or other assays known in the art. Antibodies or fragments that immunospecifically bind to a CD2 polypeptide may be cross-reactive with related antigens. Preferably, antibodies or fragments that immunospecifically bind to a CD2 polypeptide or fragment thereof do not cross-react with other antigens. Antibodies or fragments that immunospecifically bind to a CD2 polypeptide can be identified, for example, by immunoassays, BIAcore, or other techniques known to those of skill in the art. An antibody or fragment thereof binds specifically to a CD2 polypeptide when it binds to a CD2 polypeptide with higher affinity than to any cross-reactive antigen as determined using experimental techniques, such as radioimmunoassays (RIA) and enzyme-linked immunosorbent assays (ELISAs). See, e.g., Paul, ed., 1989, Fundamental Immunology Second Edition, Raven Press, New York at pages 332-336 for a discussion regarding antibody specificity.

As used herein, the term “isolated” in the context of a peptide, polypeptide, fusion protein or antibody refers to a peptide, polypeptide, fusion protein or antibody which is substantially free of cellular material or contaminating proteins from the cell or tissue source from which it is derived, or substantially free of chemical precursors or other chemicals when chemically synthesized. The language “substantially free of cellular material” includes preparations of a peptide, polypeptide, fusion protein or antibody in which the peptide, polypeptide, fusion protein or antibody is separated from cellular components of the cells from which it is isolated or recombinantly produced. Thus, a peptide, polypeptide, fusion protein or antibody that is substantially free of cellular material includes preparations of a peptide, polypeptide, fusion protein or antibody having less than about 30%, 20%, 10%, or 5% (by dry weight) of heterologous protein (also referred to herein as a “contaminating protein”). When the peptide, polypeptide, fusion protein or antibody is recombinantly produced, it is also preferably substantially free of culture medium, i.e., culture medium represents less than about 20%, 10%, or 5% of the volume of the protein preparation. When the peptide, polypeptide, fusion protein or antibody is produced by chemical synthesis, it is preferably substantially free of chemical precursors or other chemicals, i.e., it is separated from chemical precursors or other chemicals which are involved in the synthesis of the peptide, polypeptide, fusion protein or antibody. Accordingly such preparations of a peptide, polypeptide, fusion protein or antibody have less than about 30%, 20%, 10%, 5% (by dry weight) of chemical precursors or compounds other than the peptide, polypeptide, fusion protein or antibody of interest. In a preferred embodiment, a CD2 antagonist is isolated. In another preferred embodiment, a CD2 binding molecule is isolated.

As used herein, the term “isolated” in the context of nucleic acid molecules refers to a nucleic acid molecule which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a preferred embodiment, a nucleic acid molecule encoding a CD2 antagonist is isolated. In another preferred embodiment, a nucleic acid molecule encoding a CD2 binding molecule is isolated.

As used herein, the terms “non-responsive” and refractory” describe patients treated with a currently available prophylactic or therapeutic agent for an inflammatory disorder or an autoimmune disorder (e.g., methotrexate alone or an anti-TNF-α agent) which is not clinically adequate to relieve one or more symptoms associated with the inflammatory or autoimmune disorder. Typically, such patients suffer from severe, persistently active disease and require additional therapy to ameliorate the symptoms associated with their inflammatory or autoimmune disorder.

As used herein, the terms “nucleic acids” and “nucleotide sequences” include DNA molecules (e.g., cDNA or genomic DNA), RNA molecules (e.g., mRNA), combinations of DNA and RNA molecules or hybrid DNA/RNA molecules, and analogs of DNA or RNA molecules. Such analogs can be generated using, for example, nucleotide analogs, which include, but are not limited to, inosine or tritylated bases. Such analogs can also comprise DNA or RNA molecules comprising modified backbones that lend beneficial attributes to the molecules such as, for example, nuclease resistance or an increased ability to cross cellular membranes. The nucleic acids or nucleotide sequences can be single-stranded, double-stranded, may contain both single-stranded and double-stranded portions, and may contain triple-stranded portions, but preferably is double-stranded DNA.

As used herein, the terms “prophylactic agent” and “prophylactic agents” refer to CD2 antagonists which can be used in the prevention, treatment, management or amelioration of one or more symptoms of an autoimmune or inflammatory disease. In certain embodiments, the term “prophylactic agent” refers to CD2 binding molecules (e.g., MEDI-507).

As used herein, the term “prophylactically effective amount” refers to that amount of a CD2 antagonist sufficient to prevent the development, recurrence or onset of one or more symptoms of a disorder. In certain embodiments, the term “prophylactically effective amount” refers to the amount of a CD2 binding molecule sufficient to prevent the development, recurrence or onset of one or more symptoms of a disorder.

As used herein, the terms “prevent”, “preventing” and prevention refer to the prevention of the recurrence or onset of one or more symptoms of an autoimmune or inflammatory disorder in a subject resulting from the administration of a prophylactic or therapeutic agent.

A used herein, a “protocol” includes dosing schedules and dosing regimens. The protocols herein are methods of use and include prophylactic and therapeutic protocols.

As used herein, the phrase “side effects” encompasses unwanted and adverse effects of a prophylactic or therapeutic agent. Adverse effects are always unwanted, but unwanted effects are not necessarily adverse.

As used herein, the term “small molecules” include, but are not limited to, peptides, peptidomimetics, amino acids, amino acid analogs, polynucleotides, polynucleotide analogs, nucleotides, nucleotide analogs, organic or inorganic compounds (i.e,. including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

As used herein, the terms “subject” and “patient” are used interchangeably. As used herein, the terms “subject” and “subjects” refer to an animal, preferably a mammal including a non-primate (e.g., a cow, pig, horse, cat, dog, rat, and mouse) and a non-primate (e.g., a monkey such as a cynomolgous monkey and a human), and more preferably a human. In one embodiment, the subject is not an immunocompromised or immunosuppressed mammal, preferably a human (e.g., an HIV patient). In another embodiment, the subject is not a mammal, preferably a human, with a lymphocyte count under approximately 500 cells/μl. In another embodiment, the subject is a human that has psoriasis that is refractory to topical or steroid treatment. In another embodiment, the subject is a mammal, preferably a human, that has not been treated with an immunomodulatory agent, preferably an immunosuppressant agent, to prevent, treat or ameliorate one or more symptoms of psoriasis. In an alternative embodiment, the subject is a mammal, preferably a human, who has been treated or who is being treated with another immunomodulatory agent to prevent, treat or ameliorate one or more symptoms of psoriasis. In a preferred embodiment, the subject is a human subject.

As used herein, the terms “therapeutic agent” and “therapeutic agents” refer to CD2 antagonists which can be used in the prevention, treatment, management or amelioration of one or more symptoms of an autoimmune or inflammatory disease. In certain embodiments, the term “therapeutic agent” refers to CD2 binding molecules (e.g., MEDI-7).

As used herein, the term “therapeutically effective amount” refers to that amount of a therapeutic agent sufficient to result in amelioration of one or more symptoms of a disorder. With respect to the treatment of psoriasis, a therapeutically effective amount preferably refers to the amount of a therapeutic agent that reduces a human's Psoriasis Area and Severity Index (PASI) score by at least 20%, at least 35%, at least 30%, at least 40%, at least 45%, at least 50%, at least 55%, at least 60%, at least 65%, at least 70%, at least 75%, at least 80%, or at least 85%. Alternatively, with respect to the treatment of psoriasis, a therapeutically effective amount preferably refers to the amount of a therapeutic agent that improves a human's global assessment score by at least 25%, at least 35%, at least 30%, at least 40%, at least 45%, at least 50%, at least 55%, at least 60%, at least 65%, at least 70%, at least 75%, at least 80%, at least 85%, at least 90%, or at least 95%.

As used herein, the terms “treat”, “treatment” and “treating” refer to the amelioration of one or more symptoms associated with an autoimmune or inflammatory disorder that results from the administration of one or more CD2 antagonists. In particular, such terms refer to the amelioration of one or more symptoms associated with an autoimmune or inflammatory disorder that results from the administration of one or more CD2 binding molecules. In certain embodiments, such terms refer to a reduction in the swelling of one or more joints, or a reduction in the pain associated with an autoimmune or inflammatory disorder resulting from the administration of one or more CD2 antagonists, preferably one or more CD2 binding molecules, to a subject with such a disorder. In other embodiments, such terms refer to a reduction in a human's PASI score. In other embodiments, such terms refer to an improvement in a human's global assessment score.

### 4. DETAILED DESCRIPTION OF THE INVENTION

The invention encompasses methods of administering a CD2 antagonist to a subject with an autoimmune or inflammatory disorder such that the efficacy of said CD2 antagonist is improved while the safety of said subject is not compromised. The invention provides methods of achieving a desired immune response in a subject with an autoimmune or inflammatory disorder, without inducing or reducing the adverse side effects associated with the administration of an immunomodulatory agent. Examples of a desired immune response include, but are not limited to, a transient decrease in lymphocyte counts (preferably T cell counts), a transient decrease in antibody production, a transient decrease in cytokine production, or a modification in the cytokine profile in a subject with an autoimmune disorder or an inflammatory disorder. The invention also provides methods of determining whether or not a subject with an autoimmune or inflammatory disorder requires the administration of a specific dosage and/or additional dosages of a CD2 antagonist, said methods comprising assessing the percentage of CD2 polypeptides bound to a CD2 binding molecule and/or assessing the mean absolute lymphocyte count, preferably T cell count, in said subject. Accordingly, the present invention provides methods of preventing, treating or ameliorating an autoimmune or inflammatory disorder or one or more symptoms thereof, said methods comprising administering to a subject in need thereof one or more specific dosages to achieve a particular mean absolute lymphocyte count and/or a particular percentage of receptor occupancy by CD2 antagonists.

The invention provides methods of preventing, treating or ameliorating an autoimmune disorder or an inflammatory disorder or one or more symptoms thereof, said method comprising administering to a subject in need thereof a dose of a prophylactically or therapeutically effective amount of one or more CD2 antagonists, wherein administration of said dose results in a mean absolute lymphocyte count of approximately 500 cells/μl to below 1200 cells/μl. In particular, the invention provides methods of preventing, treating or ameliorating an autoimmune disorder or an inflammatory disorder or one or more symptoms thereof, said method comprising administering to a subject in need thereof a dose of a prophylactically or therapeutically effective amount of a CD2 binding molecule, wherein administration of said dose results in a mean absolute lymphocyte count of approximately 500 cells/μl to below 1200 cells/μl. Preferably, a subsequent dose is administered to the subject when the mean absolute lymphocyte count increases to approximately 1250 cells/μl, approximately 1300 cells/μl, approximately 1300 cells/μl, approximately 1350 cells/μl, approximately 1400 cells/μl, approximately 1450 cells/μl, approximately 1500 cells/μl, approximately 1550 cells/μl, approximately 1600 cells/μl or more.

The invention provides methods of preventing, treating or ameliorating an autoimmune disorder or an inflammatory disorder or one or more symptoms thereof, said methods comprising administering to a subject in need thereof a dose of a prophylactically or therapeutically effective amount of one or more CD2 antagonists, wherein administration of said dose results in an approximately 10% to approximately 60% reduction in said subject's mean absolute lymphocyte count relative to said subject's mean absolute lymphocyte count prior to the administration of said dose. More particularly, the invention provides methods of preventing, treating or ameliorating an autoimmune disorder or an inflammatory disorder or one or more symptoms thereof, said methods comprising administering to a subject in need thereof a dose of a prophylactically or therapeutically effective amount of a CD2 binding molecules, wherein administration of said dose results in an approximately 10% to approximately 60% reduction in said subject's mean absolute lymphocyte count relative to said subject's mean absolute lymphocyte count prior to the administration of said dose. Preferably, the CD2 binding molecule is an antibody, more preferably human or humanized antibody, and most preferably MEDI-507 or an antigen-binding fragment thereof.

The invention provides methods of preventing, treating or ameliorating an autoimmune disorder or an inflammatory disorder or one or more symptoms thereof, said methods comprising administering to a subject in need thereof a first dose of a prophylactically or therapeutically effective amount of one or more CD2 antagonists and administering to said subject one or more subsequent doses of a prophylactically or therapeutically effective amount of one or more CD2 antagonists after administration of said first dose, wherein administration of said first dose results in a mean absolute lymphocyte count of approximately 500 cells/μl to below 1200 cells/ml and administration of said subsequent doses maintain a mean absolute lymphocyte count of approximately 500 cells/μl to below 1200 cells/μl. In particular, the invention provides methods of preventing, treating or ameliorating an autoimmune disorder or an inflammatory disorder or one or more symptoms thereof, said methods comprising administering to a subject in need thereof a first dose of a prophylactically or therapeutically effective amount of a CD2 binding molecule and administering to said subject one or more subsequent doses of a prophylactically or therapeutically effective amount of a CD2 binding molecule after administration of said first dose, wherein administration of said first dose results in a mean absolute lymphocyte count of approximately 500 cells/μl to below 1200 cells/ml and administration of said subsequent doses maintain a mean absolute lymphocyte count of approximately 500 cells/μl to below 1200 cells/μl. Preferably, the CD2 binding molecule is an antibody, more preferably human or humanized antibody, and most preferably MEDI-507 or an antigen-binding fragment thereof.

The invention provides methods of preventing, treating or ameliorating an autoimmune disorder or an inflammatory disorder or one or more symptoms thereof, said methods comprising administering to a subject in need thereof a first dose of a prophylactically or therapeutically effective amount of one or more CD2 antagonists and administering to said subject one or more subsequent doses of a prophylactically or therapeutically effective amount of one or more CD2 antagonists after administration of said first dose, wherein administration of said subsequent doses maintain a mean absolute lymphocyte count of approximately 500 cells/μl to below 1200 cells/μl. More particularly, the invention provides methods of preventing, treating or ameliorating an autoimmune disorder or an inflammatory disorder or one or more symptoms thereof, said methods comprising administering to a subject in need thereof a first dose of a prophylactically or therapeutically effective amount of a CD2 binding molecule and administering to said subject one or more subsequent doses of a prophylactically or therapeutically effective amount of a CD2 binding molecule after administration of said first dose, wherein administration of said subsequent doses maintain a mean absolute lymphocyte count of approximately 500 cells/μl to below 1200 cells/μl. Preferably, the CD2 binding molecule is an antibody, more preferably human or humanized antibody, and most preferably MEDI-507 or an antigen-binding fragment thereof.

The invention provides methods of preventing, treating or ameliorating an autoimmune disorder or an inflammatory disorder or one or more symptoms thereof, said methods comprising administering to a subject in need thereof a first dose of a prophylactically or therapeutically effective amount of one or more CD2 antagonists and administering to said subject one or more subsequent doses of a prophylactically or therapeutically effective amount of one or more CD2 antagonists after administration of said first dose, wherein administration of said subsequent doses maintain an approximately 10% to approximately 60% reduction in said subject's mean absolute lymphocyte count relative to said subject's mean absolute lymphocyte count prior to the administration of said dose. In particular, the invention provides methods of preventing, treating or ameliorating an autoimmune disorder or an inflammatory disorder or one or more symptoms thereof, said methods comprising administering to a subject in need thereof a first dose of a prophylactically or therapeutically effective amount of a CD2 binding molecule and administering to said subject one or more subsequent doses of a prophylactically or therapeutically effective amount of a CD2 binding molecule after administration of said first dose, wherein administration of said subsequent doses maintain an approximately 10% to approximately 60% reduction in said subject's mean absolute lymphocyte count relative to said subject's mean absolute lymphocyte count prior to the administration of said dose.

The invention provides a method of preventing, treating or ameliorating an autoimmune disorder or an inflammatory disorder or one or more symptoms thereof, said method comprising: (a) administering to a subject in need thereof one or more doses of a prophylactically or therapeutically effective amount of one or more CD2 antagonists; and (b) monitoring the mean absolute lymphocyte count in said subject after administration of a certain number of doses and prior to the administration of a subsequent dose. The mean absolute lymphocyte count in the subject may be determined after 1, 2, 3, 4, 5, 6, 7, 8, 9, 10, 11, 12, 13, 14 or more doses of the CD2 antagonists. Preferably, the administration of one or more subsequent doses of a prophylactically or therapeutically effective amount of one or more CD2 antagonists is based upon whether the mean absolute lymphocyte count is within the range of approximately 500 cells/μl to 1200 cells/μl.

The invention provides a method of preventing, treating or ameliorating an autoimmune disorder or an inflammatory disorder or one or more symptoms thereof, said method comprising: (a) administering to a subject in need thereof one or more doses of a prophylactically or therapeutically effective amount of one or more CD2 binding molecules; and (b) monitoring the mean absolute lymphocyte count in said subject after administration of a certain number of doses and prior to the administration of a subsequent dose. The mean absolute lymphocyte count in the subject may be determined after 1, 2, 3, 4, 5, 6, 7, 8, 9, 10, 11, 12, 13, 14 or more doses of the CD2 binding molecules. Preferably, the administration of one or more subsequent doses of a prophylactically or therapeutically effective amount of one or more CD2 binding molecules is based upon whether the lymphocyte count is within the range of approximately 500 cells/μl to 1200 cells/μl.

The invention provides methods of preventing, treating or ameliorating an autoimmune disorder or an inflammatory disorder or one or more symptoms thereof, said methods comprising: (a) administering to a subject in need thereof one or more doses of a prophylactically or therapeutically effective amount of one or more CD2 antagonists; (b) monitoring the mean absolute lymphocyte count in said subject after the administration of one or more of said doses and prior to the administration of a subsequent dose; and (c) maintaining a mean absolute lymphocyte count of approximately 500 cells/μl to below 1200 cells/μl by repeating step (a) as necessary. In particular, the invention provides methods of preventing, treating or ameliorating an autoimmune disorder or an inflammatory disorder or one or more symptoms thereof, said methods comprising: (a) administering to a subject in need thereof one or more doses of a prophylactically or therapeutically effective amount of a CD2 binding molecule; (b) monitoring the mean absolute lymphocyte count in said subject after the administration of one or more of said doses and prior to the administration of a subsequent dose; and (c) maintaining a mean absolute lymphocyte count of approximately 500 cells/μl to below 1200 cells/μl by repeating step (a) as necessary. In a preferred embodiment, the CD2 binding molecule is MEDI-507 or an antigen-binding fragment thereof.

The invention provides methods of preventing, treating or ameliorating an autoimmune disorder or an inflammatory disorder or one or more symptoms thereof, said methods comprising: (a) administering to a subject in need thereof one or more doses of a prophylactically or therapeutically effective amount of one or more CD2 antagonists; (b) monitoring the mean absolute lymphocyte count of said subject after the administration of one or more of said doses and prior to the administration of a subsequent dose; and (c) maintaining a mean absolute lymphocyte count in said subject which is 10% to 60% less than the mean absolute lymphocyte count in said subject prior to the administration of said doses of a prophylactically or therapeutically effective amount of one or more CD2 antagonists by repeating step (a) as necessary. More particularly, the invention provides methods of preventing, treating or ameliorating an autoimmune disorder or an inflammatory disorder or one or more symptoms thereof, said methods comprising: (a) administering to a subject in need thereof one or more doses of a prophylactically or therapeutically effective amount of a CD2 binding molecule; (b) monitoring the mean absolute lymphocyte count of said subject after the administration of one or more of said doses and prior to the administration of a subsequent dose; and (c) maintaining a mean absolute lymphocyte count in said subject which is 10% to 60% less than the mean absolute lymphocyte count in said subject prior to the administration of said doses of a prophylactically or therapeutically effective amount of the CD2 binding molecule by repeating step (a) as necessary. In a preferred embodiment, the CD2 binding molecule is MEDI-507 or an antigen-binding fragment thereof.

The invention provides methods of preventing, treating or ameliorating an autoimmune disorder or an inflammatory disorder or one or more symptoms thereof, said method comprising administering to a subject in need thereof a dose of a prophylactically or therapeutically effective amount of one or more CD2 binding molecules and administering to said subject one or more subsequent doses of a prophylactically or therapeutically effective amount of one or more CD2 binding molecules after administering a prior dose, wherein said CD2 binding molecules do not inhibit the interaction between LFA-3 and CD2. Preferably, the CD2 binding molecules are antibodies that immunospecifically bind to a CD2 polypeptide such as MEDI-507 or an antigen-binding fragment thereof. Moreover, preferably the autoimmune disorder is psoriasis.

The invention provides methods of preventing, treating or ameliorating an autoimmune disorder or an inflammatory disorder or one or more symptoms thereof, said methods comprising administering to a subject in need thereof a dose of a prophylactically or therapeutically effective amount of one or more CD2 binding molecules, wherein administration of said dose results in CD2 binding molecules binding to at least 25%, at least 30%, at least 35%, at least 40%, at least 45%, at least 50%, at least 55%, at least 60%, at least 65%, at least 70%, at least 75%, at least 75%, at least 80%, at least 85% or at least 90% of the CD2 polypeptides expressed by peripheral blood lymphocytes. Preferably, a subsequent dose is administered to said subject when the percentage of CD2 polypeptides bound to CD2 binding molecules drops to 20% or less, 15% or less, or 10% or less.

The present invention provides methods of preventing, treating or ameliorating an autoimmune disorder or an inflammatory disorder or one or more symptoms thereof, said methods comprising administering to a subject in need thereof a first dose of a prophylactically or therapeutically effective amount of a CD2 binding molecule and administering to said subject one or more subsequent doses of a prophylactically or therapeutically effective amount of the CD2 binding molecule after administration of said first dose, wherein administration of said first dose results in 25% to 90% of the CD2 polypeptides expressed by peripheral blood lymphocytes being bound to CD2 binding molecules and administration of said subsequent doses restore 25% to 90% of the CD2 polypeptides expressed by peripheral blood lymphocytes being bound by CD2 binding molecules. In a preferred embodiment, the CD2 binding molecule is MEDI-507 or an antigen-binding fragment thereof.

The invention provides methods of preventing, treating or ameliorating an autoimmune disorder or an inflammatory disorder or one or more symptoms thereof, said methods comprising: (a) administering to a subject in need thereof one or more doses of a prophylactically or therapeutically effective amount of one or more CD2 binding molecules; and (b) monitoring the percentage of CD2 polypeptides expressed by peripheral blood lymphocytes bound by CD2 binding molecules in said subject after administration of a certain number of doses and prior to the administration of a subsequent dose. The mean absolute lymphocyte count in the subject may be determined after 1, 2, 3, 4, 5, 6, 7, 8, 9, 10, 11, 12, 13, 14 or more doses of the CD2 binding molecules. Preferably, the administration of one or more subsequent doses of a prophylactically or therapeutically effective amount of one or more CD2 binding molecules is based upon whether the percentage of CD2 polypeptides bound to a CD2 binding molecule is within the range of 25% to 90%.

The invention provides methods of preventing, treating or ameliorating an autoimmune disorder or an inflammatory disorder or one or more symptoms thereof, said method comprising: (a) administering to a subject in need thereof one or more doses of a prophylactically or therapeutically effective amount of a CD2 binding molecule; (b) assessing the percentage of CD2 polypeptides bound by CD2 binding molecules after administration of one or more of said doses and prior to the administration of a subsequent dose; and (c) administering to said subject one or more subsequent doses of a prophylactically or therapeutically effective amount of the CD2 binding molecule when the percentage of CD2 polypeptides expressed by peripheral blood lymphocytes bound by CD2 binding molecules is approximately 20% or less, approximately 15% or less, approximately 10% or less, or approximately 5% or less. In a preferred embodiment, the CD2 binding molecule is MEDI-507 or an antigen-binding fragment thereof.

The invention provides methods of preventing, treating or ameliorating an autoimmune disorder or an inflammatory disorder or one or more symptoms thereof, said methods comprising: (a) administering to a subject in need thereof one or more doses of a prophylactically or therapeutically effective amount of one or more CD2 binding molecules; (b) monitoring the percentage of CD2 polypeptides bound by CD2 binding molecules after administration of one or more of said doses and prior to the administration of a subsequent dose; and (c) maintaining a 25% to 90% receptor occupancy by said CD2 binding molecules in said subject by repeating step (a) as necessary.

The invention provides methods of preventing, treating or ameliorating psoriasis or one or more symptoms thereof, said methods comprising administering to a subject in need thereof one or more doses of a prophylactically or therapeutically effective amount of one or more CD2 binding molecules, wherein administration of said doses results in a mean absolute lymphocyte count of approximately 500 cells/μl to below 1200 cells/μl. Preferably, the administration of said doses results in at least a 10%, preferably 15%, 20%, 25%, 30%, 35%, 40%, 45%, 50%, 55%, 60%, 65%, 70%, 75% or more reduction of said subject's Psoriasis Area and Severity Index (PASI) score or a 10%, 15%, 20%, 25%, 30%, 35%, 40%, 45%, 50%, 55%, 60%, 65%, 70%, 75% or greater improvement in the subject's quality of life.

The invention provides methods of preventing, treating or ameliorating psoriasis or one or more symptoms thereof said methods comprising administering to a subject in need thereof one or more doses of a prophylactically or therapeutically effective amount of one or more CD2 binding molecules, wherein administration of said doses results in at least 25%, at least 30%, at least 35%, at least 40%, at least 45%, at least 50%, at least 55%, at least 60%, at least 65%, at least 70%, at least 75% or at least 80% of CD2 polypeptides expressed by peripheral blood lymphocytes being bound by CD2 binding molecules. Preferably, the administration of said doses results in at least a 10%, preferably 15%, 20%, 25%, 30%, 35%, 40%, 45%, 50%, 55%, 60%, 65%, 70%, 75% or more reduction of said subject's Psoriasis Area and Severity Index (PASI) score or a 10%, 15%, 20%, 25%, 30%, 35%, 40%, 45%, 50%, 55%, 60%, 65%, 70%, 75% or greater improvement in the subject's quality of life.

The invention provides methods of preventing, treating or ameliorating psoriasis in a human which avoids or reduces adverse effects associated with decreasing lymphocyte counts, said methods comprising administering doses of a prophylactically or therapeutically effective amount of one or more CD2 binding molecules, said doses being effective to achieve a reduction in said human's PASI score by at least 25%, at least 15%, at least 20%, at least 25%, at least 30%, at least 35%, at least 40%, at least 45%, at least 50%, at least 55%, at least 60%, at least 65%, at least 70%, at least 75% or more reduction of said subject's Psoriasis Area and Severity Index (PASI) score, but insufficient to cause a reduction in lymphocyte count to below 500 cells/μl. Preferably, the mean absolute lymphocyte count is between 500 cells/μl and 1200 cells/μl.

The invention provides methods of preventing, treating or ameliorating psoriasis or one or more symptoms thereof, said methods comprising administering to a subject in need thereof one or more doses of a prophylactically or therapeutically effective amount of MEDI-507. In a preferred embodiment, the invention provides a method of preventing, treating or ameliorating psoriasis or one or more symptoms thereof, said method comprising administering to a subject in need thereof one or more doses of a prophylactically or therapeutically effective amount of MEDI-507, wherein administration of said doses results in a lymphocyte count of approximately 500 cells/μl, preferably approximately 550 cells/μl, approximately 600 cells/μl, approximately 650 cells/μl, approximately 700 cells/μl, approximately 750 cells/μl, approximately 800 cells/μl, approximately 850 cells/μl, approximately 900 cells/μl, approximately 1000 cells/μl, approximately 1050 cells/μl, approximately 1100 cells/μl, approximately 1150 cells/μl, approximately 1200 cells/μl or approximately 1250 cells/μl. In another preferred embodiment, the invention provides a method of preventing, treating or ameliorating psoriasis or one or more symptoms thereof, said method comprising administering to a subject in need thereof one or more doses of a prophylactically or therapeutically effective amount of MEDI-507, wherein administration of said doses results in at least 25%, at least 30%, at least 35%, at least 40%, at least 45%, at least 50%, at least 55%, at least 60%, at least 65%, at least 70%, at least 75% or at least 80% of CD2 polypeptides expressed by peripheral blood lymphocytes being bound by MEDI-507. In accordance with these embodiments, the administration of said doses results in at least a 10%, preferably 15%, 20%, 25%, 30%, 35%, 40%, 45%, 50%, 55%, 60%, 65%, 70%, 75% or more reduction of said subject's PASI score or a 10%, 15%, 20%, 25%, 30%, 35%, 40%, 45%, 50%, 55%, 60%, 65%, 70%, 75% or greater improvement in the subject's quality of life.

The invention provides pharmaceutical compositions for use in accordance with the methods of the invention, said pharmaceutical compositions comprising one or more CD2 antagonists and a pharmaceutically acceptable carrier. In a specific embodiment, the invention provides a pharmaceutical composition for use in accordance with the methods of the invention, said pharmaceutical composition comprising one or more CD2 binding molecules. In accordance with this embodiment, the CD2 binding molecule may or may not be a fusion protein that immunospecifically binds to a CD2 polypeptide. In another embodiment, the invention provides a pharmaceutical composition for use in accordance with the methods of the invention comprising one or more fusion proteins that immunospecifically bind to CD2 polypeptides. In another embodiment, the invention provides a pharmaceutical composition for use in accordance with the methods of the invention comprising one or more antibodies that immunospecifically bind to CD2 polypeptides. In a preferred embodiment, the invention provides a pharmaceutical composition for use in accordance with the methods of the invention, said pharmaceutical composition comprising MEDI-507 or an antigen-binding fragment thereof.

The present invention provides article of manufactures comprising packaging material and a pharmaceutical composition of the invention in suitable form for administration to a subject contained within said packaging material. In particular, the present invention provides article of manufactures comprising packaging material and a pharmaceutical composition of the invention in suitable form for administration to a subject contained within said packaging material wherein said pharmaceutical composition comprises one or more CD2 binding molecules, one or more prophylactic or therapeutic agents other than CD2 binding molecules, and a pharmaceutically acceptable carrier. The articles of manufacture of the invention may include instructions regarding the use or administration of a pharmaceutical composition, or other informational material that advises the physician, technician or patient on how to appropriately prevent or treat the disease or disorder in question.

In a specific embodiment, an article of manufacture comprises packaging material and a pharmaceutical agent contained within said packaging material, wherein said pharmaceutical agent comprises a CD2 binding molecule and a pharmaceutically acceptable carrier, wherein said article of manufacture includes instruction means indicating a dosing regimen comprising administering an initial dosing, and optionally administering a subsequent dose or doses, of said pharmaceutical agent to a subject suffering from one or more symptoms associated with an autoimmune disorder or an inflammatory disorder, wherein the instruction means suggests a dosing regimen comprising an initial dosing that results in CD2 binding molecules binding to at least 30% of the CD2 molecules expressed by the subject's peripheral blood lymphocytes for at least 1 hour after the administration of said initial dosing, and wherein the instruction means suggests a dosing interval for said dosing regimen such that any dose/doses administered subsequent to said initial dosing, if administered, is/are only administered when 20% or less of the CD2 molecules expressed by peripheral blood lymphocytes are bound by previously administered CD2 binding molecules. In another embodiment, an article of manufacture comprises packaging material and a pharmaceutical composition in suitable form for administration to a human contained within said packaging material, wherein said pharmaceutical composition comprises MEDI-507 or an antigen-binding fragment thereof, and a pharmaceutically acceptable carrier.

4.1. CD2 Antagonists

CD2 antagonists include, but are not limited to, proteinaceous molecules (e.g., proteins, polypeptides, peptides, fusion proteins, antibodies, and antibody fragments), nucleic acid molecules (e.g., CD2 antisense nucleic acid molecules, triple helices or nucleic acid molecules encoding proteinaceous molecules), organic molecules, inorganic molecules, small organic molecules, drugs, and small inorganic molecules that block, inhibit, reduce or neutralize a function, an activity and/or the expression of a CD2 polypeptide. In various embodiments, a CD2 antagonist reduces the function, activity and/or expression of a CD2 polypeptide by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% relative to a control such as PBS.

In certain embodiments, CD2 antagonists directly or indirectly the depletion of peripheral blood lymphocytes, preferably T lymphocytes and/or NK cells. In other embodiments, a CD2 antagonist inhibits T-cell proliferation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% in an in vivo or in vitro assay described herein or known to one of skill in the art. In other embodiments, a CD2 antagonist induces cytolysis of T-cells. In other embodiments, a CD2 antagonist inhibits T-cell proliferation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% and inducing cytolysis of peripheral blood T-cells in an in vivo or in vitro assay described herein or known to one of skill in the art. In yet other embodiments, a CD2 binding antagonist inhibits T-cell activation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% in an in vivo or in vitro assay described herein or known to one of skill in the art.

In certain embodiments a CD2 antagonist inhibits or reduces the interaction between a CD2 polypeptide and LFA-3 by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% in an in vivo or in vitro assay described herein (e.g., an ELISA) or known to one of skill in the art. In other embodiments, a CD2 antagonist does not inhibit the interaction between a CD2 polypeptide and LFA-3. In yet other embodiments, a CD2 antagonist inhibits the interaction between a CD2 polypeptide and LFA-3 by less than 20%, less 15%, less than 10%, or less than 5%.

In certain embodiments, a CD2 antagonist does not induce or reduces cytokine expression and/or release in an in vivo or in vitro assay described herein or well-known to one of skill in the art. In a specific embodiment, a CD2 antagonist does not induce an increase in the concentration of cytokines such as, e.g., interferon-y (“IFN-γ”), interleukin-2 (“IL-2”), interleukin-4 (“IL-4”), interleukin-6 (“IL-6”), interleukin-9 (“IL-9”), interleukin-12 (“IL-12”), and interleukin-15 (“IL-15”) in the serum of a subject administered a CD2 antagonist. In alternative embodiments, a CD2 antagonist induces cytokine expression and/or release in an in vitro or in vivo assay described herein or known to one of skill in the art. In a specific embodiment, a CD2 antagonist induces an increase in the concentration of cytokines such as, e.g., IFN-γ, IL-2, IL4, IL-6, interleukin-7 (“IL-7”), IL-9, interleukin-10 (“IL-10”), and tumor necrosis factor α (“TNF-α”) in the serum of a subject administered a CD2 binding molecule. Serum concentrations of cytokines can be measured by any technique well-known to one of skill in the art such as immunoassays, including, e.g., ELISA.

In certain embodiments, a CD2 antagonist induces T-cell anergy in an in vivo or in vitro assay described herein or known to one of skill in the art. In alternative embodiments, a CD2 antagonist does not induce T-cell anergy in an in vivo or in vitro assay described herein or known to one of skill in the art. In other embodiments, a CD2 antagonist elicits a state of antigen-specific unresponsiveness or hyporesponsiveness for at least 30 minutes, at least 1 hour, at least 2 hours, at least 6 hours, at least 12 hours, at least 24 hours, at least 2 days, at least 5 days, at least 7 days, at least 10 days or more in an in vitro assay described herein or well-known to one of skill in the art.

In other embodiments, a CD2 antagonist inhibits T-cell activation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% and inhibits T-cell proliferation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% in an in vivo or in vitro assays described herein or well-known to one of skill in the art.

In certain embodiments, a CD2 antagonist is not a small organic molecule. In other embodiments, a CD2 antagonist is not an antisense nucleic acid molecule or triple helix. In a preferred embodiment, a CD2 antagonist is a CD2 binding molecule.

In a preferred embodiment, proteins, polypeptides or peptides (including antibodies and fusion proteins) that are utilized as CD2 antagonists are derived from the same species as the recipient of the proteins, polypeptides or peptides so as to reduce the likelihood of an immune response to those proteins, polypeptides or peptides. In another preferred embodiment, when the subject is a human, the proteins, polypeptides, or peptides that are utilized as CD2 antagonists are human or humanized.

Nucleic acid molecules encoding proteins, polypeptides, or peptides that function as CD2 antagonists, or proteins, polypeptides, or peptides that function as CD2 antagonists can be administered to a subject with an inflammatory or autoimmune disorder in accordance with the methods of the invention. Further, nucleic acid molecules encoding derivatives, analogs, fragments or variants of proteins, polypeptides, or peptides that function as CD2 antagonists, or derivatives, analogs, fragments or variants of proteins, polypeptides, or peptides that function as CD2 antagonists can be administered to a subject with an inflammatory or autoimmune disorder in accordance with the methods of the invention. Preferably, such derivatives, analogs, variants and fragments retain the CD2 antagonist activity of the full-length wild-type protein, polypeptide, or peptide.

4.2. CD2 Binding Molecules

The present invention encompasses the use of CD2 binding molecules for the prevention, treatment or amelioration an autoimmune disorder or an inflammatory disorder in a subject. In particular, present invention encompasses the use of CD2 binding molecules for the prevention, treatment or amelioration of one or more symptoms associated with psoriasis.

The term “CD2 binding molecule” and analogous terms, as used herein, refer to a bioactive molecule that immunospecifically binds to a CD2 polypeptide and directly or indirectly modulate an activity or function of lymphocytes, in particular, peripheral blood T-cells. In a specific embodiment, CD2 binding molecules directly or indirectly mediate the depletion of lymphocytes, in particular peripheral blood T-cells. Preferably, the CD2 binding molecule binds to a CD2 polypeptide and preferentially mediates depletion of memory T cells (i.e., CD45RO⁺ T cells) and not naive T cells. In a specific embodiment, a CD2 binding molecule immunospecifically binds a CD2 polypeptide expressed by an immune cell such as a T-cell or NK cell. In a preferred embodiment, a CD2 binding molecule immunospecifically binds a CD2 polypeptide expressed by a T-cell and/or NK cell. CD2 binding molecules can be identified, for example, by immunoassays or other techniques well-known to those of skill in the art. CD2 binding molecules include, but are not limited to, peptides, polypeptides, fusion proteins, small molecules, mimetic agents, synthetic drugs, organic molecules, inorganic molecules, and antibodies.

In one embodiment, a CD2 binding molecule mediates depletion of peripheral blood T-cells by inhibiting T-cell proliferation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% in an in vivo or in vitro assay described herein or known to one of skill in the art. In another embodiment, a CD2 binding molecule mediates depletion of peripheral blood T-cells by inducing cytolysis of T-cells. In yet another embodiment, a CD2 binding molecule mediates depletion of peripheral blood T-cells by inhibiting T-cell proliferation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% and inducing cytolysis of peripheral blood T-cells in an in vivo or in vitro assay described herein or known to one of skill in the art.

In a specific embodiment, a CD2 binding molecule immunospecifically binds to a CD2 polypeptide and does not non-specifically bind to other polypeptides. In another embodiment, a CD2 binding molecule immunospecifically binds to a CD2 polypeptide and has cross-reactivity with other antigens. In a preferred embodiment, a CD2 binding molecule immunospecifically binds to a CD2 polypeptide and does not cross-react with other antigens.

In one embodiment, a CD2 binding molecule inhibits or reduces the interaction between a CD2 polypeptide and a naturally occurring in vivo CD2 binding partner (e.g., an LFA-3 molecule) by approximately 25%, 30%, 35%, 40%, 45%, 50%, 55%, 60%, 65%, 70%, 75%, 80%, 85%, 90%, 95%, or 98% in an in vivo or in vitro assay described herein or well-known to one of skill in the art. In an alternative embodiment, a CD2 binding molecule does not inhibit the interaction between a CD2 polypeptide and a naturally occurring in vivo CD2 binding partner (e.g., LFA-3 molecule) in an in vivo or in vitro assay described herein or known to one of skill in the art. In another embodiment, a CD2 binding molecule inhibits the interaction between a CD2 polypeptide and LFA-3 by less than 20%, less than 15%, less than 10%, or less than 5%. A naturally occurring in vivo CD2 binding partner includes, but is not limited to, a peptide, a polypeptide, and an organic molecule that binds to a CD2 polypeptide. Preferably, a naturally occurring in vivo CD2 binding partner binds to the extracellular domain or a fragment thereof of a CD2 polypeptide.

In a specific embodiment, a CD2 binding molecule inhibits T-cell activation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% in an in vivo or in vitro assay described herein or known to one of skill in the art.

In another embodiment, a CD2 binding molecule does not induce or reduces cytokine expression and/or release in an in vivo or in vitro assay described herein or well-known to one of skill in the art. In a specific embodiment, a CD2 binding molecule does not induce an increase in the concentration of cytokines such as, e.g., interferon-γ (“IFN-γ”), interleukin-2 (“IL-2”), interleukin-4 (“IL-4”), interleukin-6 (“IL-6”), interleukin-9 (“IL-9”), interleukin-12 (“IL-12”), and interleukin-15 (“IL-15”) in the serum of a subject administered a CD2 binding molecule. In an alternative embodiment, a CD2 binding molecule induces cytokine expression and/or release in an in vitro or in vivo assay described herein or known to one of skill in the art. In a specific embodiment, a CD2 binding molecule induces an increase in the concentration of cytokines such as, e.g., IFN-γ, IL-2, IL4, IL-6, interleukin-7 (“IL-7”), IL-9, interleukin-10 (“IL-10”), and tumor necrosis factor α (“TNF-α”) in the serum of a subject administered a CD2 binding molecule. Serum concentrations of cytokines can be measured by any technique well-known to one of skill in the art such as immunoassays, including, e.g., ELISA.

In a specific embodiment, a CD2 binding molecule induces T-cell anergy in an in vivo or in vitro assay described herein or known to one of skill in the art. In an alternative embodiment, a CD2 binding molecule does not induce T-cell anergy in an in vivo or in vitro assay described herein or known to one of skill in the art. In another embodiment, a CD2 binding molecule elicits a state of antigen-specific unresponsiveness or hyporesponsiveness for at least 30 minutes, at least 1 hour, at least 2 hours, at least 6 hours, at least 12 hours, at least 24 hours, at least 2 days, at least 5 days, at least 7 days, at least 10 days or more in an in vitro assay described herein or well-known to one of skill in the art.

In another embodiment, a CD2 binding molecule inhibits T-cell activation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% and inhibits T-cell proliferation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% in an in vivo or in vitro assays described herein or well-known to one of skill in the art.

In one embodiment, a CD2 binding molecule is an antibody or antigen-binding fragment thereof that immunospecifically binds to a CD2 polypeptide. In a preferred embodiment, a CD2 binding molecule is an antibody or an antigen-binding fragment thereof that immunospecifically binds to a CD2 polypeptide expressed by an immune cell such as a T-cell or NK cell. In another embodiment, a CD2 binding molecule is a peptide, a mimetic agent, an inorganic molecule or an organic molecule that immunospecifically binds to a CD2 polypeptide. In another embodiment, a CD2 binding molecule is an LFA-3 peptide, polypeptide, derivative, or analog thereof that immunospecifically binds to a CD2 polypeptide. In another embodiment, a CD2 binding molecule is a fusion protein that immunospecifically binds to a CD2 polypeptide. In a preferred embodiment, a CD2 binding molecule is a fusion protein that immunospecifically binds to a CD2 polypeptide expressed by an immune cell such as a T-cell or NK cell. In certain embodiments, a CD2 binding molecule is a small organic molecule. In other embodiments, a CD2 binding molecule is not a small organic molecule.

4.2.1. Antibodies that Immunospecifically Bind to CD2 Polypeptides

It should be recognized that antibodies that immunospecifically bind to a CD2 polypeptide are known in the art. Examples of known antibodies that immunospecifically bind to a CD2 polypeptide include, but are not limited to, the murine monoclonal antibody produced by the cell line UMCD2 (Ancell Immunology Research Products, Bayport, Minn.; Kozarsky et al., 1993, Cell Immunol. 150:235-246), the murine monoclonal antibody produced by cell line RPA2. 10 (Zymed Laboratories, Inc., San Francisco, Calif.; Rabinowitz et al., Clin. Immunol. & Immunopathol. 76(2):148-154), the rat monoclonal antibody LO-CD2b (International Publication No. WO 00/78814 A2), the rat monoclonal antibody LO-CD2a/BTI-322 (Latinne et al., 1996, Int. Immunol. 8(7):1 113-1119), and the humanized monoclonal antibody MEDI-507 (MedImmune, Inc., Gaithersburg, Md.; Branco et al., 1999, Transplantation 68(10):1588-1596).

The present invention provides antibodies that immunospecifically bind to a CD2 polypeptide expressed by an immune cell such as a T-cell or NK cell, and said antibodies modulate an activity or function of lymphocytes, preferably peripheral blood T-cells. In a specific embodiment, antibodies that immunospecifically bind to a CD2 polypeptide directly or indirectly meditate the depletion of lymphocytes, preferably peripheral blood T-cells. In particular, the present invention provides antibodies that immunospecifically bind to a CD2 polypeptide expressed by a T-cell and/or NK cell, and said antibodies mediate depletion of peripheral blood T-cells.

In a specific embodiment, antibodies that immunospecifically bind to a CD2 polypeptide inhibit or reduce the interaction between a CD2 polypeptide and LFA-3 by approximately 25%, 30%, 35%, 45%, 50%, 55%, 60%, 65%, 70%, 75%, 80%, 85%, 90%, 95%, or 98% in an in vivo or in vitro assay described herein or well-known to one of skill in the art. In an alternative embodiment, antibodies that immunospecifically bind to a CD2 polypeptide do not inhibit the interaction between a CD2 polypeptide and LFA-3 in an in vivo or in vitro assay described herein or well-known to one of skill in the art. In another embodiment, antibodies that immunospecifically bind to a CD2 polypeptide inhibit the interaction between a CD2 polypeptide and LFA-3 by less than 20%, less than 15%, less than 10%, or less than 5%.

In a specific embodiment, antibodies that immunospecifically bind to a CD2 polypeptide inhibit T-cell activation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% in an in vivo or in vitro assay described herein or well-known to one of skill in the art.

In another embodiment, antibodies that immunospecifically bind to a CD2 polypeptide do not induce or reduce cytokine expression and/or release in an in vivo or in vitro assay described herein or well-known to one of skill in the art. In a specific embodiment, antibodies that immunospecifically bind to a CD2 polypeptide do not induce an increase in the concentration cytokines such as, e.g., IFN-γ, IL-2, IL-4, IL-6, IL-9, IL-12, and IL-15 in the serum of a subject administered a CD2 binding molecule. In an alternative embodiment, antibodies that immunospecifically binds to a CD2 polypeptide induce cytokine expression and/or release in an in vitro or in vivo assay described herein or well-known to one of skill in the art. In a specific embodiment, an antibody that immunospecifically binds to a CD2 polypeptide induces an increase in the concentration of cytokines such as, e.g., IFN-γ, IL-2, IL4, IL-6, IL-7, IL-9, IL-10, and TNF-α in the serum of a subject administered a CD2 binding molecule. Serum concentrations of a cytokine can be measured by any technique well-known to one of skill in the art such as, e.g., ELISA.

In another embodiment, antibodies that immunospecifically bind to a CD2 polypeptide induce T-cell anergy in an in vivo or in vitro assay described herein or well-known to one of skill in the art. In an alternative embodiment, antibodies that immunospecifically bind to a CD2 polypeptide do not induce T-cell anergy in an in vivo or in vitro assay described herein or well-known to one of skill in the art. In another embodiment, antibodies that immunospecifically bind to a CD2 polypeptide elicit a state of antigen-specific unresponsiveness or hyporesponsiveness for at least 30 minutes, at least 1 hour, at least 2 hours, at least 6 hours, at least 12 hours, at least 24 hours, at least 2 days, at least 5 days, at least 7 days, at least 10 days or more in an in vitro assay described herein or known to one of skill in the art.

In one embodiment, antibodies that immunospecifically bind to a CD2 polypeptide mediate depletion of peripheral blood T-cells by inhibiting T-cell proliferation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% in an in vivo or in vitro assays described herein or well-known to one of skill in the art. In another embodiment, antibodies that immunospecifically bind to a CD2 polypeptide mediate depletion of peripheral blood T-cells by inhibiting T-cell proliferation by inducing cytolysis of T-cells. In yet another embodiment, antibodies that immunospecifically bind to a CD2 polypeptide mediate depletion of peripheral blood T-cells by inhibiting T-cell proliferation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% and inducing cytolysis of peripheral blood T-cells in an in vivo or in vitro assay described herein or well-known to one of skill in the art.

In another embodiment, antibodies that immunospecifically bind to a CD2 polypeptide inhibit T-cell activation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% and inhibit T-cell proliferation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% in an in vivo or in vitro assay described herein or well-known to one of skill in the art.

In another embodiment, the Fc domain of an antibody that immunospecifically binds to a CD2 polypeptide binds to an Fc receptor (“FcR”) expressed by an immune cell such as an NK cell, a monocyte, and macrophage. In a preferred embodiment, the Fc domain of an antibody that immunospecifically binds to a CD2 polypeptide binds to an FcγRIII expressed by an immune cell such as an NK cell, a monocyte, and a macrophage. In another embodiment, a fragment of the Fc domain (e.g., the CH2 and/or CH3 region of the Fc domain) of an antibody that immunospecifically binds to a CD2 polypeptide binds to an FcR expressed by an immune cell such as an NK cell, a monocyte, and a macrophage.

Antibodies that immunospecifically bind to a CD2 polypeptide include, but are not limited to, monoclonal antibodies, multispecific antibodies, human antibodies, humanized antibodies, chimeric antibodies, single-chain Fvs (scFv), single chain antibodies, Fab fragments, F(ab′) fragments, disulfide-linked Fvs (sdFv), and anti-idiotypic (anti-Id) antibodies (including, e.g., anti-Id antibodies to antibodies of the invention), and epitope-binding fragments of any of the above. In particular, antibodies that immunospecifically bind to a CD2 polypeptide include immunoglobulin molecules and immunologically active portions of immunoglobulin molecules, i.e., molecules that contain an antigen binding site that immunospecifically binds to a CD2 polypeptide. The immunoglobulin molecules of the invention can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule. In a specific embodiment, the antibodies that immunospecifically bind to a CD2 polypeptide and mediate the depletion of T-cells comprise an Fc domain or a fragment thereof (e.g., the CH2, CH3, and/or hinge regions of an Fc domain). In a preferred embodiment, the antibodies that immunospecifically bind to a CD2 polypeptide and mediate the depletion of T cells comprise an Fc domain or fragment thereof that binds to an FcR, preferably an FcγRIII, expressed by an immune cell.

The antibodies that immunospecifically bind to a CD2 polypeptide may be from any animal origin including birds and mammals (e.g., human, murine, donkey, sheep, rabbit, goat, guinea pig, camel, horse, or chicken). Preferably, the antibodies of the invention are human or humanized monoclonal antibodies. Human antibodies that immunospecifically bind to a CD2 polypeptide include antibodies having the amino acid sequence of a human immunoglobulin and antibodies isolated from human immunoglobulin libraries or from mice that express antibodies from human genes.

The antibodies that immunospecifically bind to a CD2 polypeptide may be mono specific, bispecific, trispecific or of greater multispecificity. Multispecific antibodies may be specific for different epitopes of a CD2 polypeptide or may be specific for both a CD2 polypeptide as well as for a heterologous epitope, such as a heterologous polypeptide or solid support material. See, e.g., PCT publications WO 93/17715, WO 92/08802, WO 91/00360, and WO 92/05793; Tutt, et al., J. Immunol. 147:60-69(1991); U.S. Pat. Nos. 4,474,893, 4,714,681, 4,925,648, 5,573,920, and 5,601,819; and Kostelny et al., J. Immunol. 148:1547-1553 (1992).

The present invention provides for antibodies that have a high binding affinity for a CD2 polypeptide. In a specific embodiment, an antibody that immunospecifically binds to a CD2 polypeptide has an association rate constant or kₒₙ rate (antibody (Ab)+antigen

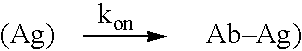

of at least 10⁵M⁻¹s⁻¹, at least 5×10⁵M⁻¹s⁻¹, at least 10⁶M⁻¹s⁻¹, at least 5×10⁻⁶M⁻¹s⁻¹, at least 10⁷M⁻¹s⁻¹, at least 5×10⁷M⁻¹s⁻¹, or at least 10⁸M⁻¹s⁻¹. In a preferred embodiment, an antibody that immunospecifically binds to a CD2 polypeptide has a kₒₙ of at least 2×10⁵M⁻¹s⁻¹, at least 5×10⁵M⁻¹s⁻¹, at least 10⁶M⁻¹s⁻¹, at least 5×10⁶M⁻¹s⁻¹, at least 10⁷M⁻¹s⁻¹, at least 5×10⁷M⁻¹s⁻¹ or at least 10⁸M⁻¹s⁻¹.

In another embodiment, an antibody that immunospecifically binds to a CD2 polypeptide has a k_{off} rate (antibody (Ab)+antigen

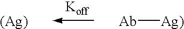

of less than 10⁻¹s⁻¹, less than 5×10⁻¹s⁻¹, less than 10⁻²s⁻¹, less than 5×10⁻²s⁻¹, less than 10⁻³s⁻¹, less than 5×10⁻³s⁻¹, less than 10⁻⁴s⁻¹, less than 5×10⁻⁴s⁻¹, less than 10⁻⁵s⁻¹, less than 5×10⁻⁵s⁻¹, less than 10⁻⁶s⁻¹, less than 5×10⁻⁶s⁻¹, less than 10⁻⁷s⁻¹, less than 5×10⁻⁷s⁻¹, less than 10⁻⁸s⁻¹, less than 5×10⁻⁸s⁻¹, less than 10⁻⁹s⁻¹, less than 5×10⁻⁹s⁻¹, or less than 10⁻¹⁰s⁻¹. In a preferred embodiment, an antibody that immunospecifically binds to a CD2 polypeptide has a kₒₙ of less than 5×10⁻⁴s⁻¹, less than 10⁻⁵s⁻¹, less than 5×10⁻⁵s⁻¹, less than 10⁻⁶s⁻¹, less than 5×10⁻⁶s⁻¹, less than 10⁻⁷s⁻¹, less than 5×10⁻⁷s⁻¹, less than 10⁻⁸s⁻¹, less than 5×10⁻⁸s⁻¹, less than 10⁻⁹s⁻¹, less than 5×10⁻⁹s⁻¹, or less than 10⁻¹⁰s⁻¹.

In another embodiment, an antibody that immunospecifically binds to a CD2 polypeptide has an affinity constant or Kₐ (kₒₙ/k_{off}) of at least 10²M⁻¹, at least 5×10²M⁻¹, at least 10³M⁻¹, at least 5×10³M⁻¹, at least 10⁴M⁻¹, at least 5×10⁴M⁻¹, at least 10⁵M⁻¹, at least 5×10⁵M⁻¹, at least 10⁶M⁻¹, at least 5×10⁶M⁻¹, at least 10⁷M⁻¹, at least 5×10⁷M⁻¹, at least 10⁸M⁻¹, at least 5×10⁸M⁻¹, at least 10⁹M⁻¹, at least 5×10⁹M⁻¹, at least 10¹⁰M⁻¹, at least 5×10¹⁰M⁻¹, at least 10¹¹M⁻¹, at least 5×10¹¹M⁻¹, at least 10¹²M⁻¹, at least 5×10¹²M⁻¹, at least 10¹³M⁻¹, at least 5×10¹³M⁻¹, at least 10¹⁴M⁻¹, at least 5×10¹⁴M⁻¹, at least 10¹⁵M⁻¹, or at least 5×10¹⁵M⁻¹. In yet another embodiment, an antibody that immunospecifically binds to a CD2 polypeptide has a dissociation constant or K_{d} (k_{off}/kₒₙ) of less than 10⁻²M, less than 5×10⁻²M, less than 10⁻³M, less than 5×10⁻³M, less than 10⁻⁴M, less than 5×10⁻⁴M, less than 10⁻⁵M, less than 5×10⁻⁵M, less than 10⁻⁶M, less than 5×10⁻⁶M, less than 10⁻⁷M, less than 5×10⁻⁷M, less than 10⁻⁸M, less than 5×10⁻⁸ M, less than 10⁻⁹M, less than 5×10⁻⁹M, less than 10⁻¹⁰M, less than 5×10¹⁰M, less than 10⁻¹¹M, less than 5×10⁻¹ M, less than 10⁻¹²M, less than 5×10⁻¹²M, less than 10⁻¹³M, less than 5×10⁻¹³M, less than 10⁻¹⁴M, less than 5×10⁻¹⁴M, less than 10⁻¹⁵M, or less than 5×10⁻¹⁵ M.

In a specific embodiment, an antibody that immunospecifically binds to a CD2 polypeptide is LO-CD2a/BTI-322 or an antigen-binding fragment thereof e.g., (one or more complementarity determining regions (CDRs) of LO-CD2a/BTI-322). LO-CD2a/BTI-322 has the amino acid sequence disclosed, e.g., in U.S. Pat. Nos. 5,730,979, 5,817,31 1, and 5,951,983; and U.S. application Ser. Nos. 09/056,072 and 09/462,140 (each of which is incorporated herein by reference in its entirety), or the amino acid sequence of the monoclonal antibody produced by the cell line deposited with the American Type Culture Collection (ATCC®), 10801 University Boulevard, Manassas, Va. 20110-2209 on Jul. 28, 1993 as Accession Number HB 11423. In an alternative embodiment, an antibody that immunospecifically binds to a CD2 polypeptide is not LO-CD2a/BTI-322 or an antigen-binding fragment of LO-CD2a/BTI-322.

In another specific embodiment, an antibody that immunospecifically binds to a CD2 polypeptide is LO-CD2b or an antigen-binding fragment thereof (e.g., one or more CDRs of LO-CD2b). LO-CD2b has the amino acid sequence of the antibody produced by the cell line deposited with the ATCC®, 10801 University Boulevard, Manassas, Va. 20110-2209 on Jun. 22, 1999 as Accession Number PTA-802, or disclosed in, e.g., Dehoux et al., 2000, Transplantation 69(12):2622-2633 and International Publication No. WO 00/78814 (each of which is incorporated herein by reference in its entirety). In an alternative embodiment, an antibody that immunospecifically binds to a CD2 polypeptide is not LO-CD2b or an antigen-binding fragment of LO-CD2b.

In a preferred embodiment, an antibody that immunospecifically binds to a CD2 polypeptide is MEDI-507 or an antibody-binding fragment thereof (e.g., one or more CDRs of MEDI-507). MEDI-507 is disclosed, e.g., in PCT Publication No. WO 99/03502 and U.S. application Ser. No. 09/462,140, each of which is incorporated herein by reference in its entirety. In an alternative embodiment, an antibody of the present invention is not MEDI-507 or an antigen-binding fragment of MEDI-507.

The present invention also provides antibodies that immunospecifically bind a CD2 polypeptide, said antibodies comprising a variable heavy (“VH”) domain having an amino acid sequence of the VH domain for LO-CD2a/BTI-322 or MEDI-507. The present invention also provides antibodies that immunospecifically bind to a CD2 polypeptide, said antibodies comprising a VH CDR having an amino acid sequence of any one of the VH CDRs listed in Table 1.

TABLE 1
CDR Sequences Of LO-CD2a/BTI-322

| CDR | Sequence | SEQ ID NO: |
|---|---|---|
| VH1 | EYYMY | 1 |
| VH2 | RIDPEDGSIDYVEKFKK | 2 |
| VH3 | GKFNYRFAY | 3 |
| VL1 | RSSQSLLHSSGNTLNW | 4 |
| VL2 | LVSKLES | 5 |
| VL3 | MQFTHYPYT | 6 |

In one embodiment, antibodies that immunospecifically bind to a CD2 polypeptide comprise a VH CDR1 having the amino acid sequence of SEQ ID NO:1. In another embodiment, antibodies that immunospecifically bind to a CD2 polypeptide comprise a VH CDR2 having the amino acid sequence of SEQ ID NO:2. In another embodiment, antibodies that immunospecifically bind to a CD2 polypeptide comprise a VH CDR3 having the amino acid sequence of SEQ ID NO:3. In a preferred embodiment, antibodies that immunospecifically bind to a CD2 polypeptide comprise a VH CDR1 having the amino acid sequence of SEQ ID NO:1, a VH CDR2 having the amino acid sequence of SEQ ID NO:2, and a VH CDR3 having the amino acid sequence of SEQ ID NO:3.

The present invention also provides antibodies that immunospecifically bind to a CD2 polypeptide, said antibodies comprising a variable light (“VL”) domain having an amino acid sequence of the VL domain for LO-CD2a/BTI-322 or MEDI-507. The present invention also provides antibodies that immunospecifically bind to a CD2 polypeptide, said antibodies comprising a VL CDR having an amino acid sequence of any one of the VL CDRs listed in Table 1.

In one embodiment, antibodies that immunospecifically bind to a CD2 polypeptide comprise a VL CDR1 having the amino acid sequence of SEQ ID NO:4. In another embodiment, antibodies that immunospecifically bind to a CD2 polypeptide comprise a VL CDR2 having the amino acid sequence of SEQ ID NO:5. In another embodiment, antibodies that immunospecifically bind to a CD2 polypeptide comprise a VL CDR3 having the amino acid sequence of SEQ ID NO:6. In a preferred embodiment, antibodies that immunospecifically bind to a CD2 polypeptide comprise a VL CDR1 having the amino acid sequence of SEQ ID NO:4, a VL CDR2 having the amino acid sequence of SEQ ID NO:5, and a VL CDR3 having the amino acid sequence of SEQ ID NO:6.

The present invention also provides antibodies that immunospecifically bind to a CD2 polypeptide, said antibodies comprising a VH domain disclosed herein combined with a VL domain disclosed herein, or other VL domain. The present invention further provides antibodies that immunospecifically bind to a CD2 polypeptide, said antibodies comprising a VL domain disclosed herein combined with a VH domain disclosed herein, or other VH domain.

The present invention also provides antibodies that immunospecifically bind to a CD2 polypeptide, said antibodies comprising one or more VH CDRs and one or more VL CDRs listed in Table 1. In particular, the invention provides for an antibody that immunospecifically binds to a CD2 polypeptide, said antibody comprising a VH CDR1 and a VL CDR1, a VH CDR1 and a VL CDR2, a VH CDR1 and a VL CDR3, a VH CDR2 and a VL CDR1, VH CDR2 and VL CDR2, a VH CDR2 and a VL CDR3, a VH CDR3 and a VH CDR1, a VH CDR3 and a VL CDR2, a VH CDR3 and a VL CDR3, or any combination thereof of the VH CDRs and VL CDRs listed in Table 1.

In one embodiment, an antibody that immunospecifically binds to a CD2 polypeptide comprises a VH CDR1 having the amino acid sequence of SEQ ID NO:1 and a VL CDR1 having the amino acid sequence of SEQ ID NO:4. In another embodiment, an antibody that immunospecifically binds to a CD2 polypeptide comprises a VH CDR1 having the amino acid sequence of SEQ ID NO:1 and a VL CDR2 having the amino acid sequence of SEQ ID NO:5. In another embodiment, an antibody that immunospecifically binds to a CD2 polypeptide comprises a VH CDR1 having the amino acid sequence of SEQ ID NO:1 and a VL CDR3 having the amino acid sequence of SEQ ID NO:6.

In another embodiment, an antibody that immunospecifically binds to a CD2 polypeptide comprises a VH CDR2 having the amino acid sequence of SEQ ID NO:2 and a VL CDR1 having the amino acid sequence of SEQ ID NO:4. In another embodiment, an antibody that immunospecifically binds to a CD2 polypeptide comprises a VH CDR2 having the amino acid sequence of SEQ ID NO:2 and a VL CDR2 having the amino acid sequence of SEQ ID NO:5. In another embodiment, an antibody that immunospecifically binds to a CD2 polypeptide comprises a VH CDR2 having the amino acid sequence of SEQ ID NO:2 and a VL CDR3 having the amino acid sequence of SEQ ID NO:6.

In another embodiment, an antibody that immunospecifically binds to a CD2 polypeptide comprises a VH CDR3 having the amino acid sequence of SEQ ID NO:3 and a VL CDR1 having the amino acid sequence of SEQ ID NO:4. In another embodiment, an antibody that immunospecifically binds to a CD2 polypeptide comprises a VH CDR3 having the amino acid sequence of SEQ ID NO:3 and a VL CDR2 having the amino acid sequence of SEQ ID NO:5. In a preferred embodiment, an antibody that immunospecifically binds to a CD2 polypeptide comprises a VH CDR3 having the amino acid sequence of SEQ ID NO:3 and a VL CDR3 having the amino acid sequence of SEQ ID NO:6.

The present invention also provides for a nucleic acid molecule, generally isolated, encoding an antibody that immunospecifically binds to a CD2 polypeptide. In a specific embodiment, an isolated nucleic acid molecule encodes an antibody that immunospecifically binds to a CD2 polypeptide, said antibody having the amino acid sequence of LO-CD2a/BTI-322, LO-CD2b, or MEDI-507.

In one embodiment, an isolated nucleic acid molecule encodes an antibody that immunospecifically binds to a CD2 polypeptide, said antibody comprising a VH domain having the amino acid sequence of the VH domain of LO-CD2a/BTI-322 or MEDI-507. In another embodiment, an isolated nucleic acid molecule encodes an antibody that immunospecifically binds to a CD2 polypeptide, said antibody comprising a VH domain having the amino acid sequence of the VH domain of the monoclonal antibody produced by the cell line deposited with the ATCC® as Accession Number HB 11423. In another embodiment, an isolated nucleic acid molecule encodes an antibody that immunospecifically binds to a CD2 polypeptide, said antibody comprising a VH CDR1 having the amino acid sequence of the VH CDR1 listed in Table 1. In another embodiment, an isolated nucleic acid molecule encodes an antibody that immunospecifically binds to a CD2 polypeptide, said antibody comprising a VH CDR2 having the amino acid sequence of the VH CDR2 listed in Table 1. In yet another embodiment, an isolated nucleic acid molecule encodes an antibody that immunospecifically binds to a CD2 polypeptide, said antibody comprising a VH CDR3 having the amino acid sequence of the VH CDR3 listed in Table 1.

In one embodiment, an isolated nucleic acid molecule encodes an antibody that immunospecifically binds to a CD2 polypeptide, said antibody comprising a VL domain having the amino acid sequence of the VL domain of LO-CD2a/BTI-322 or MEDI-507. In another embodiment, an isolated nucleic acid molecule encodes an antibody that immunospecifically binds to a CD2 polypeptide, said antibody comprising a VL domain having the amino acid sequence of the VL domain of the monoclonal antibody produced by the cell line deposited with the ATCC® as Accession Number HB 11423. In another embodiment, an isolated nucleic acid molecule encodes an antibody that immunospecifically binds to a CD2 polypeptide, said antibody comprising a VL CDR1 having the amino acid sequence of the VL CDR1 listed in Table 1. In another embodiment, an isolated nucleic acid molecule encodes an antibody that immunospecifically bind to a CD2 polypeptide, said antibody comprising a VL CDR2 having the amino acid sequence of the VL CDR2 listed in Table 1. In yet another embodiment, an isolated nucleic acid molecule encodes an antibody that immunospecifically binds to a CD2 polypeptide, said antibody comprising a VL CDR3 having the amino acid sequence of the VL CDR3 listed in Table 1.

In another embodiment, an isolated nucleic acid molecule encodes an antibody that immunospecifically binds to a CD2 polypeptide, said antibody comprising a VH domain having the amino acid sequence of the VH domain of LO-CD2a/BTI-322 or MEDI-507 and a VL domain having the amino acid sequence of the VL domain of LO-CD2a/BTI-322 or MEDI-507. In another embodiment, an isolated nucleic acid molecule encodes an antibody that immunospecifically binds to a CD2 polypeptide, said antibody comprising a VH CDR1, a VL CDR1, a VH CDR2, a VL CDR2, a VH CDR3, a VL CDR3, or any combination thereof having an amino acid sequence listed in Table 1.

The present invention also provides antibodies that immunospecifically bind to a CD2 polypeptide, said antibodies comprising derivatives of the VH domains, VH CDRs, VL domains, or VL CDRs described herein that immunospecifically bind to a CD2 polypeptide. Standard techniques known to those of skill in the art can be used to introduce mutations in the nucleotide sequence encoding an antibody of the invention, including, for example, site-directed mutagenesis and PCR-mediated mutagenesis which results in amino acid substitutions. Preferably, the derivatives include less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the original molecule. In a preferred embodiment, the derivatives have conservative amino acid substitutions are made at one or more predicted non-essential amino acid residues (i.e., amino acid residues which are not critical for the antibody to immunospecifically bind to a CD2 polypeptide). A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded antibody can be expressed and the activity of the antibody can be determined.

The present invention provides for antibodies that immunospecifically bind to a CD2 polypeptide, said antibodies comprising the amino acid sequence of LO-CD2a/BTI-322 or MEDI-507 with one or more amino acid residue substitutions in the variable light (VL) domain and/or variable heavy (VH) domain. The present invention also provides for antibodies that immunospecifically bind to a CD2 polypeptide, said antibodies comprising the amino acid sequence of LO-CD2a/BTI-322 or MEDI-507 with one or more amino acid residue substitutions in one or more VL CDRs and/or one or more VH CDRs. The antibody generated by introducing substitutions in the VH domain, VH CDRs, VL domain and/or VL CDRs of LO-CD2a/BTI-322 or MEDI-507 can be tested in vitro and in vivo, for example, for its ability to bind to a CD2 polypeptide, or for its ability to inhibit T-cell activation, or for its ability to inhibit T-cell proliferation, or for its ability to induce T-cell lysis, or for its ability to prevent, treat or ameliorate one or more symptoms associated with an autoimmune disorder or an inflammatory disorder.

In a specific embodiment, an antibody that immunospecifically binds to a CD2 polypeptide comprises a nucleotide sequence that hybridizes to the nucleotide sequence encoding the monoclonal antibody produced by the cell line deposited with the ATCC® as Accession Number HB 11423 under stringent conditions, e.g., hybridization to filter-bound DNA in 6× sodium chloride/sodium citrate (SSC) at about 45° C. followed by one or more washes in 0.2× SSC/0.1% SDS at about 50-65° C., under highly stringent conditions, e.g., hybridization to filter-bound nucleic acid in 6× SSC at about 45° C. followed by one or more washes in 0.1× SSC/0.2% SDS at about 68° C., or under other stringent hybridization conditions which are known to those of skill in the art (see, for example, Ausubel, F. M. et al., eds., 1989, Current Protocols in Molecular Biology, Vol. 1, Green Publishing Associates, Inc. and John Wiley & Sons, Inc., New York at pages 6.3.1-6.3.6 and 2.10.3).

In a specific embodiment, an antibody that immunospecifically binds to a CD2 polypeptide comprises a nucleotide sequence that hybridizes to the nucleotide sequence encoding the MEDI-507 under stringent conditions, e.g., hybridization to filter-bound DNA in 6× sodium chloride/sodium citrate (SSC) at about 45° C. followed by one or more washes in 0.2× SSC/0.1% SDS at about 50-65° C., under highly stringent conditions, e.g., hybridization to filter-bound nucleic acid in 6× SSC at about 45° C. followed by one or more washes in 0.1× SSC/0.2% SDS at about 68° C., or under other stringent hybridization conditions which are known to those of skill in the art (see, for example, Ausubel, F. M. et al., eds., 1989, Current Protocols in Molecular Biology, Vol. 1, Green Publishing Associates, Inc. and John Wiley & Sons, Inc., New York at pages 6.3.1-6.3.6 and 2.10.3).

In a specific embodiment, an antibody that immunospecifically binds to a CD2 polypeptide comprises an amino acid sequence of a VH domain or an amino acid sequence a VL domain encoded by a nucleotide sequence that hybridizes to the nucleotide sequence encoding the VH or VL domains of LO-CD2a/BTI-322 or MEDI-507 under stringent conditions, e.g., hybridization to filter-bound DNA in 6× sodium chloride/sodium citrate (SSC) at about 45° C. followed by one or more washes in 0.2× SSC/0.1% SDS at about 50-65 C, under highly stringent conditions, e.g., hybridization to filter-bound nucleic acid in 6× SSC at about 45° C. followed by one or more washes in 0.1× SSC/0.2% SDS at about 68° C., or under other stringent hybridization conditions which are known to those of skill in the art (see, for example, Ausubel, F. M. et al., eds., 1989, Current Protocols in Molecular Biology, Vol. 1, Green Publishing Associates, Inc. and John Wiley & Sons, Inc., New York at pages 6.3.1-6.3.6 and 2.10.3).

In another embodiment, an antibody that immunospecifically binds to a CD2 polypeptide comprises an amino acid sequence of a VH CDR or an amino acid sequence of a VL CDR encoded by a nucleotide sequence that hybridizes to the nucleotide sequence encoding any one of the VH CDRs or VL CDRs listed in Table 1 under stringent conditions e.g., hybridization to filter-bound DNA in 6× sodium chloride/sodium citrate (SSC) at about 45° C. followed by one or more washes in 0.2× SSC/0.1% SDS at about 50-65° C., under highly stringent conditions, e.g., hybridization to filter-bound nucleic acid in 6× SSC at about 45° C. followed by one or more washes in 0.1× SSC/0.2% SDS at about 68° C., or under other stringent hybridization conditions which are known to those of skill in the art.

In another embodiment, an antibody that immunospecifically binds to a CD2 polypeptide comprises an amino acid sequence of a VH CDR or an amino acid sequence of a VL CDR encoded by a nucleotide sequence that hybridizes to the nucleotide sequence encoding any one of VH CDRs or VL CDRs of the monoclonal antibody produced by the cell line deposited with the ATCC® as Accession Number HB 11423 under stringent conditions e.g., hybridization to filter-bound DNA in 6× sodium chloride/sodium citrate (SSC) at about 45° C. followed by one or more washes in 0.2× SSC/0.1% SDS at about 50-65° C., under highly stringent conditions, e.g., hybridization to filter-bound nucleic acid in 6× SSC at about 45° C. followed by one or more washes in 0.1× SSC/0.2% SDS at about 68° C., or under other stringent hybridization conditions which are known to those of skill in the art.

In another embodiment, an antibody that immunospecifically binds to a CD2 polypeptide comprises an amino acid sequence of a VH CDR and an amino acid sequence of a VL CDR encoded by nucleotide sequences that hybridizes to the nucleotide sequences encoding any one of the VH CDRs and VL CDRs listed in Table 1 under stringent conditions, e.g., hybridization to filter-bound DNA in 6× sodium chloride/sodium citrate (SSC) at about 45° C. followed by one or more washes in 0.2× SSC/0.1% SDS at about 50-65° C., under highly stringent conditions, e.g., hybridization to filter-bound nucleic acid in 6× SSC at about 45° C. followed by one or more washes in 0.1× SSC/0.2% SDS at about 68° C., or under other stringent hybridization conditions which are known to those of skill in the art.

In another embodiment, an antibody that immunospecifically binds to a CD2 polypeptide comprises an amino acid sequence of a VH CDR and an amino acid sequence of a VL CDR encoded by nucleotide sequences that hybridizes to the nucleotide sequences encoding the monoclonal antibody produced by the cell line deposited with the ATCC® as Accession Number HB 11423 under stringent conditions, e.g., hybridization to filter-bound DNA in 6× sodium chloride/sodium citrate (SSC) at about 45° C. followed by one or more washes in 0.2× SSC/0.1% SDS at about 50-65° C., under highly stringent conditions, e.g., hybridization to filter-bound nucleic acid in 6× SSC at about 45° C. followed by one or more washes in 0.1× SSC/0.2% SDS at about 68° C., or under other stringent hybridization conditions which are known to those of skill in the art.

In a specific embodiment, an antibody that immunospecifically binds to a CD2 polypeptide comprises an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of the monoclonal antibody produced by the cell line deposited with the ATCC® as Accession Number HB 11423. In another embodiment, an antibody that immunospecifically binds to a CD2 polypeptide comprises an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of MEDI-507.

In another embodiment, an antibody that immunospecifically binds to a CD2 polypeptide comprises an amino acid sequence of a VH domain that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the VH domain of MEDI-507. In another embodiment, an antibody that immunospecifically binds to a CD2 polypeptide comprises an amino acid sequence of a VH domain that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the VH domain of the monoclonal antibody produced by the cell line deposited with the ATCC® as Accession Number HB 11423.

In another embodiment, an antibody that immunospecifically binds to a CD2 polypeptide comprises an amino acid sequence of one or more VH CDRs that are at least 35%, at least 40%, at least 45%, at least 50%, at least 55%, at least 60%, at least 65%, at least 70%, at least 75%, at least 80%, at least 85%, at least 90%, at least 95%, or at least 99% identical to any of the VH CDRs listed in Table 1. In another embodiment, an antibody that immunospecifically binds to a CD2 polypeptide comprises an amino acid sequence of one or more VH CDRs that are at least 35%, at least 40%, at least 45%, at least 50%, at least 55%, at least 60%, at least 65%, at least 70%, at least 75%, at least 80%, at least 85%, at least 90%, at least 95%, or at least 99% identical to any of one of the VH CDRs of the monoclonal antibody produced by the cell line deposited with the ATCC® as Accession Number HB 11423.

In another embodiment, an antibody that immunospecifically binds to a CD2 polypeptide comprises an amino acid sequence of a VL domain that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the VL domain of MEDI-507 In another embodiment, an antibody that immunospecifically binds to a CD2 polypeptide comprises an amino acid sequence of a VL domain that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the VL domain of the monoclonal antibody produced by the cell line deposited with the ATCC® as Accession Number HB 11423.

In another embodiment, an antibody that immunospecifically binds to a CD2 polypeptide comprises an amino acid sequence of one or more VL CDRs that are at least 35%, at least 40%, at least 45%, at least 50%, at least 55%, at least 60%, at least 65%, at least 70%, at least 75%, at least 80%, at least 85%, at least 90%, at least 95%, or at least 99% identical to any of the VL CDRs listed in Table 1. In another embodiment, an antibody that immunospecifically binds to a CD2 polypeptide comprises an amino acid sequence of one or more VL CDRs that are at least 35%, at least 40%, at least 45%, at least 50%, at least 55%, at least 60%, at least 65%, at least 70%, at least 75%, at least 80%, at least 85%, at least 90%, at least 95%, or at least 99% identical to any of the VL CDRs of the monoclonal antibody produced by the cell line deposited with the ATCC® as Accession Number HB 11423.

The present invention encompasses antibodies that compete with an antibody described herein for binding to a CD2 polypeptide. In a specific embodiment, the present invention encompasses antibodies that compete with LO-CD2a/BTI-322 or an antigen-binding fragment thereof for binding to the CD2 polypeptide. In a specific embodiment, the present invention encompasses antibodies that compete with LO-CD2b or an antigen-binding fragment for binding to a CD2 polypeptide. In a preferred embodiment, the present invention encompasses antibodies that compete with MEDI-507 or an antigen-binding fragment thereof for binding to the CD2 polypeptide.

The present invention also encompasses VH domains that compete with the VH domain of LO-CD2a/BTI-322 or MEDI-507 for binding to a CD2 polypeptide. The present invention also encompasses VL domains that compete with a VL domain of LO-CD2a/BTI-322 or MEDI-507 for binding to a CD2 polypeptide.

The present invention also encompasses VH CDRs that compete with a VH CDR listed in Table 1 for binding to a CD2 polypeptide, or a VH CDR of the monoclonal antibody produced by the cell line deposited with the ATCC as Accession Number HB 11423 for binding to a CD2 polypeptide. The present invention also encompasses VL CDRs that compete with a VL CDR listed in Table 1 for binding to a CD2 polypeptide, or a VL CDR of the monoclonal antibody produced by the cell line deposited with the ATCC as Accession Number HB 11423 for binding to a CD2 polypeptide.

The antibodies that immunospecifically bind to a CD2 polypeptide include derivatives that are modified, i.e, by the covalent attachment of any type of molecule to the antibody such that covalent attachment. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids.

The present invention also provides antibodies that immunospecifically bind to a CD2 polypeptide, said antibodies comprising a framework region known to those of skill in the art. Preferably, the fragment region of an antibody of the invention is human. In a specific embodiment, an antibody that immunospecifically binds to a CD2 polypeptide comprises the framework region of MEDI-507.

The present invention also encompasses antibodies which immunospecifically bind to a CD2 polypeptide, said antibodies comprising the amino acid sequence of MEDI-507 with mutations (e.g., one or more amino acid substitutions) in the framework regions. In certain embodiments, antibodies which immunospecifically bind to a CD2 polypeptide comprise the amino acid sequence of MEDI-507 with one or more amino acid residue substitutions in the framework regions of the VH and/or VL domains.

The present invention also encompasses antibodies which immunospecifically bind to a CD2 polypeptide, said antibodies comprising the amino acid sequence of MEDI-507 with mutations (e.g., one or more amino acid residue substitutions) in the variable and framework regions.

The present invention also provides for fusion proteins comprising an antibody that immunospecifically binds to a CD2 polypeptide and a heterologous polypeptide. Preferably, the heterologous polypeptide that the antibody is fused to is useful for targeting the antibody to T-cells and/or NK cells.

4.2.1.1. Antibodies Having Increased Half-Lives that Immunospecifically Bind to CD2 Polypeptides

The present invention provides for antibodies that immunospecifically bind to a CD2 polypeptide which have a extended half-life in vivo. In particular, the present invention provides antibodies that immunospecifically bind to a CD2 polypeptide which have a half-life in an animal, preferably a mammal and most preferably a human, of greater than 3 days, greater than 7 days, greater than 10 days, preferably greater than 15 days, greater than 25 days, greater than 30 days, greater than 35 days, greater than 40 days, greater than 45 days, greater than 2 months, greater than 3 months, greater than 4 months, or greater than 5 months.

To prolong the serum circulation of antibodies (e.g., monoclonal antibodies, single chain antibodies and Fab fragments) in vivo, for example, inert polymer molecules such as high molecular weight polyethyleneglycol (PEG) can be attached to the antibodies with or without a multifunctional linker either through site-specific conjugation of the PEG to the—or C-terminus of the antibodies or via epsilon-amino groups present on lysine residues. Linear or branched polymer derivatization that results in minimal loss of biological activity will be used. The degree of conjugation can be closely monitored by SDS-PAGE and mass spectrometry to ensure proper conjugation of PEG molecules to the antibodies. Unreacted PEG can be separated from antibody-PEG conjugates by size-exclusion or by ion-exchange chromatography. PEG-derivatized antibodies can be tested for binding activity as well as for in vivo efficacy using methods well-known to those of skill in the art, for example, by immunoassays described herein.

Antibodies having an increased half-life in vivo can also be generated introducing one or more amino acid modifications (i.e., substitutions, insertions or deletions) into an IgG constant domain, or FcRn binding fragment thereof (preferably a Fc or hinge-Fc domain fragment). See, e.g., International Publication No. WO 98/23289; International Publication No. WO 97/34631; and U.S. Pat. No. 6,277,375, each of which is incorporated herein by reference in its entirety.

4.2.1.2. Antibody Conjugates

The present invention encompasses antibodies or antigen-binding fragments thereof that immunospecifically bind to a CD2 polypeptide recombinantly fused or chemically conjugated (including both covalently and non-covalently conjugations) to a heterologous polypeptide (or a fragment thereof, preferably at least 5, at least 10, at least 20, at least 30, at least 40, at least 50, at least 60, at least 70, at least 80, at least 90 or at least 100 contiguous amino acids of the polypeptide) to generate fusion proteins. The fusion does not necessarily need to be direct, but may occur through linker sequences. For example, antibodies may be used to target heterologous polypeptides to particular cell types (e.g., T-cells), either in vitro or in vivo, by fusing or conjugating the antibodies to antibodies specific for particular cell surface receptors such as, e.g., CD4 and CD8.

The present invention also encompasses antibodies or antigen-binding fragments thereof that immunospecifically bind to a CD2 polypeptide fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag.

The present invention further encompasses antibodies or antigen-binding fragments thereof that immunospecifically bind to a CD2 polypeptide conjugated to an agent which has a potential therapeutic benefit. An antibody or an antigen-binding fragment thereof that immunospecifically binds to a CD2 polypeptide may be conjugated to a therapeutic moiety such as a cytotoxin, e.g., a cytostatic or cytocidal agent, an agent which has a potential therapeutic benefit, or a radioactive metal ion, e.g., alpha-emitters. A cytotoxin or cytotoxic agent includes any agent that is detrimental to cells. Examples of a cytotoxin or cytotoxic agent include, but are not limited to, paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Agents which have a potential therapeutic benefit include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Further, an antibody or an antigen-binding fragment thereof that immunospecifically binds to a CD2 polypeptide may be conjugated to a therapeutic agent or drug moiety that modifies a given biological response. Agents which have a potential therapeutic benefit or drug moieties are not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, interferon-α (“IFN-α”), interferon-β (“IFN-β”), nerve growth factor (“NGF”), platelet derived growth factor (“PDGF”), tissue plasminogen activator (“TPA”), an apoptotic agent, e.g., TNF-α, TNF-β, AIM I (see, International Publication No. WO 97/33899), AIM II (see, International Publication No. WO 97/34911), Fas Ligand (Takahashi et al., 1994, J. Iminunol., 6:1567-1574), and VEGF (see, International Publication No. WO 99/23105), a thrombotic agent or an anti-angiogenic agent, e.g., angiostatin or endostatin; or, a biological response modifier such as, for example, a lymphokine (e.g., interleukin-1 (“IL-1 ”), IL-2, IL-6, IL-10, granulocyte macrophage colony stimulating factor (“GM-CSF”), and granulocyte colony stimulating factor (“G-CSF”)), or a growth factor (e.g., growth hormone (“GH”)).

Techniques for conjugating such therapeutic moieties to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985); and Thorpe et al., 1982, Immunol. Rev. 62:119-58.

An antibody or an antigen-binding fragment thereof that immunospecifically binds to a CD2 polypeptide can be conjugated to a second antibody to form an antibody heteroconjugate as described by Segal in U.S. Pat. No. 4,676,980, which is incorporated herein by reference in its entirety.

Antibodies or antigen-binding fragments thereof that immunospecifically bind to a CD2 polypeptide may be attached to solid supports, which are particularly useful for the purification of CD2+immune cells such as T-cells. Such solid supports include, but are not limited to, glass, cellulose, polyacrylamide, nylon, polystyrene, polyvinyl chloride or polypropylene.

4.2.2. LFA-3 Polypeptides that Immununospecifically Bind to CD2 Polypeptides

The present invention encompasses LFA-3 peptides, polypeptides, derivatives and analogs thereof that immunospecifically bind to a CD2 polypeptide for use in the prevention, treatment or amelioration of one or more symptoms associated with an autoimmune or inflammatory disorder. Preferably, the soluble LFA-3 polypeptides that immunospecifically bind to a CD2 polypeptide comprise at least 5, preferably at least 10, at least 20, at least 30, at least 40, at least 50, at least 60, at least 70, at least 80, at least 90 or at least 100 contiguous amino acid residues of LFA-3. Soluble LFA-3 peptides, polypeptides, derivatives, and analogs thereof that immunospecifically bind to a CD2 polypeptide can be derived from any species.

The nucleotide and/or amino acid sequences of LFA-3 can be found in the literature or public databases, or the nucleic acid and/or amino acid sequences can be determined using cloning and sequencing techniques well-known to one of skill in the art. For example, the nucleotide and amino acid sequences of human LFA-3 can be found in the GenBank databases (see, e.g., Accession Nos. E12817 and CAA29622).

In a specific embodiment, a soluble LFA-3 polypeptide that immunospecifically binds to a CD2 polypeptide consists the extracellular domain of naturally occurring LFA-3 or amino acid residues 1 to 187 of SEQ ID NO:7. In another embodiment, a soluble LFA-3 polypeptide that immunospecifically binds to a CD2 polypeptide comprises a fragment of an extracellular domain of LFA-3 (e.g., amino acid residues 1 to 92, amino acid residues 1 to 85, amino acid residues 1 to 80, amino acid residues 1 to 75, amino acid residues 1 to 70, amino acid residues 1 to 65, or amino acid residues 1 to 60 SEQ ID NO:7).

In a specific embodiment, a soluble LFA-3 polypeptide that immunospecifically >binds to a CD2 polypeptide inhibits or reduces the interaction between a CD2 polypeptide and LFA-3 by approximately 25%, 30%, 35%, 45%, 50%, 55%, 60%, 65%, 70%, 75%, 80%, 85%, 90%, 95%, or 98% in an in vivo or in vitro assay described herein or well-known to one of skill in the art. In an alternative embodiment, a soluble LFA-3 polypeptide that immunospecifically binds to a CD2 polypeptide does not inhibit the interaction between a CD2 polypeptide and LFA-3 in an in vivo or in vitro assay described herein or well-known to one of skill in the art. In another embodiment, a soluble LFA-3 polypeptide that immunospecifically binds to a CD2 polypeptide inhibits the interaction between a CD2 polypeptide and LFA-3 by less than 20%, less than 15%, less than 10%, or less than 5%.

In a specific embodiment, soluble LFA-3 polypeptides that immunospecifically bind to a CD2 polypeptide inhibit T-cell activation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% in an in vivo or in vitro assay described herein or well-known to one of skill in the art. In another embodiment, soluble LFA-3 polypeptides that immunospecifically bind to a CD2 polypeptide inhibit T-cell proliferation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% in an in vivo or in vitro assay described herein or well-known to one of skill in the art. In another embodiment, soluble LFA-3 polypeptides that immunospecifically bind to a CD2 polypeptide inhibit T-cell activation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% in an in vivo or in vitro assay described herein or well-known to one of skill in the art and inhibit T-cell proliferation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% in an in vivo or in vitro assay described herein or well-known to one of skill in the art.

In another embodiment, a soluble LFA-3 polypeptide that immunospecifically binds to a CD2 polypeptide does not induce or reduces cytokine expression and/or release in an in vivo or in vitro assay described herein or well-known to one of skill in the art. In a specific embodiment, soluble LFA-3 polypeptide that immunospecifically binds to a CD2 polypeptide does not induce an increase in the concentration cytokines such as, e.g., IFN-γ, IL-2, IL-4, IL-6, IL-9, IL-12, and IL-15 in the serum of a subject administered a CD2 binding molecule. In an alternative embodiment, a soluble LFA-3 polypeptide that immunospecifically binds to a CD2 polypeptide induces cytokine expression and/or release in an in vitro or in vivo assay described herein or well-known to one of skill in the art. In a specific embodiment, a soluble LFA-3 polypeptide that immunospecifically binds to a CD2 polypeptide induces an increase in the concentration of cytokines such as, e.g., IFN-γ, IL-2, IL4, IL-6, IL-7, IL-9, IL-10, and TNF-α in the serum of a subject administered a CD2 binding molecule. Serum concentrations of a cytokine can be measured by any technique well-known to one of skill in the art such as, e.g., ELISA.

In another embodiment, a soluble LFA-3 polypeptide that immunospecifically binds to a CD2 polypeptide induces T-cell anergy in an in vivo or in vitro assay described herein or known to one of skill in the art. In an alternative embodiment, a soluble LFA-3 polypeptide that immunospecifically binds to a CD2 polypeptide does not induce T-cell anergy in an in vivo or in vitro assay described herein or known to one of skill in the art. In another embodiment, a soluble LFA-3 polypeptide that immunospecifically binds to a CD2 polypeptide elicits a state of antigen-specific unresponsiveness or hyporesponsiveness for at least 30 minutes, at least 1 hour, at least 2 hours, at least 6 hours, at least 12 hours, at least 24 hours, at least 2 days, at least 5 days, at least 7 days, at least 10 days or more in an in vitro assay described herein or known to one of skill in the art.

In a specific embodiment, soluble LFA-3 polypeptides that immunospecifically bind to a CD2 polypeptide mediate depletion of peripheral blood T-cells by inducing cytolysis of T-cells. In another preferred embodiment, soluble LFA-3 polypeptides that immunospecifically bind to a CD2 polypeptide mediate depletion of peripheral blood T-cells by inhibiting T-cell proliferation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% and inducing cytolysis of peripheral blood T-cells in an in vivo or in vitro assay described herein or known to one of skill in the art.

The present invention provides for soluble LFA-3 polypeptides that immunospecifically bind to a CD2 polypeptide which have a extended half-life in vivo. In particular, the present invention provides soluble LFA-3 polypeptides that immunospecifically bind to a CD2 polypeptide which have a half-life in an animal, preferably a mammal and most preferably a human, of greater than 3 days, greater than 7 days, greater than 10 days, preferably greater than 15 days, greater than 25 days, greater than 30 days, greater than 35 days, greater than 40 days, greater than 45 days, greater than 2 months, greater than 3 months, greater than 4 months, or greater than 5 months.

To prolong the serum circulation of soluble LFA-3 polypeptides that immunospecifically bind to a CD2 polypeptide in vivo, for example, inert polymer molecules such as high molecular weight polyethyleneglycol (PEG) can be attached to the antibodies with or without a multifunctional linker either through site-specific conjugation of the PEG to the - or C-terminus of the soluble LFA-3 polypeptides or via epsilon-amino groups present on lysine residues. Linear or branched polymer derivatization that results in minimal loss of biological activity will be used. The degree of conjugation can be closely monitored by SDS-PAGE and mass spectrometry to ensure proper conjugation of PEG molecules to the soluble LFA-3 polypeptides. Unreacted PEG can be separated from LFA-3 polypeptide-PEG conjugates by size-exclusion or by ion-exchange chromatography. PEG-derivatized LFA-3 polypeptides can be tested for binding activity as well as for in vivo efficacy using methods well-known to those of skill in the art, for example, by immunoassays described herein.

4.2.2.1. LFA-3 CONJUGATES

The present invention also encompasses soluble LFA-3 peptides and polypeptides that immunospecifically bind to a CD2 polypeptide fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine provides for convenient purification of the soluble LFA-3 polypeptide. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag.

The present invention further encompasses soluble LFA-3 peptides and polypeptides that immunospecifically bind to a CD2 polypeptide conjugated to a therapeutic agent. A soluble LFA-3 polypeptide that immunospecifically binds to a CD2 polypeptide may be conjugated to a therapeutic moiety such as a cytotoxin, e.g., a cytostatic or cytocidal agent, an agent which has a potential therapeutic benefit, or a radioactive metal ion, e.g., alpha-emitters. A cytotoxin or cytotoxic agent includes any agent that is detrimental to cells. Examples of a cytotoxin or cytotoxic agent include, but are not limited to, paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Agents which have a potential therapeutic benefit include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Further, a soluble LFA-3 polypeptide that immunospecifically binds to a CD2 polypeptide may be conjugated to a a therapeutic agent or drug moiety that modifies a given biological response. Agents which have a potential therapeutic benefit or drug moieties are not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, IFN-α, IFN-β, nerve growth factor (“NGF”), platelet derived growth factor (“PDGF”), tissue plasminogen activator (“TPA”), an apoptotic agent, e.g., TNF-α, TNF-β, AIM I (see, International Publication No. WO 97/33899), AIM II (see, International Publication No. WO 97/34911), Fas Ligand (Takahashi et al., 1994, J. Immunol., 6:1567-1574), and VEGF (see, International Publication No. WO 99/23105), a thrombotic agent or an anti-angiogenic agent, e.g., angiostatin or endostatin; or, a biological response modifier such as, for example, a lymphokine (e.g., IL-1, IL-2, IL-6, IL-10, GM-CSF, and G-CSF), or a growth factor (e.g., GH).

4.2.3. Fusion Proteins that Immunospecifically Bind to CD2 Polypeptides

The present invention provides fusion proteins that immunospecifically bind to a CD2 polypeptide and modulate an activity or function of lymphocytes, preferably peripheral blood T-cells for use in preventing, treating or ameliorating one or more symptoms associated with an autoimmune disorder or an inflammatory disorder. Preferably, such fusion proteins directly or indirectly mediate depletion of lymphocytes, in particular peripheral blood T-cells. In particular, the present invention provides fusion proteins that immunospecifically bind to a CD2 polypeptide expressed by an immune cell such as a T-cell or NK cell and mediate depletion of lymphocytes, in particular peripheral blood T-cells.

In a specific embodiment, a fusion protein that immunospecifically binds to a CD2 polypeptide inhibits or reduces the interaction between a CD2 polypeptide and LFA-3 by approximately 25%, 30%, 35%, 45%, 50%, 55%, 60%, 65%, 70%, 75%, 80%, 85%, 90%, 95%, or 98% in an in vivo or in vitro assay described herein or well-known to one of skill in the art. In an alternative embodiment, a fusion protein that immunospecifically binds to a CD2 polypeptide does not inhibit the interaction between a CD2 polypeptide and LFA-3 in an in vivo or in vitro assay described herein or well-known to one of skill in the art. In another embodiment, a fusion protein that immunospecifically binds to a CD2 polypeptide inhibits the interaction between a CD2 polypeptide and LFA-3 by less than 20%, less than 15%, less than 10%, or less than 5%.

In a specific embodiment, fusion proteins that immunospecifically bind to a CD2 polypeptide inhibit T-cell activation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% in an in vivo or in vitro assay described herein or known to one of skill in the art.

In another embodiment, a fusion protein that immunospecifically binds to a CD2 polypeptide does not induce or reduces cytokine expression and/or release in an in vivo or in vitro assay described herein or well-known to one of skill in the art. In a specific embodiment, fusion protein that immunospecifically binds to a CD2 polypeptide does not induce an increase in the concentration cytokines such as, e.g., IFN-γ, IL-2, IL-4, IL-6, IL-9, IL-12, and IL-15 in the serum of a subject administered a CD2 binding molecule. In an alternative embodiment, a fusion protein that immunospecifically binds to a CD2 polypeptide induces cytokine expression and/or release in an in vitro or in vivo assay described herein or well-known to one of skill in the art. In a specific embodiment, a fusion protein that immunospecifically binds to a CD2 polypeptide induces an increase in the concentration of cytokines such as, e.g., IFN-γ, IL-2, IL4, IL-6, IL-7, IL-9, IL-10, and TNF-α in the serum of a subject administered a CD2 binding molecule. Serum concentrations of a cytokine can be measured by any technique well-known to one of skill in the art such as, e.g., ELISA.

In another embodiment, a fusion protein that immunospecifically binds to a CD2 polypeptide induces T-cell anergy in an in vivo or in vitro assay described herein or well-known to one of skill in the art. In an alternative embodiment, a fusion protein that immunospecifically binds to a CD2 polypeptide does not induce T-cell anergy in an in vivo or in vitro assay described herein or well-known to one of skill in the art. In another embodiment, a fusion protein that immunospecifically binds to a CD2 polypeptide elicits a state of antigen-specific unresponsiveness or hyporesponsiveness for at least 30 minutes, at least 1 hour, at least 2 hours, at least 6 hours, at least 12 hours, at least 24 hours, at least 2 days, at least 5 days, at least 7 days, at least 10 days or more in an in vitro assay described herein or well-known to one of skill in the art.

In a specific embodiment, fusion proteins that immunospecifically bind to a CD2 polypeptide mediate depletion of peripheral blood T-cells by inhibiting T-cell proliferation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% in an in vivo or in vitro assays described herein or well-known to one of skill in the art. In a preferred, fusion proteins that immunospecifically bind to a CD2 polypeptide mediate depletion of peripheral blood T-cells by inducing cytolysis of T-cells. In another preferred embodiment, fusion proteins that immunospecifically bind to a CD2 polypeptide mediate depletion of peripheral blood T-cells by inhibiting T-cell proliferation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% and inducing cytolysis of peripheral blood T-cells in an in vivo or in vitro assay described herein or well-known to one of skill in the art.

In another embodiment, fusion proteins that immunospecifically bind to a CD2 polypeptide inhibit T-cell activation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% and inhibit T-cell proliferation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% in an in vivo or in vitro assay described herein or known to one of skill in the art.

In another embodiment, a fusion protein that immunospecifically binds to a CD2 polypeptide binds to an FcR expressed by an immune cell such as an NK cell, a monocyte, and macrophage. In a preferred embodiment, a fusion protein that immunospecifically binds to a CD2 polypepitde binds to an FcγRIII expressed by an immune cell such as an NK cell, a monocyte, and a macrophage.

In one embodiment, a fusion protein that immunospecifically binds to a CD2 polypeptide comprises a bioactive molecule fused to the Fc domain of an immunoglobulin molecule or a fragment thereof. In another embodiment, a fusion protein that immunospecifically binds to a CD2 polypeptide comprises a bioactive molecule fused to the CH2 and/or CH3 region of the Fc domain of an immunoglobulin molecule. In yet another embodiment, a fusion protein that immunospecifically binds to a CD2 polypeptide comprises a bioactive molecule fused to the CH2, CH3, and hinge regions of the Fc domain of an immunoglobulin molecule. In accordance with these embodiments, the bioactive molecule immunospecifically binds to a CD2 polypeptide. Bioactive molecules that immunospecifically bind to a CD2 polypeptide include, but are not limited to, peptides, polypeptides, small molecules, mimetic agents, synthetic drugs, inorganic molecules, and organic molecules. Preferably, a bioactive molecule that immunospecifically binds to a CD2 polypeptide is a polypeptide comprising at least 5, preferably at least 10, at least 20, at least 30, at least 40, at least 50, at least 60, at least 70, at least 80, at least 90 or at least 100 contiguous amino acid residues, and is heterologous to the amino acid sequence of the Fc domain of an immunoglobulin molecule or a fragment thereof.

In a specific embodiment, a fusion protein that immunospecifically binds to a CD2 polypeptide comprises LFA-3 or a fragment thereof which immunospecifically binds to a CD2 polypeptide fused to the Fc domain of an immunoglobulin molecule or a fragment thereof. In another embodiment, a fusion protein that immunospecifically binds to a CD2 polypeptide comprises LFA-3 or a fragment thereof which immunospecifically binds to a CD2 polypeptide fused to the CH2 and/or CH3 region of the Fc domain of an immunoglobulin molecule. In another embodiment, a fusion protein that immunospecifically binds to a CD2 polypeptide comprises LFA-3 or a fragment thereof which immunospecifically binds to a CD2 polypeptide fused to the CH2, CH3, and hinge regions of the Fc domain of an immunoglobulin molecule.

In another embodiment, a fusion protein that immunospecifically binds to a CD2 polypeptide comprises an extracellular domain of LFA-3 (e.g., amino acid residues 1 to 187 of SEQ ID NO:7) fused to the Fc domain of an immunoglobulin molecule or a fragment thereof. In another embodiment, a fusion protein that immunospecifically binds to a CD2 polypeptide comprises an extracellular domain of LFA-3 (e.g., amino acid residues 1 to 187 of SEQ ID NO:7) fused to the CH2 and/or CH3 region of the Fc domain of an immunoglobulin molecule. In another embodiment, a fusion protein that immunospecifically binds to a CD2 polypeptide comprises an extracellular domain of LFA-3 (e.g., amino acid residues 1 to 187 of SEQ ID NO:7) fused to the CH2, CH3, and hinge regions of the Fc domain of an immunoglobulin molecule.

In another embodiment, a fusion protein that immunospecifically binds to a CD2 polypeptide comprises a fragment of an extracellular domain of LFA-3 (e.g., amino acid residues 1 to 92, amino acid residues 1 to 85, amino acid residues 1 to 80, amino acid residues 1 to 75, amino acid residues 1 to 70, amino acid residues 1 to 65, or amino acid residues 1 to 60 SEQ ID NO:7) fused to the Fc domain of an immunoglobulin molecule or a fragment thereof. In another embodiment, a fusion protein that immunospecifically binds to a CD2 polypeptide comprises a fragment of an extracellular domain of LFA-3 (e.g., amino acid residues 1 to 92, amino acid residues 1 to 85, amino acid residues 1 to 80, amino acid residues 1 to 75, amino acid residues 1 to 70, amino acid residues 1 to 65, or amino acid residues 1 to 60 SEQ ID NO:7) fused to the CH2 and/or CH3 region of the Fc domain of an immunoglobulin molecule. In another embodiment, a fusion protein that immunospecifically binds to a CD2 polypeptide comprises a fragment of an extracellular domain of LFA-3 (e.g., amino acid residues 1 to 92, amino acid residues 1 to 85, amino acid residues 1 to 80, amino acid residues 1 to 75, amino acid residues 1 to 70, amino acid residues 1 to 65, or amino acid residues 1 to 60 SEQ ID NO:7) fused to the CH2, CH3, and hinge regions of the Fc domain of an immunoglobulin molecule.

In a specific embodiment, a CD2 binding molecule is LFA-3TIP (Biogen, Inc., Cambridge, Mass.). In an alterative embodiment, a CD2 binding molecule is not LFA-3TIP.

In another embodiment, a fusion protein that immunospecifically binds to a CD2 polypeptide comprises a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of LFA-3 or a fragment thereof fused to the Fc domain of an immunoglobulin molecule or a fragment thereof. In another embodiment, a fusion protein that immunospecifically binds to a CD2 polypeptide comprises a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of LFA-3 or a fragment thereof fused to the CH2 and/or CH3 region of the Fc domain of an immunoglobulin molecule. In another embodiment, a fusion protein that immunospecifically binds to a CD2 polypeptide comprises a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of LFA-3 or a fragment thereof fused to the CH2, CH3, and hinge regions of the Fc domain of an immunoglobulin molecule.

In another embodiment, a fusion protein that immunospecifically binds to a CD2 polypeptide comprises a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of an extracellular domain of LFA-3 (e.g., amino acid residues 1 to 187 of SEQ ID NO:7) fused to the Fe domain of an immunoglobulin molecule or a fragment thereof. In another embodiment, a fusion protein that immunospecifically binds to a CD2 polypeptide comprise a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of an extracellular domain of LFA-3 (e.g., amino acid residues 1 to 187 of SEQ ID NO:7) fused to the CH2 and/or CH3 region of the Fe domain of an immunoglobulin molecule. In another embodiment, a fusion protein that immunospecifically binds to a CD2 polypeptide comprise a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of an extracellular domain of LFA-3 (e.g., amino acid residues 1 to 187 of SEQ ID NO:7) fused to the CH2, CH3, and hinge regions of the Fe domain of an immunoglobulin molecule.

In another embodiment, a fusion protein that immunospecifically binds to a CD2 polypeptide comprises a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of a fragment of an extracellular domain of LFA-3 (e.g., amino acid residues 1 to 92, amino acid residues 1 to 85, amino acid residues 1 to 80, amino acid residues 1 to 75, amino acid residues 1 to 70, amino acid residues 1 to 65, or amino acid residues 1 to 60 SEQ ID NO:7) fused to the Fc domain of an immunoglobulin molecule or a fragment thereof.

In another embodiment, a fusion protein that immunospecifically binds to a CD2 polypeptide comprises a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of a fragment of an extracellular domain of LFA-3 (e.g., amino acid residues 1 to 92, amino acid residues 1 to 85, amino acid residues 1 to 80, amino acid residues 1 to 75, amino acid residues 1 to 70, amino acid residues 1 to 65, or amino acid residues 1 to 60 SEQ ID NO:7) fused to the CH2 and/or CH3 region of the Fc domain of an immunoglobulin molecule.

In another embodiment, a fusion protein that immunospecifically binds to a CD2 polypeptide comprises a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of a fragment of an extracellular domain of LFA-3 (e.g., amino acid residues 1 to 92, amino acid residues 1 to 85, amino acid residues 1 to 80, amino acid residues 1 to 75, amino acid residues 1 to 70, amino acid residues 1 to 65, or amino acid residues 1 to 60 SEQ ID NO:7) fused to the CH2, CH3, and hinge regions of the Fc domain of an immunoglobulin molecule.

The present invention provides fusion proteins that immunospecifically bind to a CD2 polypeptide comprising the Fc domain of an immunoglobulin molecule or a fragment thereof fused to a polypeptide encoded by a nucleic acid molecule that hybridizes to the nucleotide sequence encoding LFA-3 or a fragment thereof.

In a specific embodiment, a fusion protein that immunospecifically binds to a CD2 polypeptide comprises the Fe domain of an immunoglobulin molecule or a fragment thereof fused to a polypeptide encoded by a nucleic acid molecule that hybridizes to the nucleotide sequence encoding LFA-3 or a fragment thereof under stringent conditions, e.g., hybridization to filter-bound DNA in 6× sodium chloride/sodium citrate (SSC) at about 45° C. followed by one or more washes in 0.2× SSC/0.1% SDS at about 50-65° C., under highly stringent conditions, e.g., hybridization to filter-bound nucleic acid in 6× SSC at about 45° C. followed by one or more washes in 0.1× SSC/0.2% SDS at about 68° C., or under other stringent hybridization conditions which are known to those of skill in the art (see, for example, Ausubel, F. M. et al., eds., 1989, Current Protocols in Molecular Biology, Vol. 1, Green Publishing Associates, Inc. and John Wiley & Sons, Inc., New York at pages 6.3.1-6.3.6 and 2.10.3).

In another embodiment, a fusion protein that immunospecifically binds to a CD2 polypeptide comprises the Fe domain of an immunoglobulin molecule or a fragment thereof fused to a polypeptide encoded by a nucleic acid molecule that hybridizes to the nucleotide sequence encoding an extracellular domain of LFA-3 (e.g., amino acid residues 1 to 187 of SEQ ID NO:7) under stringent conditions, e.g., hybridization to filter-bound DNA in 6× sodium chloride/sodium citrate (SSC) at about 45° C. followed by one or more washes in 0.2× SSC/0.1% SDS at about 50-65° C., under highly stringent conditions, e.g., hybridization to filter-bound nucleic acid in 6× SSC at about 45° C. followed by one or more washes in 0.1× SSC/0.2% SDS at about 68° C., or under other stringent hybridization conditions which are known to those of skill in the art (see, for example, Ausubel, F. M. et al., eds., 1989, Current Protocols in Molecular Biology, Vol. 1, Green Publishing Associates, Inc. and John Wiley & Sons, Inc., New York at pages 6.3.1-6.3.6 and 2.10.3).

In yet another embodiment, a fusion protein that immunospecifically binds to a CD2 polypeptide comprises the Fe domain of an immunoglobulin molecule or a fragment thereof fused to a polypeptide encoded by a nucleic acid molecule that hybridizes to the nucleotide sequence encoding the amino acid sequence of a fragment of an extracellular domain of LFA-3 (e.g., amino acid residues 1 to 92, amino acid residues 1 to 85, amino acid residues 1 to 80, amino acid residues 1 to 75, amino acid residues 1 to 70, amino acid residues 1 to 65, or amino acid residues 1 to 60 SEQ ID NO:7) under stringent conditions, e.g., hybridization to filter-bound DNA in 6× sodium chloride/sodium citrate (SSC) at about 45° C. followed by one or more washes in 0.2× SSC/0.1% SDS at about 50-65° C., under highly stringent conditions, e.g., hybridization to filter-bound nucleic acid in 6× SSC at about 45° C. followed by one or more washes in 0.1× SSC/0.2% SDS at about 68° C., or under other stringent hybridization conditions which are known to those of skill in the art (see, for example, Ausubel, F. M. et al., eds., 1989, Current Protocols in Molecular Biology, Vol. 1, Green Publishing Associates, Inc. and John Wiley & Sons, Inc., New York at pages 6.3.1-6.3.6 and 2.10.3).

4.2.3.1. Fusion Protein Conjugates

The present invention also encompasses fusion proteins that immunospecifically bind to a CD2 polypeptide fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag.

The present invention further encompasses fusion proteins that immunospecifically bind to a CD2 polypeptide conjugated to a therapeutic agent. A fusion protein that immunospecifically binds to a CD2 polypeptide may be conjugated to a therapeutic moiety such as a cytotoxin, e.g., a cytostatic or cytocidal agent, an agent which has a potential therapeutic benefit, or a radioactive metal ion, e.g., alpha-emitters. A cytotoxin or cytotoxic agent includes any agent that is detrimental to cells. Examples of a cytotoxin or cytotoxic agent include, but are not limited to, paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Agents which have a potential therapeutic benefit include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Further, a fusion protein that immunospecifically binds to a CD2 polypeptide may be conjugated to a therapeutic agent or drug moiety that modifies a given biological response. Agents which have a potential therapeutic benefit or drug moieties are not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, IFN-α, IFN-β, NGF, PDGF, TPA, an apoptotic agent, e.g., TNF-α, TNF-β, AIM I (see, International Publication No. WO 97/33899), AIM II (see, International Publication No. WO 97/34911), Fas Ligand (Takahashi et al., 1994, J. Immunol., 6:1567-1574), and VEGF (see, International Publication No. WO 99/23105), a thrombotic agent or an anti-angiogenic agent, e.g., angiostatin or endostatin; or, a biological response modifier such as, for example, a lymphokine (e.g., IL-1, IL-2, IL-6, IL-10, GM-CSF, and G-CSF), or a growth factor (e.g., GH).

4.3. Prophylactic and Therapeutic Uses of CD2 Antagonists

The present invention is directed to therapies which involve administering CD2 antagonists, particularly CD2 binding molecules, to a subject, preferably a human subject, for preventing, treating, or ameliorating an autoimmune disorder or an inflammatory disorder or one or more symptoms thereof. In particular, the present invention is directed to therapies which involve administering CD2 antagonists, particularly CD2 binding molecules, to a subject, preferably a human subject, for preventing, treating, or ameliorating one or more symptoms of psoriasis.

Examples of autoimmune disorders include, but are not limited to, but not limited to, alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Ménière's disease, mixed connective tissue disease, multiple sclerosis, type 1 or immune-mediated diabetes mellitus, myasthenia gravis, pemphigus vulgaris, pernicious anemia, polyarteritis nodosa, polychrondritis, polyglandular syndromes, polymyalgia rheumatica, polymyositis and dermatomyositis, primary agammaglobulinemia, primary biliary cirrhosis, psoriasis, psoriatic arthritis, Raynauld's phenomenon, Reiter's syndrome, Rheumatoid arthritis, sarcoidosis, scleroderma, Sjögren's syndrome, stiff-man syndrome, systemic lupus erythematosus, lupus erythematosus, takayasu arteritis, temporal arteristis/giant cell arteritis, ulcerative colitis, uveitis, vasculitides such as dermatitis herpetiformis vasculitis, vitiligo, and Wegener's granulomatosis. Examples of inflammatory disorders include, but are not limited to, asthma, encephilitis, inflammatory bowel disease, chronic obstructive pulmonary disease (COPD), inflammatory osteolysis, allergic disorders, septic shock, pulmonary fibrosis, undifferentitated spondyloarthropathy, undifferentiated arthropathy, arthritis, inflammatory osteolysis, and chronic inflammation resulting from chronic viral or bacteria infections. Examples of the types of psoriasis which can be treated in accordance with the compositions and methods of the invention include, but are not limited to, plaque psoriasis, pustular psoriasis, erythrodermic psoriasis, guttate psoriasis and inverse psoriasis.

The compositions and methods of the invention are particularly useful for the prevention, treatment or amelioration of autoimmune disorders characterized by increased T cell infiltration of lymphocytes into affected dermal or epidermal tissues, or autoimmune disorders characterized by increased T cell activation and/or abnormal antigen presentation. The compositions and methods are also useful for the prevention, treatment or amelioration of inflammatory disorders characterized by increased T cell activation and/or abnormal antigen presentation. Further, compositions and methods can be applied to skin conditions characterized by increased T cell activation and/or abnormal T cell activation such as, e.g., psoriasis, ultraviolet damage, atopic dermatitis, cutaneous T cell lymphoma, allergic and irritant contact dermatitis, lichen planus, alopecia areata, pyoderma gangrenosum, vitiligo, ocular, cicatricial pemphigoid, lupus erythematous, scleroderma, and urticaria.

In one embodiment, one or more pharmaceutical compositions comprising one or more CD2 antagonists are not administered to an immunocompromised or immunosuppressed mammal (e.g., an HIV patient) to prevent, treat or ameliorate one or more symptoms of autoimmune disorder or inflammatory disorder. In another embodiment, a first dose of a pharmaceutical composition comprising one or more CD2 antagonists (e.g., one or more CD2 binding molecules) is not administered to a subject with a mean absolute lymphocyte count under 750 cells/mm³, 800 cells/mm³, 850 cells/mm³, 900 cells/mm³, 950 cells/mm³, 1000 cells/mm³, 1050 cells/mm³, 1100 cells/mm³, 1200 cells/mm³ or 1250 cells/mm³ to prevent, treat or ameliorate an autoimmune disorder or an inflammatory disorder or one or more symptoms thereof. In another embodiment, one or more pharmaceutical compositions comprising one or more CD2 antagonists (e.g., one or more CD2 binding molecules) are administered to a subject to prevent, treat or ameliorate psoriasis or one or more symptoms thereof that is refractory to topical or steroid treatment. In another embodiment, one or more pharmaceutical compositions comprising one or more CD2 antagonists (e.g., one or more CD2 binding molecules) are administered to a subject that has not been treated with an immunosuppressant agent to prevent, treat or ameliorate psoriasis or one or more symptoms thereof. In an alternative embodiment, one or more pharmaceutical compositions comprising one or more CD2 antagonists (e.g., one or more CD2 binding molecules) are administered to a subject who has been treated or who is being treated with another immunosuppressant agent to prevent, treat or ameliorate psoriasis or one or more symptoms thereof.

In another embodiment, one or more pharmaceutical compositions comprising one or more CD2 antagonists (e.g., one or more CD2 binding molecules) are administered to prevent, treat or ameliorate one or more symptoms of severe psoriasis in a subject. In another embodiment, one or more pharmaceutical compositions comprising one or more CD2 antagonists (e.g., one or more CD2 binding molecules) are administered to prevent, treat or ameliorate one or more symptoms of moderate psoriasis in a subject. In yet another embodiment, one or more pharmaceutical compositions comprising one or more CD2 antagonists (e.g., one or more CD2 binding molecules) are administered to prevent, treat or one or more symptoms of less than moderate psoriasis in a subject. In accordance with these embodiments, the severity of psoriasis is determined by the Psoriasis Activity and Severity Index (PASI) score and/or by the physician's global assessment. See, e.g., Frederiksson et al., 1978, Dermatologica 157:238-244, Harai et al., 2000, Int. J. Dermatol. 39(12):913-918, Devrimci-Ozguven et al., 2000, J. Eur. Acad. Dermatol. Venereol. 14(4):267-71, Jemec et al., 1997, Acta Derm. Venereol. 77(5):392-393, Husted et al., 1995, Clin. Exp. Rheumatol. 13(4):439-43 for information regarding PASI scoring and other types of scoring utilized to measure the severity of psoriasis and to determine any changes in a subject's psoriasis condition.

In a one embodiment, a subject is administered one or more doses of 150 μg/kg or less, preferably 125 μg/kg or less, 100 μg/kg or less, 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.5 μg/kg or less of one or more CD2 antagonists to prevent, treat or ameliorate one or more symptoms of an autoimmune disorder or an inflammatory disorder. In a specific embodiment, a subject is administered one or more doses of 150 μg/kg or less, preferably 125 μg/kg or less, 100 μg/kg or less, 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.5 μg/kg or less of one or more CD2 binding molecules to prevent, treat or ameliorate one or more symptoms of an autoimmune disorder or an inflammatory disorder.

In a preferred embodiment, a subject is administered one or more doses of a 200 μg/kg or less, preferably 175 μg/kg or less, 150 μg/kg or less, 125 μg/kg or less, 100 μg/kg or less, 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.5 μg/kg or less of one or more CD2 antagonists to prevent, treat or ameliorate one or more symptoms of psoriasis. In another preferred embodiment, a subject is administered one or more doses of a 200 μg/kg or less, preferably 175 μg/kg or less, 150 μg/kg or less, 125 μg/kg or less, 100 μg/kg or less, 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.5 μg/kg or less of one or more CD2 binding molecules to prevent, treat or ameliorate one or more symptoms of psoriasis.

In a specific embodiment, a subject is intramuscularly administered one or more doses of a 200 μg/kg or less, preferably 175 μg/kg or less, 150 μg/kg or less, 125 μg/kg or less, 100 μg/kg or less, 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.5 μg/kg or less of one or more CD2 binding molecules to prevent, treat or ameliorate one or more symptoms of an autoimmune disorder or an inflammatory disorder. In another embodiment, a subject is subcutaneously administered one or more doses of a 200 μg/kg or less, preferably 175 μg/kg or less, 150 μg/kg or less, 125 μg/kg or less, 100 μg/kg or less, 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.5 μg/kg or less of one or more CD2 binding molecules to prevent, treat or ameliorate one or more symptoms of an autoimmune disorder or an inflammatory disorder. In another embodiment, a subject is intravenously administered one or more doses of a 100 μg/kg or less, preferably 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.5 μg/kg or less of one or more CD2 binding molecules to prevent, treat or ameliorate one or more symptoms of an autoimmune disorder or an inflammatory disorder.

In a one embodiment, a subject is administered one or more unit doses of 0.25 mg to 20 mg, 0.25 to 15 mg, 0.25 to 12 mg, 0.25 to 10 mg, 0.25 to 8 mg, 1 mg to 20 mg, 1 mg to 15 mg, 1 mg to 12 mg, 1 mg to 10 mg, or 1 mg to 8 mg of one or more CD2 antagonists to prevent, treat or ameliorate one or more symptoms of an autoimmune disorder or an inflammatory disorder. In a specific embodiment, a subject is administered one or more unit doses of 0.25 mg to 20 mg, 0.25 to 15 mg, 0.25 to 12 mg, 0.25 to 10 mg, 0.25 to 8 mg, 1 mg to 20 mg, 1 mg to 15 mg, 1 mg to 12 mg, 1 mg to 10 mg, or 1 mg to 8 mg of one or more CD2 binding molecules to prevent, treat or ameliorate one or more symptoms of an autoimmune disorder or an inflammatory disorder. In another embodiment, a subject is administered one or more unit doses of 0.5 mg, 1 mg, 1.5 mg, 2 mg, 3 mg, 4 mg, 5 mg, 6 mg, 7 mg, 8 mg, 9 mg, 10 mg, 11 mg, 12 mg, 13 mg, 14 mg, 15 mg, or 16 mg of one or more CD2 binding molecules to prevent, treat or ameliorate one or more symptoms of an autoimmune disorder or an inflammatory disorder.

In another embodiment, a subject is administered one or more doses of a prophylactically or therapeutically effective amount of one or more CD2 antagonists, wherein the prophylactically or therapeutically effective amount is not the same for each dose. In another embodiment, a subject is administered one or more doses of a prophylactically or therapeutically effective amount of one or more CD2 binding molecules, wherein the prophylactically or therapeutically effective amount is not the same for each dose.

In another embodiment, a subject is administered one or more doses of a prophylactically or therapeutically effective amount of one or more CD2 antagonists, wherein the dose of a prophylactically or therapeutically effective amount said CD2 antagonists administered to said subject is increased by, e.g., 0.01 μg/kg, 0.02 μg/kg, 0.04 μg/kg, 0.05 μg/kg, 0.06 μg/kg, 0.08 μg/kg, 0.1 μg/kg, 0.2 μg/kg, 0.25 μg/kg, 0.5 μg/kg, 0.75 μg/kg, 1 μg/kg, 1.5 μg/kg, 2 μg/kg, 4 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65%g/kg, 70 μg/kg, 75 μg/kg, 80 μg/kg, 85 μg/kg, 90 μg/kg, 95 μg/kg, 100 μg/kg, or 125 μg/kg, as treatment progresses. In another embodiment, a subject is administered one or more doses of a prophylactically or therapeutically effective amount of one or more CD2 binding molecules, wherein the dose of a prophylactically or therapeutically effective amount said CD2 binding molecules administered to said subject is increased by, e.g., 0.01 μg/kg, 0.02 μg/kg, 0.04 μg/kg, 0.05 μg/kg, 0.06 μg/kg, 0.08 μg/kg, 0.1 μg/kg, 0.2 μg/kg, 0.25 μg/kg, 0.5 μg/kg, 0.75 μg/kg, 1 μg/kg, 1.5 μg/kg, 2 μg/kg, 4 μg/kg, 5 μg/kg, 10 μg/kg, 15 μg/kg, 20 μg/kg, 25 μg/kg, 30 pig/kg, 35 μg/kg, 40 μg/kg, 45 μg/kg, 50 pig/kg, 55 μg/kg, 60 μg/kg, 65 μg/kg, 70 μg/kg, 75 μg/kg, 80 pig/kg, 85 μg/kg, 90 μg/kg, 95 μg/kg, 100 μg/kg, or 125 μg/kg, as treatment progresses.

In another embodiment, a subject is administered one or more doses of a prophylactically or therapeutically effective amount of one or more CD2 antagonists, wherein the dose of a prophylactically or therapeutically effective amount of said CD2 antagonists administered to said subject is decreased by, e.g., 0.01 pig/kg, 0.02 μg/kg, 0.04 μg/kg, 0.05 μg/kg, 0.06 μg/kg, 0.08 μg/kg, 0.1 μg/kg, 0.2 μg/kg, 0.25 μg/kg, 0.5 μg/kg, 0.75 μg/kg, 1 μg/kg, 1.5 μg/kg, 2 μg/kg, 4 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, or 125 μg/kg, as treatment progresses. In another embodiment, a subject is administered one or more doses of a prophylactically or therapeutically effective amount of one or more CD2 binding molecules, wherein the dose of a prophylactically or therapeutically effective amount of said CD2 binding molecules administered to said subject is decreased by, e.g., 0.01 μg/kg, 0.02 μg/kg, 0.04 μg/kg, 0.05 μg/kg, 0.06 μg/kg, 0.08 μg/kg, 0.1 μg/kg, 0.2 μg/kg, 0.25 μg/kg, 0.5 μg/kg, 0.75 μg/kg, 1 μg/kg, 1.5 μg/kg, 2 μg/kg, 4 μg/kg, 5 μg/kg, 10 pig/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, or 125 μg/kg, as treatment progresses.

In a specific embodiment, a subject is administered a first dose of a prophylactically or therapeutically effective amount of one or more CD2 binding molecules to prevent, treat or ameliorate one or more symptoms of an autoimmune disorder or an inflammatory disorder, wherein said administration of the first dose results in CD2 binding molecules binding to at least 20%, preferably at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 98% of the CD2 polypeptides expressed by peripheral blood lymphocytes (preferably, peripheral blood T-cells) for at least 30 minutes, preferably at least 1 hour, 2 hours, at least 4 hours, 5 hours, at least 10 hours, at least 12 hours, at least 24 hours, at least 36 hours, at least 48 hours, at least 72 hours, or at least 1 week after the administration of the first dose and prior to the administration of a subsequent dose. Preferably, the effective amount of said CD2 binding molecules is a dose of 150 μg/kg or less, preferably 125 μg/kg or less, 100 μg/kg or less, 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 pig/kg or less, 1 μg/kg or less, 0.5 pig/kg or less, or 0.5 μg/kg or less.

In a specific embodiment, a subject is administered a first dose and one or more subsequent doses of a prophylactically or therapeutically effective amount of one or more CD2 binding molecules to prevent, treat or ameliorate one or more symptoms of an autoimmune disorder or an inflammatory disorder, wherein a subsequent dose is only administered when less than 55%, less than 50%, less than 45%, less than 40%, less than 35%, less than 30%, less than 35%, less than 30%, less than 25%, less than 20%, less than 15%, less than 10%, or less than 5% of the CD2 polypeptides expressed by peripheral blood lymphocytes (preferably, peripheral blood T-cells) are bound by CD2 binding molecules. In a preferred embodiment, a subject is administered a first dose and one or more subsequent doses of a prophylactically or therapeutically effective amount of one or more CD2 binding molecules to prevent, treat or ameliorate one or more symptoms of psoriasis, wherein a subsequent dose is only administered when less than 55%, less than 50%, less than 45%, less than 40%, less than 35%, less than 30%, less than 35%, less than 30%, less than 25%, less than 20%, less than 15%, less than 10%, or less than 5% of the CD2 polypeptides expressed by peripheral blood lymphocytes (preferably, peripheral blood T-cells) are bound by CD2 binding molecules.

In another embodiment, a subject is administered a first dose and one or more subsequent doses of a prophylactically or therapeutically effective amount of one or more CD2 binding molecules to prevent, treat or ameliorate one or more symptoms of an autoimmune disorder or an inflammatory disorder, wherein a subsequent dose is only administered when less than 55%, less than 50%, less than 45%, less than 40%, less than 35%, less than 30%, less than 35%, less than 30%, less than 25%, less than 20%, less than 15%, less than 10%, or less than 5% of the CD2 polypeptides expressed by CD4+T-cells are bound by CD2 binding molecules. In another embodiment, a subject is administered a first dose and one or more subsequent doses of a prophylactically or therapeutically effective amount of one or more CD2 binding molecules to prevent, treat or ameliorate one or more symptoms of an autoimmune disorder or an inflammatory disorder, wherein a subsequent dose is only administered when less than 55%, less than 50%, less than 45%, less than 40%, less than 35%, less than 30%, less than 35%, less than 30%, less than 25%, less than 20%, less than 15%, less than 10%, or less than 5% of the CD2 polypeptides expressed by CD8⁺ T-cells are bound by CD2 binding molecules. In yet another embodiment, a subject is administered a first dose and one or more subsequent doses of a prophylactically or therapeutically effective amount of one or more CD2 binding molecules to prevent, treat or ameliorate one or more symptoms of an autoimmune disorder or an inflammatory disorder, wherein a subsequent dose is only administered when less than 55%, less than 50%, less than 45%, less than 40%, less than 35%, less than 30%, less than 35%, less than 30%, less than 25%, less than 20%, less than 15%, less than 10%, or less than 5% of the CD2 polypeptides expressed by memory T-cells (i.e., CD45RO⁺ T-cells) are bound by CD2 binding molecules.

In a specific embodiment, the percentage of CD2 polypeptides expressed by peripheral blood lymphocytes (preferably, peripheral blood T-cells) bound by CD2 binding molecules is assessed before or after or both before and after the administration of one or more doses of a prophylactically or therapeutically effective amount of one or more CD2 binding molecules to a subject to determine whether one or more subsequent doses of a prophylactically or therapeutically effective amount of one or more CD2 binding molecules should be administered to said subject. A subsequent dose of a prophylactically or therapeutically effective amount of one or more CD2 binding molecules may or may not be administered to said subject if the percentage of CD2 polypeptides expressed by peripheral blood lymphocytes (preferably, peripheral blood T-cells) bound by CD2 binding molecules is 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, or 98% or more. However, a subsequent dose of a prophylactically or therapeutically effective amount of one or more CD2 binding molecules is administered to said subject if the percentage of CD2 polypeptides expressed by peripheral blood lymphocytes (preferably, peripheral blood T-cells) bound by CD2 binding molecules is less than 25%, less than 20%, less than 15%, less than 10%, or less than 5%.

In another embodiment, a subject is administered a first dose of a prophylactically or therapeutically effective amount of one or more CD2 binding molecules, and after the administration of the first dose but prior to the administration of one or more subsequent doses of said CD2 binding molecules, the percentage of CD2 polypeptides expressed by peripheral blood lymphocytes (preferably, T-cells) bound by CD2 binding molecules is assessed. In accordance with this embodiment, one or more subsequent doses of said CD2 binding molecules may be administered if the percentage of CD2 polypeptides bound by CD2 binding molecules is less than 55%, less than 50%, less than 45%, less than 40%, less than 35%, less than 30%, less than 35%, less than 30%, less than 25%. However, one or more subsequent doses of said CD2 binding molecules is administered if the percentage of CD2 polypeptides bound by CD2 binding molecules is less than 20%, less than 15%, less than 10%, or less than 5%.

In another embodiment, the percentage of CD2 polypeptides expressed by peripheral blood lymphocytes (preferably, T-cells) bound by CD2 binding molecules is assessed prior to the administration of a first, second, third, fourth, fifth, sixth, seventh, eighth, ninth, tenth, eleventh, twelfth, thirteenth, fourteenth, or fifteenth dose of a prophylactically or therapeutically effective amount of one or more CD2 binding molecules to a subject with an autoimmune or inflammatory disorder. In another embodiment, the percentage of CD2 polypeptides expressed by peripheral blood lymphocytes (preferably, T-cells) bound by CD2 binding molecules is assessed prior to the administration of a second, fourth, sixth, eighth, tenth, twelfth, and/or fourteenth dose of a prophylactically or therapeutically effective amount of one or more CD2 binding molecules to a subject with an autoimmune or inflammatory disorder. In another embodiment, the percentage of CD2 polypeptides expressed by peripheral blood lymphocytes (preferably, T-cells) bound by CD2 binding molecules is assessed prior to the administration of every second, every third, or every fourth dose of a prophylactically or therapeutically effective amount of one or more CD2 binding molecules to a subject with an autoimmune or inflammatory disorder.

In a specific embodiment, the mean absolute lymphocyte count in a subject with an autoimmune or inflammatory is assessed before and/or after the administration of one or more doses of a prophylactically or therapeutically effective amount of one or more CD2 antagonists to determine whether one or more subsequent doses of a prophylactically or therapeutically effective amount of one or more CD2 antagonists should be administered to said subject. In another embodiment, the mean absolute lymphocyte count in a subject with an autoimmune or inflammatory is assessed before and/or after the administration of one or more doses of a prophylactically or therapeutically effective amount of one or more CD2 binding molecules to determine whether one or more subsequent doses of a prophylactically or therapeutically effective amount of one or more CD2 binding molecules should be administered to said subject. Preferably, a subsequent dose of a prophylactically or therapeutically effective amount of one or more CD2 binding molecules is not administered to said subject if the lymphocyte count is less than 800 cells/mm³, less than 750 cells/mm³, less than 700 cells/mm³, less than 650 cells/mm³, less than 600 cells/mm³ or 500 cells/mm³ or less.

In another embodiment, the mean absolute lymphocyte count in a subject with an autoimmune disorder or an inflammatory disorder is determined prior to the administration of a first dose of a prophylactically or therapeutically effective amount of one or more CD2 antagonists and the mean absolute lymphocyte count is monitored prior to the administration of one or more subsequent doses of a prophylactically or therapeutically effective amount of one or more CD2 antagonists. Preferably, the mean absolute lymphocyte count in the subject is at least 900 cells/mm³, preferably at least 950 cells/mm³, at least 1000 cells/mm³, at least 1050 cells/mm³, at least 100 cells/mm³, at least 1200 cells/mm³, or at least 1250 cells/ml prior to the administration of a first dose of one or more CD2 binding molecules.

In another embodiment, the mean absolute lymphocyte count in a subject with an autoimmune disorder or an inflammatory disorder is determined prior to the administration of a first dose of a prophylactically or therapeutically effective amount of one or more CD2 binding molecules and the mean absolute lymphocyte count is monitored prior to the administration of one or more subsequent doses of a prophylactically or therapeutically effective amount of one or more CD2 binding molecules. Preferably, the mean absolute lymphocyte count in the subject is at least 900 cells/mm³, preferably at least 950 cells/mm³, at least 1000 cells/mm³, at least 1050 cells/mm³, at least 1100 cells/mm³, at least 1200 cells/mm³, or at least 1250 cells/mm³ prior to the administration of a first dose of one or more CD2 binding molecules.

In another embodiment, the mean absolute lymphocyte count in a subject with an autoimmune disorder or an inflammatory disorder is determined prior to the administration of a first dose of a prophylactically or therapeutically effective amount of one or more CD2 antagonists and the mean absolute lymphocyte count is monitored prior to the administration of each subsequent dose of a prophylactically or therapeutically effective amount of one or more CD2 binding molecules. In accordance with this embodiment, a subsequent dose is not be administered to said mammal if the lymphocyte count is less than 800 cells/mm³, less than 750 cells/mm³, less than 700 cells/mm³, less than 650 cells/mm³, less than 600 cells/mm³ or 500 cells/mm³ or less. Preferably, at least one of the CD2 antagonists is a CD2 binding molecule.

In another embodiment, the mean absolute lymphocyte count in a subject with an autoimmune disorder or inflammatory disorder is determined prior to the administration of a first, second, third, fourth, fifth, sixth, seventh, eighth, ninth, tenth, eleventh, twelfth, thirteenth, fourteenth, or fifteenth dose of a prophylactically or therapeutically effective amount of one or more CD2 antagonists (preferably, one or more CD2 binding molecules). In another embodiment, the mean absolute lymphocyte count in a subject with an autoimmune disorder or inflammatory disorder is determined prior to the administration of a second, fourth, sixth, eighth, tenth, twelfth, and/or fourteenth dose of a prophylactically or therapeutically effective amount of one or more CD2 antagonists (preferably, one or more CD2 binding molecules). In another embodiment, the mean absolute lymphocyte count in a subject with an autoimmune disorder or an inflammatory disorder is determined prior to the administration of every second, every third, or every fourth dose of a prophylactically or therapeutically effective amount of one or more CD2 antagonists (preferably, one or more CD2 binding molecules).

In another embodiment, a mean absolute lymphocyte count of approximately 700 cells/ml to approximately 1200 cells/ml, approximately 700 cells/ml to approximately 100 cells/ml, approximately 700 cells/ml to approximately 1000 cells/ml, approximately 700 to approximately 900 cells/ml, approximately 750 cells/ml to approximately 1200 cells/ml, approximately 750 cells/ml to approximately 1100 cells/ml, approximately 750 cells/ml to approximately 1000 cells/ml, approximately 750 cells/ml to approximately 900 cells/ml, approximately 800 cells/ml to approximately 1200 cells/ml, approximately 800 cells/ml to approximately 1100 cells/ml, approximately 800 cells/ml to approximately 1000 cells/ml, approximately 900 cells/ml to approximately 1200 cells/ml, approximately 900 cells/ml to approximately 1100 cells/ml, approximately 900 cells/ml to approximately 1000 cells/ml, or approximately 1000 cells to approximately 1200 cells/ml is maintained in a subject with an autoimmune disorder or inflammatory disorder by administering one or more doses of a prophylactically or therapeutically effective amount of one or more CD2 binding molecules. In another embodiment, a mean absolute lymphocyte count of approximately 700 cells/ml to below 1000 cells/ml is maintained in a subject with an autoimmune disorder or an inflammatory disorder by administering one or more doses of a prophylactically or therapeutically effective amount of one or more CD2 binding molecules.

In another embodiment, the mean absolute lymphocyte count in a subject with an autoimmune disorder or inflammatory disorder is determined prior to the administration of a first dose of a prophylactically or therapeutically effective amount of one or more CD2 binding molecules and the lymphocyte count is assessed after the administration of said first dose to determine if another dose of a prophylactically or therapeutically effective amount of one or more CD2 binding molecules is necessary. In accordance with this embodiment, a subject is administered another dose if the mean absolute lymphocyte count is above 1000 cells/mm³ or if the lymphocyte count is only less than 1%, less than 2%, less than 5%, less than 10%, or less than 15% lower than the lymphocyte count in said subject prior to the administration a the first dose.

In another embodiment, a reduction of between 20% to 40%, 25% to 40%, 30% to 40%, 35% to 40%, 20% to 30%, or 25% to 30% in mean absolute lymphocyte count of is maintained in a subject with an autoimmune disorder or an inflammatory disorder by administering one or more doses of a prophylactically or therapeutically effective amount of one or more CD2 antagonists. In another embodiment, a reduction of between 20% to 40%, 25% to 40%, 30% to 40%, 35% to 40%, 20% to 30%, or 25% to 30% in mean absolute lymphocyte count of is maintained in a subject with an autoimmune disorder or an inflammatory disorder by administering one or more doses of a prophylactically or therapeutically effective amount of one or more CD2 binding molecules.

In another embodiment, a human is administered doses of a prophylactically or therapeutically effective amount of one or more CD2 binding molecules to achieve a reduction in said human's PASI score by at least 20%, at least 35%, at least 30%, at least 40%, at least 45%, at least 50%, at least 55%, at least 60%, at least 65%, at least 70%, at least 75%, at least 80%, or at least 85%. In another embodiment, a human is administered doses of a prophylactically or therapeutically effective amount of one or more CD2 binding molecules to achieve an improvement in said human's global assessment score by at least 25%, at least 35%, at least 30%, at least 40%, at least 45%, at least 50%, at least 55%, at least 60%, at least 65%, at least 70%, at least 75%, at least 80%, at least 85%, at least 90%, or at least 95%. In yet another embodiment, a human is administered doses of a prophylactically or therapeutically effective amount of one or more CD2 binding molecules to achieve a reduction in said human's PASI score by at least 20%, at least 35%, at least 30%, at least 40%, at least 45%, at least 50%, at least 55%, at least 60%, at least 65%, at least 70%, at least 75%, at least 80%, or at least 85% and an improvement in said human's global assessment score by at least 25%, at least 35%, at least 30%, at least 40%, at least 45%, at least 50%, at least 55%, at least 60%, at least 65%, at least 70%, at least 75%, at least 80%, at least 85%, at least 90%, or at least 95%.

In a specific embodiment a subject is administered a dose of a prophylactically or therapeutically effective amount of one or more CD2 binding molecules, wherein said dose achieves a serum level of CD2 binding molecules of 0.5 ng/ml to 100 ng/ml. In a preferred embodiment a subject is administered a dose of a prophylactically or therapeutically effective amount of MEDI-507, a derivative, analog or antigen binding fragment thereof, wherein said dose achieves a serum level of CD2 binding molecules of 0.5 ng/ml to 100 ng/ml. Preferably, such serum levels are achieved and/or maintained at least 30 minutes, 1 hour, 2 hours, 4 hours, 6 hours, 8 hours, 10 hours, 12 hours, 24 hours, 48 hours, 72 hours, or 1 week.

In another embodiment, a human is administered doses of a prophylactically or therapeutically effective amount of one or more CD2 binding molecules to prevent, treat or ameliorate one or more symptoms of a psoriasis, said doses being effective to achieve a reduction in said human's PASI score by at least 20%, at least 35%, at least 30%, at least 40%, at least 45%, at least 50%, at least 55%, at least 60%, at least 65%, at least 70%, at least 75%, at least 80%, or at least 85% but insufficient to cause a reduction in lymphocyte count to below 900 cells/mm³, 850 cells/mm³, 800 cells/mm³, 750 cells/mm³, 700 cells/mm³, 650 cells/mm³, 600 cells/mm³, 550 cells/mm³, or 500 cells/mm³. In another embodiment, a human is administered doses of a prophylactically or therapeutically effective amount of one or more CD2 binding molecules to prevent, treat or ameliorate one or more symptoms of a psoriasis, said doses being effective to achieve an improvement in said human's global assessment score by at least 25%, at least 35%, at least 30%, at least 40%, at least 45%, at least 50%, at least 55%, at least 60%, at least 65%, at least 70%, at least 75%, at least 80%, at least 85%, at least 90%, or at least 95%.

In a specific embodiment, the administration of one or more doses of a prophylactically or therapeutically effective amount of one or more CD2 antagonists does not induce or reduces relative to other immunosuppressive agents one or more of the following unwanted or adverse effects: vital sign abnormalities (fever, tachycardia, bardycardia, hypertension, hypotension), hematological events (anemia, lymphopenia, leukopenia, thrombocytopenia), headache, chills, dizziness, nausea, asthenia, back pain, chest pain (chest pressure), diarrhea, myalgia, pain, pruritus, psoriasis, rhinitis, sweating, injection site reaction, vasodilatation, an increased risk of opp\portunistic infection, and an increased risk of developing certain types of cancer. In another specific embodiment, the administration of one or more doses of a prophylactically or therapeutically effective amount of one or more CD2 binding molecules does not induce or reduces relative to other immunosuppressive agents one or more of the following unwanted or adverse effects: vital sign abnormalities (fever, tachycardia, bardycardia, hypertension, hypotension), hematological events (anemia, lymphopenia, leukopenia, thrombocytopenia), headache, chills, dizziness, nausea, asthenia, back pain, chest pain (chest pressure), diarrhea, myalgia, pain, pruritus, psoriasis, rhinitis, sweating, injection site reaction, vasodilatation, an increased risk of opportunistic infection, and an increased risk of developing certain types of cancer.

4.4. Methods of Administering of CD2 Antagonists

The present invention provides compositions for the treatment, prophylaxis, and amelioration of one or more symptoms associated with an autoimmune or inflammatory disorder. In a specific embodiment, a composition comprises one or more CD2 antagonists. In another embodiment, a composition comprises one or more nucleic acid molecules encoding one or more CD2 antagonists. In another embodiment, a composition comprises one or more CD2 binding molecules. In another embodiment, a composition comprises one or more nucleic acid molecules encoding one or more CD2 binding molecules.

In a specific embodiment, a composition comprises a CD2 binding molecule, wherein said CD2 binding molecule is a fusion protein that immunospecifically binds to a CD2 polypeptide. In a preferred embodiment, a composition comprises a CD2 binding molecule, wherein said CD2 binding molecule is an antibody that immunospecifically bind to a CD2 polypeptide. In another preferred embodiment, a composition comprises a CD2 binding molecule, wherein said CD2 binding molecule is a human or humanized monoclonal antibody. In yet another preferred embodiment, a composition comprises MEDI-507, a analog, derivative, fragment thereof that immunospecifically binds to CD2 polypeptides.

In a preferred embodiment, a composition of the invention is a pharmaceutical composition. Such compositions comprise a prophylactically or therapeutically effective amount of one or more CD2 antagonists, and a pharmaceutically acceptable carrier. Preferably, such compositions comprise a prophylactically or therapeutically effective amount of one or more CD2 binding molecules, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a prophylactically or therapeutically effective amount of a prophylactic or therapeutic agent preferably in purified form, together with a suitable amount of carrier so as to provide the form for proper administration to the patient. The formulation should suit the mode of administration. In a preferred embodiment, the pharmaceutical compositions are sterile and in suitable form for administration to a subject, preferably an animal subject, more preferably a mammalian subject, and most preferably a human subject.

In a specific embodiment, it may be desirable to administer the pharmaceutical compositions of the invention locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers. Preferably, when administering a CD2 binding molecule, care must be taken to use materials to which the CD2 binding molecule does not absorb.

In another embodiment, the composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In yet another embodiment, the composition can be delivered in a controlled release or sustained release system. In one embodiment, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of the antibodies of the invention or fragments thereof (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. No. 5,679,377; U.S. Pat. No. 5,916,597; U.S. Pat. No. 5,912,015; U.S. Pat. No. 5,989,463; U.S. Pat. No. 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a sustained release formulation is inert, free of leachable impurities, stable on storage, sterile, and biodegradable. In yet another embodiment, a controlled or sustained release system can be placed in proximity of the therapeutic target, i.e., the lungs, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising one or more antibodies of the invention or fragments thereof. See, e.g., U.S. Pat. No. 4,526,938, .PCT publication WO 91/05548, PCT publication WO 96/20698,.Ning et al., 1996, “Intratumoral Radioimmunotheraphy of a Human Colon Cancer Xenograft Using a Sustained-Release Gel,” Radiotherapy & Oncology 39:179-189,.Song et al, 1995, “Antibody Mediated Lung Targeting of Long-Circulating Emulsions,” PDA Journal of Pharmaceutical Science & Technology 50:372-397, Cleek et al., 1997, “Biodegradable Polymeric Carriers for a bFGF Antibody for Cardiovascular Application,” Pro. Int'l. Symp. Control. Rel. Bioact. Mater. 24:853-854, and Lam et al., 1997, “Microencapsulation of Recombinant Humanized Monoclonal Antibody for Local Delivery,” Proc. Int'l. Symp. Control Rel. Bioact. Mater. 24:759-760, each of which is incorporated herein by reference in their entirety.

In a specific embodiment where the composition of the invention is a nucleic acid encoding a CD2 binding molecule, the nucleic acid can be administered in vivo to promote expression of its encoded CD2 binding molecule, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression by homologous recombination.

A pharmaceutical composition of the invention is formulated to be compatible with its intended route of administration. Examples of routes of administration include, but are not limited to, parenteral, e.g., intravenous, intradermal, subcutaneous, oral (e.g., inhalation), intranasal, transdermal (topical), transmucosal, and rectal administration. In a specific embodiment, the composition is formulated in accordance with routine procedures as a pharmaceutical composition adapted for intravenous, subcutaneous, intramuscular, oral, intranasal or topical administration to human beings. In a preferred embodiment, a pharmaceutical composition is formulated in accordance with routine procedures for subcutaneous administration to human beings. Typically, compositions for intravenous administration are solutions in sterile isotonic aqueous buffer. Where necessary, the composition may also include a solubilizing agent and a local anesthetic such as lignocamne to ease pain at the site of the injection.

If the compositions of the invention are to be administered topically, the compositions can be formulated in the form of, e.g., an ointment, cream, transdermal patch, lotion, gel, shampoo, spray, aerosol, solution, emulsion, or other form well-known to one of skill in the art. See, e.g., Remington's Pharmaceutical Sciences and Introduction to Pharmaceutical Dosage Forms, 4th ed., Lea & Febiger, Philadelphia, Pa. (1985). For non-sprayable topical dosage forms, viscous to semi-solid or solid forms comprising a carrier or one or more excipients compatible with topical application and having a dynamic viscosity preferably greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, preferably in combination with a solid or liquid inert carrier, is packaged in a mixture with a pressurized volatile (e.g., a gaseous propellant, such as freon), or in a squeeze bottle. Moisturizers or humectants can also be added to pharmaceutical compositions and dosage forms if desired. Examples of such additional ingredients are well-known in the art.

If the compositions of the invention are to be administered intranasally, the compositions can be formulated in an aerosol form, spray, mist or in the form of drops. In particular, prophylactic or therapeutic agents for use according to the present invention can be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

If the compositions of the invention are to be administered orally, the compositions can be formulated orally in the form of, e.g., tablets, capsules, cachets, gelcaps, solutions, suspensions and the like. Tablets or capsules can be prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well-known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated for slow release, controlled release or sustained release of a prophylactic or therapeutic agent(s).

The compositions of the invention may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compositions of the invention may also be formulated in rectal compositions such as suppositories or retention enemas, e.g., containing conventional suppository bases such as cocoa butter or other glycerides.

In addition to the formulations described previously, the compositions of the invention may also be formulated as a depot preparation. Such long acting formulations may be administered by implantation (for example subcutaneously or intramuscularly) or by intramuscular injection. Thus, for example, the compositions may be formulated with suitable polymeric or hydrophobic materials (for example as an emulsion in an acceptable oil) or ion exchange resins, or as sparingly soluble derivatives, for example, as a sparingly soluble salt.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In particular, the invention provides that one or more CD2 antagonists, or pharmaceutical compositions of the invention is packaged in a hermetically sealed container such as an ampoule or sachette indicating the quantity of the agent. In one embodiment, one or more of the CD2 antagonists, or pharmaceutical compositions of the invention is supplied as a dry sterilized lyophilized powder or water free concentrate in a hermetically sealed container and can be reconstituted, e.g., with water or saline to the appropriate concentration for administration to a subject. Preferably, one or more of the CD2 antagonists, or pharmaceutical compositions of the invention is supplied as a dry sterile lyophilized powder in a hermetically sealed container at a unit dosage of at least 5 mg, more preferably at least 10 mg, at least 15 mg, at least 25 mg, at least 35 mg, at least 45 mg, at least 50 mg, at least 75 mg, or at least 100 mg. The lyophilized prophylactic or therapeutic agents, or pharmaceutical compositions of the invention should be stored at between 2 and 8° C. in its original container and the prophylactic or therapeutic agents, or pharmaceutical compositions of the invention should be administered within 1 week, preferably within 5 days, within 72 hours, within 48 hours, within 24 hours, within 12 hours, within 6 hours, within 5 hours, within 3 hours, or within 1 hour after being reconstituted. In an alternative embodiment, one or more of the CD2 antagonists, or pharmaceutical compositions of the invention is supplied in liquid form in a hermetically sealed container indicating the quantity and concentration of the agent. Preferably, the liquid form of the administered composition is supplied in a hermetically sealed container at least 0.25 mg/ml, more preferably at least 0.5 mg/ml, at least 1 mg/ml, at least 2.5 mg/ml, at least 5 mg/ml, at least 8 mg/ml, at least 10 mg/ml, at least 15 mg/kg, at least 25 mg/ml, at least 50 mg/ml, at least 75 mg/ml or at least 100 mg/ml. The liquid form should be stored at between 2° C. and 8° C. in its original container.

In a preferred embodiment, the invention provides that MEDI-507 is packaged in a hermetically sealed container such as an ampoule or sachette indicating the quantity of MEDI-507. In one embodiment, MEDI-507 is supplied as a dry sterilized lyophilized powder or water free concentrate in a hermetically sealed container and can be reconstituted, e.g., with water or saline to the appropriate concentration for administration to a subject. Preferably, MEDI-507 is supplied as a dry sterile lyophilized powder in a hermetically sealed container at a unit dosage of at least 5 mg, more preferably at least 10 mg, at least 15 mg, at least 25 mg, at least 35 mg, at least 45 mg, at least 50 mg, at least 75 mg, or at least 100 mg. In an alternative embodiment, MEDI-507 is supplied in liquid form in a hermetically sealed container indicating the quantity and concentration of the MEDI-507. Preferably, the liquid form of MEDI-507 is supplied in a hermetically sealed container at least 0.25 mg/ml, more preferably at least 0.5 mg/ml, at least 1 mg/ml, at least 2.5 mg/ml, at least 5 mg/ml, at least 8 mg/ml, at least 10 mg/ml, at least 15 mg/kg, at least 25 mg/ml, at least 50 mg/ml, at least 75 mg/ml or at least 100 mg/ml.

The compositions may, if desired, be presented in a pack or dispenser device that may contain one or more unit dosage forms containing the active ingredient. The pack may for example comprise metal or plastic foil, such as a blister pack.

Generally, the ingredients of the compositions of the invention are derived from a subject that is the same species origin or species reactivity as recipient of such compositions. Thus, in a preferred embodiment, human or humanized antibodies are administered to a human patient for therapy or prophylaxis.

The amount of the composition of the invention which will be effective in the treatment, prevention or amelioration of one or more symptoms associated with an inflammatory disease or autoimmune disorder can be determined by standard clinical techniques. The precise dose to be employed in the formulation will also depend on the route of administration, and the seriousness of the condition, and should be decided according to the judgment of the practitioner and each patient's circumstances. Effective doses may be extrapolated from dose-response curves derived from in vitro or animal model test systems.

For antibodies, proteins, polypeptides, peptides and fusion proteins encompassed by the invention, the dosage administered to a patient is typically 0.0001 mg/kg to 100 mg/kg of the patient's body weight. Preferably, the dosage administered to a patient is between 0.0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg to 0.25 mg/kg, 0.0001 to 0.15 mg/kg, 0.0001 to 0.10 mg/kg, 0.001 to 0.5 mg/kg, 0.01 to 0.25 mg/kg or 0.01 to 0.10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention or fragments thereof may be reduced by enhancing uptake and tissue penetration of the antibodies by modifications such as, for example, lipidation.

In a specific embodiment, the dosage of a composition of the invention or a CD2 antagonist administered to prevent, treat or ameliorate one or more symptoms associated with an autoimmune or inflammatory disorder in a patient is 200 μg/kg or less, preferably 150 μg/kg or less, 125 μg/kg or less, 100 μg/kg or less, 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.5 μg/kg or less of a patient's body weight. In another embodiment, the dosage of a composition of the invention or a CD2 antagonist is a unit dose of 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7 mg, 0.25 mg to 5 mg, 0.5 mg to 2.5 mg, 1 mg to 20 mg, 1 mg to 1 mg, 1 mg to 12 mg, 1 mg to 10 mg, 1 mg to 8 mg, 1 mg to 7 mg, 1 mg to 5 mg, or 1 mg to 2.5 mg.

In other embodiments, a subject is administered one or more doses of 200 μg/kg or less, 150 μg/kg or less, preferably 125 μg/kg or less, 100 μg/kg or less, 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.4 μg/kg or less of MEDI-507 to prevent, treat or ameliorate one or more symptoms associated with an autoimmune disorder or inflammatory disorder. Preferably, such doses are administered intravaneously to a subject with an autoimmune disorder or an inflammatory disorder. In a preferred embodiment, a subject is administered one or more doses of 60 μg/kg or less, preferably 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.4 μg/kg or less of MEDI-507 to prevent, treat or ameliorate one or more symptoms associated with psoriasis.

In a specific embodiment, a subject is administered one or more unit doses of 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 mg to 2.5 mg, 0.25 mg to 20 mg, 0.25 to 15 mg, 0.25 to 12 mg, 0.25 to 10 mg, 0.25 to 8 mg, 0.25 mg to 7 mg, 0.25 mg to 5 mg, 0.25 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg of MEDI-507 to prevent, treat or ameliorate one or more symptoms associated with an autoimmune disorder or inflammatory disorder. In another embodiment, a subject is administered one or more unit doses of 0.1 mg, 0.25 mg, 0.5 mg, 1 mg, 1.5 mg, 2 mg, 3 mg, 4 mg, 5 mg, 6 mg, 7 mg, 8 mg, 9 mg, 10 mg, 11 mg, 12 mg, 13 mg, 14 mg, 15 mg, or 16 mg of MEDI-507 to prevent, treat or ameliorate one or more symptoms associated with an autoimmune disorder or inflammatory disorder. Preferably, the unit doses of MEDI-507 are administered subcutaneously to a subject with an autoimmune or inflammatory disorder. In a preferred embodiment, a subject is administered one or more unit doses of 10 mg, 9 mg, 8 mg, 7 mg, 6 mg, 5 mg, 4 mg, 3 mg, 2 mg or 1 mg to prevent, treat or ameliorate one or more symptoms associated with psoriasis.

In a specific embodiment, a subject is administered one or more doses of a prophylactically or therapeutically effective amount of MEDI-507, wherein the prophylactically or therapeutically effective amount is not the same for each dose. In another embodiment, a subject, preferably a human, is administered one or more doses of a prophylactically or therapeutically effective amount of MEDI-507, wherein the dose of a prophylactically or therapeutically effective amount MEDI-507 administered to said subject is increased by, e.g., 0.01 μg/kg, 0.02 μg/kg, 0.04 μg/kg, 0.05 μg/kg, 0.06 μg/kg, 0.08 μg/kg, 0.1 μg/kg, 0.2 μg/kg, 0.25 μg/kg, 0.5 μg/kg, 0.75 μg/kg, 1 μg/kg, 1.5 μg/kg, 2 μg/kg, 4 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, or 125 μg/kg, as treatment progresses. In another embodiment, a subject, preferably a human, is administered one or more doses of a prophylactically or therapeutically effective amount of MEDI-507, wherein the dose of a prophylactically or therapeutically effective amount of MEDI-507 administered to said subject is decreased by, e.g., 0.01 μg/kg, 0.02 μg/kg, 0.04 μg/kg, 0.05 μg/kg, 0.06 μg/kg, 0.08 μg/kg, 0.1 μg/kg, 0.2 μg/kg, 0.25 μg/kg, 0.5 μg/kg, 0.75 μg/kg, 1 μg/kg, 1.5 μg/kg, 2 μg/kg, 4 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, or 125 μg/kg, as treatment progresses.

In yet another embodiment, a subject is administered a dose of a prophylactically or therapeutically effective amount of one or more CD2 antagonists, wherein administration of the dose to said subject achieves a mean absolute lymphocyte count of approximately 500 cells/mm³ to below 1500 cells/mm³, preferably below 1400 cells/mm³, below 1300 cells/mm³, below 1250 cells/mm³, below 1200 cells/mm³, below 1100 cells/mm³ or below 1000 cell/mm³. In another embodiment, a subject is administered a dose of a prophylactically or therapeutically effective amount of one of more CD2 binding molecule, wherein administration of the dose to said subject achieves a a mean absolute lymphocyte count of approximately 500 cells/mm³ to below 1500 cells/mm³, preferably below 1400 cells/mm³, below 1300 cells/mm³, below 1250 cells/mm³, below 1200 cells/mm³, below 1100 cells/mm³ or below 1000 cell/mm³. In a preferred embodiment, a subject is administered a dose of a prophylactically or therapeutically effective amount of MEDI-507, wherein administration of the dose of MEDI-507 to said subject achieves in said subject a mean absolute lymphocyte count of approximately 500 cells/mm³ to below 1500 cells/mm³, preferably below 1400 cells/mm³, below 1300 cells/mm³, below 1250 cells/mm³, below 1200 cells/mm³, below 1100 cells/mm³ or below 1000 cell/mm³.

In another embodiment, a subject is administered a dose of a prophylactically or therapeutically effective amount of one or more CD2 antagonists, wherein administration of the dose to said subject results in at least a 10%, preferably 15%, 20%, 25%, 30%, 35%, 40%, 45%, 50%, 55% or 60% reduction in mean absolute lymphocyte count. In another embodiment, a subject is administered a dose of a prophylactically or therapeutically effective amount of one of more CD2 binding molecule, wherein administration of the dose to said subject results in at least a 10%, preferably 15%, 20%, 25%, 30%, 35%, 40%, 45%, 50%, 55% or 60% reduction in mean absolute lymphocyte count. In a preferred embodiment, a subject is administered a dose of a prophylactically or therapeutically effective amount of MEDI-507, wherein administration of the dose of MEDI-507 to said subject results in at least a 10%, preferably 15%, 20%, 25%, 30%, 35%, 40%, 45%, 50%, 55% or 60% reduction in mean absolute lymphocyte count.

In other embodiments, a subject is administered one or more doses of a prophylactically or therapeutically effective amount of one or more CD2 binding molecules, wherein the dose of a prophylactically or therapeutically effective amount of said CD2 binding molecules administered achieves at least 20% to 25%, 25% to 30%, 30% to 35%, 35% to 40%, 40% to 45%, 45% to 50%, 50% to 55%, 55% to 60%, 60% to 65%, 65% to 70%, 70% to 75%, 75% to 80%, up to at least 80% of CD2 polypeptide being bound by CD2 binding molecules. In yet other embodiments, a subject is administered one or more doses of a prophylactically or therapeutically effective amount of MEDI-507, wherein the dose of a prophylactically or therapeutically effective amount of MEDI-507 administered achieves at least 20% to 25%, 25% to 30%, 30% to 35%, 35% to 40%, 40% to 45%, 45% to 50%, 50% to 55%, 55% to 60%, 60% to 65%, 65% to 70%, 70% to 75%, 75% to 80%, up to at least 80% of CD2 polypeptide being bound by CD2 binding molecules.

One or more CD2 antagonists may be advantageously utilized in combination with one or more currently used, previously used or known therapeutic or prophylactic agents for a particular autoimmune disorder or inflammatory disorder. In particular, one or more CD2 binding molecules may be advantageously utilized in combination with anti-angiogenic agents (e.g., angiostatin, an antagonist of Integrin αᵥβ₃ (e.g., VITAXIN™), or with a TNFα antagonist (e.g., anti-TNFα antibody), or endostatin), or with cytokine inhibitors, which, for example, serve to reduce adverse side effects associated with the administration of one or more CD2 binding molecules. One or more CD2 binding molecules may also be advantageously utilized in combination with immunosuppressive agents (e.g., Cyclosporin A (CsA), methylprednisolone (MP), corticosteroids, OKT3 (anti-CD3 monoclonal human antibody), mycophenolate mofetil, rapamycin (sirolimus), mizoribine, deoxyspergualin, macrolide antibiotics (e.g., FK506 (tacrolimus), brequinar, and malononitriloamindes.(e.g., leflunamide)), and anti-IL-2R antibodies (e.g., anti-Tac monoclonal antibody and BT 536)), or with lymphokines or hematopoietic growth factors (e.g. IL-10), or with anti-angiogenic factors (e.g., angiostatin, an antagonist of Integrin αᵥβ₃ (e.g., VITAXIN™), a TNFα antagonist (e.g., anti-TNFα antibody), or endostatin) for the prevention, treatment or amelioration of one or more symptoms associated with an autoimmune disorder or an inflammatory disorder.

One or more CD2 binding molecules may be utilized in combination with one or more corticosteroid and/or one or more nonsteroidal anti-inflammatory agents to prevent, treat, or ameliorate one or more symptoms of systemic lupus erythematosus. In another example, one or more CD2 binding molecules may be utilized in combination with aspirin, leflunomide (Arava), one or more non-steroidal anti-inflammatory agents (e.g., ibuprofen, fenoprofen, indomethacin, and naproxen), one or more Cox-2 inhibitors (e.g., rofecoxib (Vioxx) and celecoxib (Celebrex)), and/or one or more anti-TNFα agents (e.g., infliximab (Remicade) and etanercept (Enbrel)) to prevent, treat or ameliorate one or more symptoms of rheumatoid arthritis.

In preferred embodiment, one or more CD2 binding molecules are utilized in combination with one or more known therapeutic or prophylactic agents for psoriasis. Examples of known treatments for psoriasis include, but are not limited to, hydoxyurea, methotrexate, cyclosporin, acitretin, ultraviolet B radiation phototherapy, photochemotherapy, topical corticosteriods (e.g., diflorasone diacetate, clobetasol propionate, halobetasol propionate, betamethasone dipopionate, fluocinonide, halcinonide, desoximetasone, triamcinolone acetonide, fluticasone propionate, flucinolone acetonide, flurandrenolide, mometasone furoate, betamethasone, fluticasone propionate, flucinolong acetonide, aclometasome dipropionate, desonide, and hydrocortisone), topical vitamin D3 analogs (e.g., calcipotriene), dithranol (anthralin), coal tar, salicyclic acid, topical retinoids (e.g., tazarotene), macrolide antibiotics (e.g., tacrolimus), anti-CD3 monoclonal antibodies, anti-CD4 monoclonal antibodies, anti-CD11a monoclonal antibodies, anti-IL-2Rα monoclonal antibodies, anti-ICAM 1 antibodies, anti-LFA1 antibodies, anti-CD80 monoclonal antibodies, CTLA4Ig, and emollients. For reviews of treatments for psoriasis see, e.g., Ashcroft et al., 2000, Journal of Clinical Pharmacy and Therapeutics 25: 1-10; Karasek, 1999, Cutis 64:319-322; Drew, Primary Care 27:385-406; Lebwohl, 2000, Dermatologic Clinics 18:13-19; and Peters et al., 2000, Am. J. Health-Sys. Pharm. 57:645-659.

4.4.1. Gene Therapy

In a specific embodiment, nucleic acids comprising sequences encoding CD2 antagonists, are administered to treat, prevent or ameliorate one or more symptoms of an autoimmune disorder, in particular psoriasis, by way of gene therapy. Gene therapy refers to therapy performed by the administration to a subject of an expressed or expressible nucleic acid. In this embodiment of the invention, the nucleic acids produce their encoded CD2 antagonist (preferably, CD2 binding molecule) that mediates a prophylactic or therapeutic effect.

Any of the methods for gene therapy available in the art can be used according to the present invention. Exemplary methods are described below.

For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, Science 260:926-932 (1993); and Morgan and Anderson, 1993, Ann. Rev. Biochem. 62:191-217; May, 1993, TIBTECH 1 (5):155-215. Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a preferred aspect, a composition of the invention comprises nucleic acids encoding a CD2 binding molecule, said nucleic acids being part of an expression vector that expresses the CD2 binding molecule in a suitable host. In particular, such nucleic acids have promoters, preferably heterologous promoters, operably linked to the antibody coding region, said promoter being inducible or constitutive, and, optionally, tissue-specific. In another particular embodiment, nucleic acid molecules are used in which the CD2 binding molecule coding sequences and any other desired sequences are flanked by regions that promote homologous recombination at a desired site in the genome, thus providing for intrachromosomal expression of the antibody encoding nucleic acids (Koller and Smithies, 1989, Proc. Natl. Acad. Sci. USA 86:8932-8935; Zijlstra et al., 1989, Nature 342:435-438). In specific embodiments, the expressed CD2 binding molecule is an antibody. In a preferred embodiment, the expressed CD2 binding molecule is LoCD2a/BTI-322 or MEDI-507. In other embodiments, the expressed CD2 binding molecule is a fusion protein.

Delivery of the nucleic acids into a subject may be either direct, in which case the subject is directly exposed to the nucleic acid or nucleic acid-carrying vectors, or indirect, in which case, cells are first transformed with the nucleic acids in vitro, then transplanted into the subject. These two approaches are known, respectively, as in vivo or ex vivo gene therapy.

In a specific embodiment, the nucleic acid sequences are directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, 1987, J. Biol. Chem. 262:4429-4432) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180; WO 92/22635; WO92/203 16; WO93/14188, WO 93/20221). Alternatively, the nucleic acid can be introduced intracellularly and incorporated within host cell DNA for expression, by homologous recombination (Koller and Smithies, 1989, Proc. Natl. Acad. Sci. USA 86:8932-8935; and Zijlstra et al., 1989, Nature 342:435-438).

In a specific embodiment, viral vectors that contains nucleic acid sequences encoding a CD2 binding molecule are used. For example, a retroviral vector can be used (see Miller et al., 1993, Meth. Enzymol. 217:581-599). These retroviral vectors contain the components necessary for the correct packaging of the viral genome and integration into the host cell DNA. The nucleic acid sequences encoding the antibody to be used in gene therapy are cloned into one or more vectors, which facilitates delivery of the gene into a subject. More detail about retroviral vectors can be found in Boesen et al., 1994, Biotherapy 6:291-302, which describes the use of a retroviral vector to deliver the mdr 1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Klein et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; Mastrangeli et al., 1993, J. Clin. Invest. 91:225-234; PCT Publication WO94/12649; and Wang et al., 1995, Gene Therapy 2:775-783. In a preferred embodiment, adenovirus vectors are used.

Adeno-associated virus (AAV) has also been proposed for use in gene therapy (Walsh et al., 1993, Proc. Soc. Exp. Biol. Med. 204:289-300; and U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcellmediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; Clin. Pharma. Ther. 29:69-92 (1985)) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a subject by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a preferred embodiment, the cell used for gene therapy is autologous to the subject.

In an embodiment in which recombinant cells are used in gene therapy, nucleic acid sequences encoding a CD2 binding molecule are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention (see e.g., PCT Publication WO 94/08598; Stemple and Anderson, 1992, Cell 7 1:973-985; Rheinwald, 1980, Meth. Cell Bio. 21A:229; and Pittelkow and Scott, 1986, Mayo Clinic Proc. 61:771).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

4.5. CD2 Antagonists Characterization and Demonstration of Therapeutic or Prophylactic Utility

CD2 binding molecules may be characterized in a variety of ways. In particular, CD2 binding molecules may be assayed for the ability to immunospecifically bind to a CD2 polypeptide. Such an assay may be performed in solution (e.g., Houghten, 1992, Bio/Techniques 13:412-421), on beads (Lam, 1991, Nature 354:82-84), on chips (Fodor, 1993, Nature 364:555-556), on bacteria (U.S. Pat. No. 5,223,409), on spores (U.S. Pat. Nos. 5,571,698; 5,403,484; and 5,223,409), on plasmids (Cull et al., 1992, Proc. Natl. Acad. Sci. USA 89:1865-1869) or on phage (Scott and Smith, 1990, Science 249:386-390; Devlin, 1990, Science 249:404-406; Cwirla et al., 1990, Proc. Natl. Acad. Sci. USA 87:6378-6382; and Felici, 1991, J. Mol. Biol. 222:301-310) (each of these references is incorporated herein in its entirety by reference). CD2 binding molecules that have been identified to immunospecifically bind to a CD2 polypeptide can then be assayed for their specificity and affinity for a CD2 polypeptide.

CD2 binding molecules may be assayed for immunospecific binding to a CD2 polypeptide and cross-reactivity with other polypeptides by any method known in the art. Immunoassays which can be used to analyze immunospecific binding and cross-reactivity include, but are not limited to, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the CD2 binding molecule of interest to the cell lysate, incubating for a period of time (e.g., 1 to 4 hours) at 40° C., adding protein A and/or protein G sepharose beads to the cell lysate, incubating for about an hour or more at 40° C., washing the beads in lysis buffer and resuspending the beads in SDS/sample buffer. The ability of the CD2 binding molecule of interest to immunoprecipitate a particular antigen can be assessed by, e.g., western blot analysis. One of skill in the art would be knowledgeable as to the parameters that can be modified to increase the binding of the CD2 binding molecule to a CD2 polypeptide and decrease the background (e.g., pre-clearing the cell lysate with sepharose beads). For further discussion regarding immunoprecipitation protocols see, e.g., Ausubel et al, eds, 1994, Current Protocols in Molecular Biology, Vol. 1, John Wiley & Sons, Inc., New York at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with CD2 binding molecule of interest (e.g., an antibody of interest) diluted in blocking buffer, washing the membrane in washing buffer, blocking the membrane with an antibody (which recognizes the CD2 binding molecule) conjugated to an enzymatic substrate (e.g., horseradish peroxidase or alkaline phosphatase) or radioactive molecule (e.g., ³²P or ¹²⁵I) diluted in blocking buffer, washing the membrane in wash buffer, and detecting the presence of the CD2 polypeptide. One of skill in the art would be knowledgeable as to the parameters that can be modified to increase the signal detected and to reduce the background noise. For further discussion regarding western blot protocols see, e.g., Ausubel et al, eds, 1994, Current Protocols in Molecular Biology, Vol. 1, John Wiley & Sons, Inc., New York at 10.8.1.

ELISAs comprise preparing CD2 polypeptide, coating the well of a 96 well microtiter plate with the CD2 polypeptide, adding the CD2 binding molecule of interest conjugated to a detectable compound such as an enzymatic substrate (e.g., horseradish peroxidase or alkaline phosphatase) to the well and incubating for a period of time, and detecting the presence of the CD2 polypeptide. In ELISAs the CD2 binding molecule of interest does not have to be conjugated to a detectable compound; instead, an antibody (which recognizes the CD2 binding molecule of interest) conjugated to a detectable compound may be added to the well. Further, instead of coating the well with the CD2 polypeptide, the CD2 binding molecule may be coated to the well. In this case, an antibody conjugated to a detectable compound may be added following the addition of the CD2 polypeptide to the coated well. One of skill in the art would be knowledgeable as to the parameters that can be modified to increase the signal detected as well as other variations of ELISAs known in the art. For further discussion regarding ELISAs see, e.g., Ausubel et al, eds, 1994, Current Protocols in Molecular Biology, Vol. 1, John Wiley & Sons, Inc., New York at 11.2.1.

The binding affinity of a CD2 binding molecule to a CD2 polypeptide and the off-rate of an CD2 binding molecule-CD2 polypeptide interaction can be determined by competitive binding assays. One example of a competitive binding assay is a radioimmunoassay comprising the incubation of labeled CD2 polypeptide (e.g., ³H or ¹²⁵I) with the CD2 binding molecule of interest in the presence of increasing amounts of unlabeled CD2 polypeptide, and the detection of the CD2 binding molecule bound to the labeled CD2 polypeptide. The affinity of a CD2 binding molecule for a CD2 polypeptide and the binding off-rates can be determined from the data by scatchard plot analysis. Competition with a second CD2 binding molecule can also be determined using radioimmunoassays. In this case, a CD2 polypeptide is incubated with a CD2 binding molecule conjugated to a labeled compound (e.g., ³H or ¹²⁵I) in the presence of increasing amounts of a second unlabeled CD2 binding molecule.

In a preferred embodiment, BIAcore kinetic analysis is used to determine the binding on and off rates of CD2 binding molecules to a CD2 polypeptide. BIAcore kinetic analysis comprises analyzing the binding and dissociation of a CD2 polypeptide from chips with immobilized CD2 binding molecules on their surface.

The CD2 antagonists, in particular CD2 binding molecules, can also be assayed for their ability to inhibit the binding of a CD2 polypeptide to LFA-3 using techniques known to those of skill in the art. For example, cells expressing LFA-3 can be contacted with a CD2 polypeptide in the presence or absence of CD2 binding molecule and the ability of the CD2 binding molecule to inhibit LFA-3's binding can measured by, for example, flow cytometry or a scintillation assay. The CD2 polypeptide or the CD2 binding molecule can be labeled with a detectable compound such as a radioactive label (e.g., ³²P, ³⁵S, and 125I) or a fluorescent label (e.g., fluorescein isothiocyanate, rhodamine, phycoerythrin, phycocyanin, allophycocyanin, o-phthaldehyde and fluorescamine) to enable detection of an interaction between the CD2 polypeptide and the CD2 binding molecule. Alternatively, the ability of CD2 binding molecules to inhibit CD2 polypeptide from binding to LFA-3 can be determined in cell-free assays. For example, CD2 polypeptide can be contacted with a CD2 binding molecule and the ability of the CD2 binding molecule to inhibit CD2 polypeptide from binding to LFA-3 can be determined. Preferably, the CD2 binding molecule is immobilized on a solid support and the CD2 polypeptide is labeled with a detectable compound. Alternatively, the CD2 polypeptide is immobilized on a solid support and the CD2 binding molecule is labeled with a detectable compound. The CD2 polypeptide may be partially or completely purified (e.g., partially or completely free of other polypeptides) or part of a cell lysate.

The CD2 antagonists, in particular CD2 binding molecules, and compositions of the invention can also be assayed for their ability to modulate T-cell activation. T-cell activation can be determined by measuring, e.g., changes in the level of expression of cytokines and/or T-cell activation markers. Techniques known to those of skill in the art, including, but not limited to, immunoprecipitation followed by western blot analysis, ELISAs, flow cytometry, Northern blot analysis, and RT-PCR can be used to measure the expression cytokines and T-cell activation markers. In a preferred embodiment, a CD2 binding molecule or composition of the invention is tested for its ability to induce the expression of IFN-γand/or IL-2.

The CD2 antagonists, in particular CD2 binding molecules, and compositions of the invention can also be assayed for their ability to induce T-cell signaling. The ability of a CD2 binding molecule or a composition of the invention induce T-cell signaling can be assayed, e.g., by kinase assays and electrophoretic shift assays (EMSAs).

CD2 antagonists, in particular CD2 binding molecules, and compositions of the invention can be tested in vitro or in vivo for their ability to modulate T-cell proliferation. For example, the ability of a CD2 binding molecule or a composition of the invention to modulate T-cell proliferation can be assessed by, e.g., ³H-thymidine incorporation, trypan blue cell counts, and fluorescence activated cell sorting (FACS).

CD2 antagonists, in particular CD2 binding molecules, and compositions of the invention can be tested in vitro or in vivo for their ability to induce cytolysis. For example, the ability of a CD2 binding molecule or a composition of the invention to induce cytolysis can be assessed by, e.g., ⁵¹Cr-release assays.

CD2 antagonists, in particular CD2 binding molecules, and compositions of the invention can be tested in vitro or in vivo for their ability to induce cytolysis. For example, the ability of a CD2 binding molecule or a composition of the invention to induce cytolysis can be assessed by, e.g., ⁵¹Cr-release assays.

CD2 antagonists, in particular CD2 binding molecules, and compositions of the invention can be tested in vitro or in vivo for their ability to mediate the depletion of peripheral blood T-cell and/or the depletion of NK cells. For example, the ability of a CD2 binding molecule or a composition of the invention to mediate the depletion of peripheral blood T-cell can be assessed by, e.g., measuring T-cell counts using flow cytometry analysis.

CD2 antagonists, in particular CD2 binding molecules, and compositions of the invention can be tested in vivo for their ability to mediate peripheral blood lymphocyte counts. For example, the ability of a CD2 binding molecule or a composition of the invention to mediate peripheral blood lymphocyte counts can be assessed by, e.g., obtaining a sample of peripheral blood from a subject, separating the lymphocytes from other components of peripheral blood such as plasma using, e.g., a Ficoll gradient, and counting the lymphocytes using trypan blue.

Several aspects of the pharmaceutical compositions or CD2 antagonists of the invention are preferably tested in vitro, in a cell culture system, and in an animal model organism, such as a rodent animal model system, for the desired therapeutic activity prior to use in humans. For example, assays which can be used to determine whether administration of a specific pharmaceutical composition is indicated, include cell culture assays in which a patient tissue sample is grown in culture, and exposed to or otherwise contacted with a pharmaceutical composition, and the effect of such composition upon the tissue sample is observed. The tissue sample can be obtained by biopsy from the patient. This test allows the identification of the therapeutically most effective tumor-targeted bacteria and the therapeutically most effective therapeutic molecule(s) for each individual patient. In various specific embodiments, in vitro assays can be carried out with representative cells of cell types involved in an autoimmune or inflammatory disorder (e.g., T cells), to determine if a pharmaceutical composition of the invention has a desired effect upon such cell types.

In accordance with the invention, clinical trials with human subjects need not be performed in order to demonstrate the prophylactic and/or therapeutic efficacy of CD2 antagonists, in particular CD2 binding molecules. In vitro and animal model studies using CD2 antagonists can be extrapolated to humans and are sufficient for demonstrating the prophylactic and/or therapeutic utility of said CD2 antagonists.

CD2 antagonists can be tested in suitable animal model systems prior to use in humans. Such animal model systems include, but are not limited to, rats, mice, chicken, cows, monkeys, pigs, dogs, rabbits, etc. Any animal system well-known in the art may be used. In a specific embodiment of the invention, CD2 antagonists are tested in a mouse model system. Such model systems are widely used and well-known to the skilled artisan. CD2 antagonists can be administered repeatedly. Several aspects of the procedure may vary. Said aspects include the temporal regime of administering CD2 antagonists, and whether such agents are administered separately or as an admixture.

The anti-inflammatory activity of CD2 antagonists or pharmaceutical compositions of invention can be determined by using various experimental animal models of inflammatory arthritis known in the art and described in Crofford L. J. and Wilder R. L., “Arthritis and Autoimmunity in Animals”, in Arthritis and Allied Conditions: A Textbook of Rheumatology, McCarty et al.(eds.), Chapter 30 (Lee and Febiger, 1993). Experimental and spontaneous animal models of inflammatory arthritis and autoimmune rheumatic diseases can also be used to assess the anti-inflammatory activity of CD2 antagonists or pharmaceutical compositions of invention. The following are some assays provided as examples and not by limitation.

The principle animal models for arthritis or inflammatory disease known in the art and widely used include: adjuvant-induced arthritis rat models, collagen-induced arthritis rat and mouse models and antigen-induced arthritis rat, rabbit and hamster models, all described in Crofford L. J. and Wilder R. L., “Arthritis and Autoimmunity in Animals”, in Arthritis and Allied Conditions: A Textbook of Rheumatology, McCarty et al.(eds.), Chapter 30 (Lee and Febiger, 1993), incorporated herein by reference in its entirety. A collagen-induced arthritis (CIA) is an animal model for the human autoimmune disease rheumatoid arthritis (RA) (Trenthom et al., 1977, J. Exp. Med. 146:857). This disease can be induced in many species by the administration of heterologous type II collagen (Courtenay et al., 1980, Nature 283:665; and Cathcart et at, 1986, Lab. Invest. 54:26). With respect to animal models of arthritis see, in addition, e.g., Holmdahl, R., 1999, Curr. Biol. 15:R528-530.

The anti-inflammatory activity of CD2 antagonists or pharmaceutical compositions of invention can be assessed using a carrageenan-induced arthritis rat model. Carrageenan-induced arthritis has also been used in rabbit, dog and pig in studies of chronic arthritis or inflammation. Quantitative histomorphometric assessment is used to determine therapeutic efficacy. The methods for using such a carrageenan-induced arthritis model is described in Hansra P. et al., “Carrageenan-Induced Arthritis in the Rat,” Inflammation, 24(2): 141-155, (2000). Also commonly used are zymosan-induced inflammation animal models as known and described in the art.

The anti-inflammatory activity of CD2 antagonists or pharmaceutical compositions of invention can also be assessed by measuring the inhibition of carrageenan-induced paw edema in the rat, using a modification of the method described in Winter C. A. et al., “Carrageenan-Induced Edema in Hind Paw of the Rat as an Assay for Anti-inflammatory Drugs” Proc. Soc. Exp. Biol Med. 111, 544-547, (1962). This assay has been used as a primary in vivo screen for the anti-inflammatory activity of most NSAIDs, and is considered predictive of human efficacy. The anti-inflammatory activity of the test CD2 antagonists or pharmaceutical compositions of invention is expressed as the percent inhibition of the increase in hind paw weight of the test group relative to the vehicle dosed control group.

In a specific embodiment of the invention where the experimental animal model used is adjuvant-induced arthritis rat model, body weight can be measured relative to a control group to determine the anti-inflammatory activity of CD2 antagonists or pharmaceutical compositions of invention. In another embodiment, the efficacy of CD2 antagonists or pharmaceutical compositions of invention can be assessed using assays that determine bone loss. Animal models such as ovariectomy-induced bone resorption mice, rat and rabbit models are known in the art for obtaining dynamic parameters for bone formation. Using methods such as those described by Yositake et al. or Yamamoto et al., bone volume is measured in vivo by microcomputed tomography analysis and bone histomorphometry analysis. Yoshitake et al., “Osteopontin-Deficient Mice Are Resistant to Ovariectomy-Induced Bone Resorption,” Proc. Natl. Acad. Sci. 96:8156-8160, (1999); Yamamoto et al., “The Integrin Ligand Echistatin Prevents Bone Loss in Ovariectomized Mice and Rats,” Endocrinology 139(3):1411-1419, (1998), both incorporated herein by reference in their entirety.

Additionally, animal models for inflammatory bowel disease can also be used to assess the efficacy of the CD2 antagonists or pharmaceutical compositions of invention (Kim eta l., 1992, Scand. J. Gastroentrol. 27:529-537; Strober, 1985, Dig. Dis. Sci. 30(12 Suppl):3S-10S). Ulcerative cholitis and Crohn's disease are human inflammatory bowel diseases that can be induced in animals. Sulfated polysaccharides including, but not limited to amylopectin, carrageen, amylopectin sulfate, and dextran sulfate or chemical irritants including but not limited to trinitrobenzenesulphonic acid (TNBS) and acetic acid can be administered to animals orally to induce inflammatory bowel diseases.

Animal models for asthma can also be used to assess the efficacy of CD2 antagonists or pharmaceutical compositions of invention. An example of one such model is the murine adoptive transfer model in which aeroallergen provocation of TH 1 or TH2 recipient mice results in TH effector cell migration to the airways and is associated with an intense neutrophilic (THI) and eosinophilic (TH2) lung mucosal inflammatory response (Cohn et al., 1997, J. Exp. Med. 1861737-1747).

Animal models for autoimmune disorders can also be used to assess the efficacy of CD2 antagonists or pharmaceutical compositions of invention. Animal models for autoimmune disorders such as type 1 diabetes, thyroid autoimmunity, sytemic lupus eruthematosus, and glomerulonephritis have been developed (Flanders et al., 1999, Autoimmunity 29:235-246; Krogh et al., 1999, Biochimie 81:511-515; Foster, 1999, Semin. Nephrol. 19:12-24).

The efficacy of CD2 antagonists or pharmaceutical compositions of invention can also be tested in such autoimmune disorder models as an experimental allergic encephalomyelitis (EAE) model. EAE is an experimental autoimmune disease of the central nervous system (CNS) (Zamvil et al, 1990, Ann. Rev, Immunol. 8:579) and is a disease model for the human autoimmune condition, multiple sclerosis (MS). EAE is an example of a cell-mediated autoimmune disorder that is mediated via T cells. EAE is readily induced in mammalian species by immunizations of myelin basic protein (MBP) purified from the CNS or an encephalitogenic proteolipid (PLP). SJL/J mice are a susceptible strain of mice (H-2u) and, upon induction of EAE, these mice develop an acute paralytic disease and an acute cellular infiltrate is identifiable within the CNS. EAE spontaneously develops in MBP ₁₋₁₇ peptide-specific T cell receptor (TCR) transgenic mice (TgMBP⁺) of a RAG-1-deficient background (Lafaille et al., 1994, Cell 78:399).

Further, any assays known to those skilled in the art can be used to evaluate CD2 antagonists or the pharmaceutical compositions disclosed herein for autoimmune and/or inflammatory diseases.

The toxicity and/or efficacy of CD2 antagonists or pharmaceutical compositions of invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅/ED₅₀. CD2 antagonists that exhibit large therapeutic indices are preferred. While CD2 antagonists that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of CD2 antagonists for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Efficacy in preventing or treating an autoimmune disorder may be demonstrated, e.g., by detecting the ability of a CD2 antagonist or composition of the invention to reduce one or more symptoms of the autoimmune disorder, to reduce mean absolute lymphocyte counts, to decrease T cell activation, to decrease T cell proliferation, to reduce cytokine production, or to modulate one or more particular cytokine profiles. Efficacy in preventing or treating psoriasis may be demonstrated, e.g., by detecting the ability of a CD2 antagonist or composition of the invention to reduce one or more symptoms of psoriasis, to reduce mean absolute lymphocyte counts, to reduce cytokine production, to modulate one or more particular cytokine profiles, to decrease scaling, to decrease erythema, to decrease plaque elevation, to decrease T cell activation in the dermis or epidermis of an affected area, to decrease T cell infiltration to the dermis or epidermis of an affected area, to reduce PASI, to improve the physician's global assessment score, or to improve quality of life. Efficacy in preventing or treating an inflammatory disorder may be demonstrated, e.g., by detecting the ability of a CD2 antagonist to reduce one or more symptoms of the inflammatory disorder, to decrease T cell activation, to decrease T cell proliferation, to modulate one or more cytokine profiles, to reduce cytokine production, to reduce inflammation of a joint, organ or tissue or to improve quality of life.

Changes in inflammatory disease activity may be assessed through tender and swollen joint counts, patient and physician global scores for pain and disease activity, and the ESR/CRP. Progression of structural joint damage may be assessed by quantitative scoring of X-rays of hands, wrists, and feet (Sharp method). Changes in functional status in humans with inflammatory disorders may be evaluated using the Health Assessment Questionnaire (HAQ), and quality of life changes are assessed with the SF-36.

4.6. Methods of Monitoring Lymphocyte Counts and Percent Binding

The effect of one or more doses of one or more CD2 antagonists, in particular CD2 binding molecules, on peripheral blood lymphocyte counts can be monitored/assessed using standard techniques known to one of skill in the art. Peripheral blood lymphocytes counts in a mammal can be determined by, e.g., obtaining a sample of peripheral blood from said mammal, separating the lymphocytes from other components of peripheral blood such as plasma using, e.g., Ficoll-Hypaque (Pharmacia) gradient centrifugation, and counting the lymphocytes using trypan blue. Peripheral blood T-cell counts in mammal can be determined by, e.g., separating the lymphocytes from other components of peripheral blood such as plasma using, e.g., a use of Ficoll-Hypaque (Pharmacia) gradient centrifugation, labeling the T-cells with an antibody directed to a T-cell antigen such as CD3, CD4, and CD8 which is conjugated to FITC or phycoerythrin, and measuring the number of T-cells by FACS. Further, the effect on a particular subset of T cells (e.g., CD2⁺, CD4⁺, CD8⁺, CD4⁺RO⁺, CD8⁺RO⁺, CD4⁺RA⁺, or CD8⁺RA⁺) or NK cells can be determined using standard techniques known to one of skill in the art such as FACS.

The percentage of CD2 polypeptides expressed by peripheral blood lymphocytes bound by CD2 binding molecules prior or after, or both prior to and after the administration of one or more doses of CD2 binding molecules can be assessed using standard techniques known to one of skill in the art. The percentage of CD2 polypeptides expressed by peripheral blood T-cells bound by CD2 binding molecules can be determined by, e.g., obtaining a sample of peripheral blood from a mammal, separating the lymphocytes from other components of peripheral blood such as plasma using, e.g., Ficoll-Hypaque (Pharmacia) gradient centrifugation, and labeling the T-cells with an anti-CD2 binding molecule antibody conjugated to FITC and an antibody directed to a T-cell antigen such as CD3, CD4 or CD8 which is conjugated to phycoerythrin, and determining the number of T-cells labeled with anti-CD2 binding molecule antibody relative to the number of T-cells labeled with an antibody directed to a T-cell antigen using FACS. The percentage of CD2 polypeptides expressed by NK cells bound by CD2 binding molecules can also be assessed using standard techniques known to one of skill in the art, including, e.g., FACS.

4.7. Methods of Producing Antibodies

The antibodies that immunospecifically bind to a CD2 polypeptide can be produced by any method known in the art for the synthesis of antibodies, in particular, by chemical synthesis or preferably, by recombinant expression techniques.

Polyclonal antibodies immunospecific for a CD2 polypeptide can be produced by various procedures well known in the art. For example, a human CD2 polypeptide can be administered to various host animals including, but not limited to, rabbits, mice, rats, etc. to induce the production of sera containing polyclonal antibodies specific for the human CD2 polypeptide. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies. A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with a non-murine CD2 polypeptide and once an immune response is detected, e.g., antibodies specific for the CD2 polypeptide are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

Accordingly, the present invention provides methods of generating monoclonal antibodies as well as antibodies produced by the method comprising culturing a hybridoma cell secreting an antibody of the invention wherein, preferably, the hybridoma is generated by fusing splenocytes isolated from a mouse immunized with a non-murine CD2 polypeptide with myeloma cells and then screening the hybridomas resulting from the fusion for hybridoma clones that secrete an antibody able to bind a CD2 polypeptide.

Antibody fragments which recognize specific CD2 epitopes may be generated by any technique known to those of skill in the art. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain. Further, the antibodies of the present invention can also be generated using various phage display methods known in the art.

In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of lymphoid tissues). The DNA encoding the VH and VL domains are recombined together with an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to a CD2 polypeptide can be selected or identified with antigen, e.g., using labeled antigen or antigen bound or captured to a solid surface or bead. Examples of phage display methods that can be used to make the antibodies of the present invention include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; PCT application No. PCT/GB91/O1 134; PCT publication Nos. WO 90/02809, WO 91/10737, WO 92/01047, WO 92/18619, WO 93/1 1236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)2 fragments can also be employed using methods known in the art such as those disclosed in PCT publication No. WO 92/22324; Mullinax et al., 1992, BioTechniques 12(6):864-869; Sawai et al., 1995, AJRI 34:26-34; and Better et al., 1988, Science 240:1041-1043 (said references incorporated by reference in their entireties).

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g., human kappa or lamba constant regions. Preferably, the vectors for expressing the VH or VL domains comprise an EF-1α promoter, a secretion signal, a cloning site for the variable domain, constant domains, and a selection marker such as neomycin. The VH and VL domains may also cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

For some uses, including in vivo use of antibodies in humans and in vitro detection assays, it may be preferable to use human or chimeric antibodies. Completely human antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and PCT publications WO 98/46645, WO 98/50433, WO 98/24893, WO98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the J_{H} region prevents endogenous antibody production. The modified embryonic stem cells are expanded and microinjected into blastocysts to produce chimeric mice. The chimeric mice are then be bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., PCT publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, which are incorporated by reference herein in their entirety. In addition, companies such as Abgenix, Inc. (Freemont, Calif.) and Genpharm (San Jose, Calif.) can be engaged to provide human antibodies directed against a selected antigen using technology similar to that described above.

A chimeric antibody is a molecule in which different portions of the antibody are derived from different immunoglobulin molecules such as antibodies having a variable region derived from a human antibody and a non-human immunoglobulin constant region. Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, and 4,816,397, which are incorporated herein by reference in their entirety. Chimeric antibodies comprising one or more CDRs from human species and framework regions from a non-human immunoglobulin molecule can be produced using a variety of techniques known in the art including, for example, CDR-grafting (EP 239,400; PCT publication No. WO 91/09967; and U.S. Pat. Nos. 5,225,539, 5,530,101, and 5,585,089), veneering or resurfacing (EP 592,106; EP 519,596; Padlan, 1991, Molecular Immunology 28(4/5):489-498; Studnicka et al., 1994, Protein Engineering 7(6):805-814; and Roguska et al., 1994, PNAS 91:969-973), and chain shuffling (U.S. Pat. No. 5,565,332). In a preferred embodiment, chimeric antibodies comprise a human CDR3 having an amino acid sequence of any one of the CDR3 listed in Table 1 and non-human framework regions. Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature 332:323, which are incorporated herein by reference in their entireties.)

Further, the antibodies that immunospecifically bind to a CD2 polypeptide can, in turn, be utilized to generate anti-idiotype antibodies that “mimic” a CD2 polypeptide using techniques well known to those skilled in the art. (See, e.g., Greenspan & Bona, 1989, FASEB J. 7(5):437-444; and Nissinoff, 1991, J. Immunol. 147(8):2429-2438).

4.7.1. Polynucleotides Encoding Antibodies

The invention provides polynucleotides comprising a nucleotide sequence encoding an antibody that immunospecifically binds to a CD2 polypeptide. The invention also encompasses polynucleotides that hybridize under high stringency, intermediate or lower stringency hybridization conditions, e.g., as defined supra, to polynucleotides that encode an antibody of the invention.

The polynucleotides may be obtained, and the nucleotide sequence of the polynucleotides determined, by any method known in the art. The nucleotide sequence of antibodies immunospecific for a CD2 polypeptide can be obtained, e.g., from the literature or a database such as GenBank. Since the amino acid sequences of, e.g., LoCD2a/BTI-322, LO-CD2b and MEDI-507 are known, nucleotide sequences encoding these antibodies can be determined using methods well known in the art, i.e., nucleotide codons known to encode particular amino acids are assembled in such a way to generate a nucleic acid that encodes the antibody. Such a polynucleotide encoding the antibody may be assembled from chemically synthesized oligonucleotides (e.g., as described in Kutmeier et al., 1994, BioTechniques 17:242), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably poly A+ RNA, isolated from, any tissue or cells expressing the antibody, such as hybridoma cells selected to express an antibody of the invention) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence of the antibody is determined, the nucleotide sequence of the antibody may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties), to generate antibodies having a different amino acid sequence, for example to create amino acid substitutions, deletions, and/or insertions.

In a specific embodiment, one or more of the CDRs is inserted within framework regions using routine recombinant DNA techniques. The framework regions may be naturally occurring or consensus framework regions, and preferably human framework regions (see, e.g., Chothia et al., 1998, J. Mol. Biol. 278: 457-479 for a listing of human framework regions). Preferably, the polynucleotide generated by the combination of the framework regions and CDRs encodes an antibody that specifically binds to a CD2 polypeptide. Preferably, as discussed supra, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

4.7.2. Recombinant Expression of Antibodies

Recombinant expression of an antibody that immunospecifically binds to a CD2 polypeptide requires construction of an expression vector containing a polynucleotide that encodes the antibody. Once a polynucleotide encoding an antibody molecule of the invention has been obtained, the vector for the production of the antibody molecule may be produced by recombinant DNA technology using techniques well known in the art. See, e.g., U.S. Pat. No. 6,331,415, which is incorporated herein by reference in its entirety. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, a heavy or light chain of an antibody, a heavy or light chain variable domain of an antibody or a portion thereof, or a heavy or light chain CDR, operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464) and the variable domain of the antibody may be cloned into such a vector for expression of the entire heavy, the entire light chain, or both the entire heavy and light chains.

The expression vector is transferred to a host cell by conventional techniques and the transfected cells are then cultured by conventional techniques to produce an antibody of the invention. Thus, the invention includes host cells containing a polynucleotide encoding an antibody of the invention or fragments thereof, or a heavy or light chain thereof, or portion thereof or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules of the invention (see, e.g., U.S. Pat. No. 5,807,715). Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, NSO, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., 1986, Gene 45:101; and Cockett et al., 1990, Bio/Technology 8:2). In a specific embodiment, the expression of nucleotide sequences encoding antibodies which immunospecifically bind to one or more CD2 binding molecules is regulated by a constitutive promoter, inducible promoter or tissue specific promoter.

In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited to, the E. coli expression vector pUR278 (Ruther et al., 1983, EMBO 12:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, Proc. Natl. Acad. Sci. USA 8 1:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:51-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, WI 38, BT483, Hs578T, HTB2, BT2O and T47D, NSO (a murine myeloma cell line that does not endogenously produce any immunoglobulin chains), CRL7O3O and HsS78Bst cells.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compositions that interact directly or indirectly with the antibody molecule.

A number of selection systems may be used, including but not limited to, the herpes simplex virus thymidine kinase (Wigler et al., 1977, Cell 11:223), hypoxanthineguanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk-, hgprt- or aprt-cells, respectively. Also, antimetabolite resistance can be used as the basis of selection for the following genes: dhfr, which confers resistance to methotrexate (Wigler et al., 1980, Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62: 191-217; May, 1993, TIB TECH 11(5):155-2 15); and hygro, which confers resistance to hygromycin (Santerre et al., 1984, Gene 30:147). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., 1981, J. Mol. Biol. 150:1, which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol.3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, 1986, Nature 322:52; and Kohler, 1980, Proc. Natl. Acad. Sci. USA 77:2 197). The coding sequences for the heavy and light chains may comprise cDNA or genomic DNA.

Once an antibody molecule of the invention has been produced by recombinant expression, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies of the present invention or fragments thereof may be fused to heterologous polypeptide sequences described herein or otherwise known in the art to facilitate purification.

4.8. Methods For Producing Polypeptides and Fusion Proteins

Peptides, polypeptides, proteins and fusion proteins can be produced by standard recombinant DNA techniques or by protein synthetic techniques, e.g., by use of a peptide synthesizer. For example, a nucleic acid molecule encoding a polypeptide or a fusion protein can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, e.g., Current Protocols in Molecular Biology, Ausubel et al., eds., John Wiley & Sons, 1992). Moreover, a nucleic acid encoding a bioactive molecule can be cloned into an expression vector containing the Fc domain or a fragment thereof such that the bioactive molecule is linked in-frame to the Fc domain or Fc domain fragment.

Methods for fusing or conjugating polypeptides to the constant regions of antibodies are known in the art. See, e.g., U.S. Pat. Nos. 5,336,603, 5,622,929, 5,359,046, 5,349,053, 5,447,851, 5,723,125, 5,783,181, 5,908,626, 5,844,095, and 5,112,946; EP 307,434; EP 367,166; EP 394,827; PCT publications WO 91/06570, WO 96/04388, WO 96/22024, WO 97/34631, and WO 99/04813; Ashkenazi et al., 1991, Proc. Natl. Acad. Sci. USA 88: 10535-10539; Traunecker et al., 1988, Nature, 331:84-86; Zheng et al., 1995, J. Immunol. 154:5590-5600; and Vil et al., 1992, Proc. Natl. Acad. Sci. USA 89:11337-11341, which are incorporated herein by reference in their entireties.

The nucleotide sequences encoding a bioactive molecule and an Fc domain or fragment thereof may be an be obtained from any information available to those of skill in the art (i.e., from Genbank, the literature, or by routine cloning). The nucleotide sequence coding for a polypeptide a fusion protein can be inserted into an appropriate expression vector, i.e., a vector which contains the necessary elements for the transcription and translation of the inserted protein-coding sequence. A variety of host-vector systems may be utilized in the present invention to express the protein-coding sequence. These include but are not limited to mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

The expression of a polypeptide or a fusion protein may be controlled by any promoter or enhancer element known in the art. Promoters which may be used to control the expression of the gene encoding fusion protein include, but are not limited to, the SV40 early promoter region (Bemoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42), the tetracycline (Tet) promoter (Gossen et al., 1995, Proc. Nat. Acad. Sci. USA 89:5547-5551); prokaryotic expression vectors such as the β-lactamase promoter (Villa-Kamaroff, et al., 1978, Proc. Natl. Acad. Sci. U.S.A. 75:3727-3731), or the tac promoter (DeBoer, et al., 1983, Proc. Natl. Acad. Sci. U.S.A. 80:21-25; see also “Useful proteins from recombinant bacteria” in Scientific American, 1980, 242:74-94); plant expression vectors comprising the nopaline synthetase promoter region (Herrera-Estrella et al., Nature 303:209-213) or the cauliflower mosaic virus 35S RNA promoter (Gardner, et al., 1981, Nucl. Acids Res. 9:2871), and the promoter of the photosynthetic enzyme ribulose biphosphate carboxylase (Herrera-Estrella et al., 1984, Nature 310:115-120); promoter elements from yeast or other fungi such as the Gal 4 promoter, the ADC (alcohol dehydrogenase) promoter, PGK (phosphoglycerol kinase) promoter, alkaline phosphatase promoter, and the following animal transcriptional control regions, which exhibit tissue specificity and have been utilized in transgenic animals: elastase I gene control region which is active in pancreatic acinar cells (Swift et al., 1984, Cell 38:639-646; Omitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlaufet al., 1985, Mol. Cell. Biol. 5:1639-1648; Hammer et al., 1987, Science 235:53-58; alpha 1-antitrypsin gene control region which is active in the liver (Kelsey et al., 1987, Genes and Devel. 1:161-171), beta-globin gene control region which is active in myeloid cells (Mogram et al., 1985, Nature 315:338-340; Kollias et al., 1986, Cell 46:89-94; myelin basic protein gene control region which is active in oligodendrocyte cells in the brain (Readhead et al., 1987, Cell 48:703-712); myosin light chain-2 gene control region which is active in skeletal muscle (Sani, 1985, Nature 314:283-286); neuronal-specific enolase (NSE) which is active in neuronal cells (Morelli et al., 1999, Gen. Virol. 80:571-83); brain-derived neurotrophic factor (BDNF) gene control region which is active in neuronal cells (Tabuchi et al., 1998, Biochem. Biophysic. Res. Corn. 253:818-823); glial fibrillary acidic protein (GFAP) promoter which is active in astrocytes (Gomes et al., 1999, Braz J Med Biol Res 32(5):619-631; Morelli et al., 1999, Gen. Virol. 80:571-83) and gonadotropic releasing hormone gene control region which is active in the hypothalamus (Mason et al., 1986, Science 234:1372-1378).

In a specific embodiment, the expression of a polypeptide or a fusion protein is regulated by a constitutive promoter. In another embodiment, the expression of a polypeptide or a fusion protein is regulated by an inducible promoter. In another embodiment, the expression of a polypeptide or a fusion protein is regulated by a tissue-specific promoter.

In a specific embodiment, a vector is used that comprises a promoter operably linked to a polypeptide- or a fusion protein-encoding nucleic acid, one or more origins of replication, and, optionally, one or more selectable markers (e.g., an antibiotic resistance gene).

In mammalian host cells, a number of viral-based expression systems may be utilized. In cases where an adenovirus is used as an expression vector, the polypeptide or fusion protein coding sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, Proc. Natl. Acad. Sci. USA 81:355-359). Specific initiation signals may also be required for efficient translation of inserted fusion protein coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987, Methods in Enzymol. 153:51-544).

Expression vectors containing inserts of a gene encoding a polypeptide or a fusion protein can be identified by three general approaches: (a) nucleic acid hybridization, (b) presence or absence of “marker” gene functions, and (c) expression of inserted sequences. In the first approach, the presence of a gene encoding a polypeptide or a fusion protein in an expression vector can be detected by nucleic acid hybridization using probes comprising sequences that are homologous to an inserted gene encoding the polypeptide or the fusion protein, respectively. In the second approach, the recombinant vector/host system can be identified and selected based upon the presence or absence of certain “marker” gene functions (e.g., thymidine kinase activity, resistance to antibiotics, transformation phenotype, occlusion body formation in baculovirus, etc.) caused by the insertion of a nucleotide sequence encoding a polypeptide or a fusion protein in the vector. For example, if the nucleotide sequence encoding the fusion protein is inserted within the marker gene sequence of the vector, recombinants containing the gene encoding the fusion protein insert can be identified by the absence of the marker gene function. In the third approach, recombinant expression vectors can be identified by assaying the gene product (e.g., fusion protein) expressed by the recombinant. Such assays can be based, for example, on the physical or functional properties of the fusion protein in in vitro assay systems, e.g., binding with anti-bioactive molecule antibody.

In addition, a host cell strain may be chosen which modulates the expression of the inserted sequences, or modifies and processes the gene product in the specific fashion desired. Expression from certain promoters can be elevated in the presence of certain inducers; thus, expression of the genetically engineered fusion protein may be controlled. Furthermore, different host cells have characteristic and specific mechanisms for the translational and post-translational processing and modification (e.g., glycosylation, phosphorylation of proteins). Appropriate cell lines or host systems can be chosen to ensure the desired modification and processing of the foreign protein expressed. For example, expression in a bacterial system will produce an unglycosylated product and expression in yeast will produce a glycosylated product. Eukaryotic host cells which possess the cellular machinery for proper processing of the primary transcript, glycosylation, and phosphorylation of the gene product may be used. Such mammalian host cells include, but are not limited to, CHO, VERY, BHK, Hela, COS, MDCK, 293, 3T3, WI38, NSO, and in particular, neuronal cell lines such as, for example, SK-N-AS, SK-N-Fl, SK-N-DZ human neuroblastomas (Sugimoto et al., 1984, J. Natl. Cancer Inst. 73: 51-57), SK-N-SH human neuroblastoma (Biochim. Biophys. Acta, 1982, 704: 450-460), Daoy human cerebellar medulloblastoma (He et al., 1992, Cancer Res. 52: 1144-1148) DBTRG-05MG glioblastoma cells (Kruse et al., 1992, In Vitro Cell. Dev. Biol. 28A: 609-614), IMR-32 human neuroblastoma (Cancer Res., 1970, 30: 2110-2118), 1321N1 human astrocytoma (Proc. Natl Acad. Sci. USA, 1977, 74: 4816), MOG-G-CCM human astrocytoma (Br. J. Cancer, 1984, 49: 269), U87MG human glioblastoma-astrocytoma (Acta Pathol. Microbiol. Scand., 1968, 74: 465-486), A172 human glioblastoma (Olopade et al., 1992, Cancer Res. 52: 2523-2529), C6 rat glioma cells (Benda et al., 1968, Science 161: 370-371), Neuro-2a mouse neuroblastoma (Proc. Natl. Acad. Sci. USA, 1970, 65: 129-136), NB41A3 mouse neuroblastoma (Proc. Natl. Acad. Sci. USA, 1962, 48: 1184-1190), SCP sheep choroid plexus (Bolin et al., 1994, J. Virol. Methods 48: 211-221), G355-5, PG-4 Cat normal astrocyte (Haapala et al., 1985, J. Virol. 53: 827-833), Mpf ferret brain (Trowbridge et al., 1982, In Vitro 18: 952-960), and normal cell lines such as, for example, CTX TNA2 rat normal cortex brain (Radany et al., 1992, Proc. Natl. Acad. Sci. USA 89: 6467-6471) such as, for example, CRL7030 and Hs578Bst. Furthermore, different vector/host expression systems may effect processing reactions to different extents.

For long-term, high-yield production of recombinant proteins, stable expression is preferred. For example, cell lines which stably express a polypeptide or a fusion protein may be engineered. Rather than using expression vectors which contain viral origins of replication, host cells can be transformed with DNA controlled by appropriate expression control elements (e.g., promoter, enhancer, sequences, transcription termina-tors, polyadenylation sites, etc.), and a selectable marker. Following the introduction of the foreign DNA, engineered cells may be allowed to grow for 1-2 days in an enriched medium, and then are switched to a selective medium. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express a polypeptide or a fusion protein that immunospecifically binds to a CD2 polypeptide. Such engineered cell lines may be particularly useful in screening and evaluation of compounds that affect the activity of a polypeptide or a fusion protein that immunospecifically binds to a CD2 polypeptide.

A number of selection systems may be used, including but not limited to the herpes simplex virus thymidine kinase (Wigler, et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy, et al., 1980, Cell 22:817) genes can be employed in tk-, hgprt- or aprt-cells, respectively. Also, antimetabolite resistance can be used as the basis of selection for dhfr, which confers resistance to methotrexate (Wigler, et al., 1980, Natl. Acad. Sci. USA 77:3567; O'Hare, et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin, et al., 1981, J. Mol. Biol. 150:1); and hygro, which confers resistance to hygromycin (Santerre, et al., 1984, Gene 30:147) genes.

Once a polypeptide or a fusion protein of the invention has been produced by recombinant expression, it may be purified by any method known in the art for purification of a protein, for example, by chromatography (e.g., ion exchange, affinity, particularly by affinity for the specific antigen after Protein A, and sizing column chromatography), centrifugation, differential solubility, or by any other standard technique for the purification of proteins.

4.9. Articles of Manufacture

The present invention also encompasses a finished packaged and labeled pharmaceutical product. This article of manufacture includes the appropriate unit dosage form in an appropriate vessel or container such as a glass vial or other container that is hermetically sealed. In the case of dosage forms suitable for parenteral administration the active ingredient, e.g., the CD2 antagonist, is sterile and suitable for administration as a particulate free solution. In other words, the invention encompasses both parenteral solutions and lyophilized powders, each being sterile, and the latter being suitable for reconstitution prior to injection. Alternatively, the unit dosage form may be a solid suitable for oral, transdermal, topical or mucosal delivery.

In a preferred embodiment, the unit dosage form is suitable for intravenous, intramuscular, topical or subcutaneous delivery. Thus, the invention encompasses solutions, preferably sterile, suitable for each delivery route.

As with any pharmaceutical product, the packaging material and container are designed to protect the stability of the product during storage and shipment. Further, the products of the invention include instructions for use or other informational material that advise the physician, technician or patient on how to appropriately prevent or treat the disease or disorder in question. In other words, the article of manufacture includes instruction means indicating or suggesting a dosing regimen including, but not limited to, actual doses, monitoring procedures, total lymphocyte and T-cell counts and other monitoring information.

Specifically, the invention provides an article of manufacture comprising packaging material, such as a box, bottle, tube, vial, container, sprayer, insufflator, intravenous (i.v.) bag, envelope and the like; and at least one unit dosage form of a pharmaceutical agent contained within said packaging material, wherein said pharmaceutical agent comprises a CD2 antagonist and wherein said packaging material includes instruction means which indicate that said CD2 antagonist can be used to treat, prevent or impede the symptoms of autoimmune disease or inflammatory disorder by administering specific doses and using specific dosing regimens as described herein in order to achieve the lymphocyte or T-cell counts as described herein. More specifically, the invention provides an article of manufacture comprising packaging material, such as a box, bottle, tube, vial, container, sprayer, insufflator, intravenous (i.v.) bag, envelope and the like; and at least one unit dosage form of a pharmaceutical agent contained within said packaging material, wherein said pharmaceutical agent comprises a CD2 binding molecule and wherein said packaging material includes instruction means which indicate that said CD2 binding molecule can be used to treat, prevent or impede the symptoms of autoimmune disease or inflammatory disorder by administering specific doses and using specific dosing regimens as described herein in order to achieve the lymphocyte or T-cell counts as described herein. In a preferred embodiment, the instruction means indicate or suggest that lymphocyte or T-cell counts be monitored one or more times before and/or after a dose. For example, the instruction means can indicate that a lymphocyte count be taken before the first dose and after one or more subsequent doses. In a specific embodiment the instruction means indicate that the CD2 binding molecule is to be used to treat chronic plaque psoriasis and that the lymphocyte count should be reduced to below 1200 cells/mm³ (preferably below 1000 cells/mm³) after the administration and not below 500 cells/mm³ (preferably 750 cells/mm³) for more than a short period of time. Finally, the instruction means in another embodiment will indicate the desired percentage of binding of the CD2 binding molecule to CD2 polypeptides expressed by peripheral blood lymphocytes (preferably, peripheral blood T-cells, a subset of peripheral blood T-cells, and/or NK cells), the desired percent reduction in lymphocyte count (preferably, peripheral blood T-cells, a subset of peripheral blood T-cells, and/or NK cells) after administration, and/or a means for determining the PASI score. Suitable instruction means include printed labels, printed package inserts, tags, cassette tapes, and the like.

In specific embodiment, an article of manufacture comprises packaging material and an injectable form of a pharmaceutical agent contained within said packaging material, wherein said pharmaceutical agent comprises a CD2 binding molecule and a pharmaceutically acceptable carrier, wherein said article of manufacture includes instruction means indicating a dosing regimen comprising administering an initial dosing, and optionally administering a subsequent dose or doses, of said pharmaceutical agent to a patient suffering from one or more symptoms associated with an autoimmune disorder characterized by increased infiltration of activated T-cells into affected tissues or fluids, wherein the instruction means suggests a dosing regimen comprising an initial dosing that results in CD2 binding molecules binding to at least 25%, preferably at least 30%, at least 35%, at least 40%, at least 45%, at least 50%, at least 55%, at least 60%, at least 70%, at least 75%, at least 80%, at least 85% or at least 90% of the CD2 polypeptides expressed by the patient's peripheral blood lymphocytes for at least 30 minutes, 1 hour, 2 hours, 4 hours, 6 hours, 8 hours, 10 hours, 12 hours, 16 hours, 24 hours, 48 hours, 72 hours, 1 week or more after the administration of said initial dosing, and wherein the instruction means suggests a dosing interval for said dosing regimen such that any dose/doses administered subsequent to said initial dosing, if administered, is/are only administered when 20% or less, preferably 15% or less or 10% or less of the CD2 polypeptides expressed by peripheral blood lymphocyes are bound by previously administered CD2 binding molecules.

In another embodiment, an article of manufacture comprising packaging material and a pharmaceutical agent contained within said packaging material, wherein said pharmaceutical agent comprises a CD2 binding molecule and a pharmaceutically acceptable carrier, wherein said article of manufacture includes instruction means indicating a dosing regimen comprising administering an initial dosing, and optionally administering a subsequent dose or doses, of said pharmaceutical agent to a subject suffering from one or more symptoms associated with an autoimmune disorder or an inflammatory disorder, wherein the instruction means suggests a dosing regimen comprising an initial dosing that results in CD2 binding molecules binding to 30% to 90% of the CD2 molecules expressed by the subject's peripheral blood lymphocytes for at least 1 hour after the administration of said initial dosing, and wherein the instruction means suggests a dosing interval for said dosing regimen such that any dose/doses administered subsequent to said initial dosing, if administered, is/are only administered when 25% or less of the CD2 polypeptides expressed by peripheral blood lymphocytes are bound by previously administered CD2 binding molecules.

In another embodiment, an article of manufacture comprising packaging material and a pharmaceutical agent contained within said packaging material, wherein said pharmaceutical agent comprises a CD2 binding molecule and a pharmaceutically acceptable carrier, wherein said article of manufacture includes instruction means indicating a dosing regimen comprising administering an initial dosing, and optionally administering a subsequent dose or doses, of said pharmaceutical agent to a subject suffering from one or more symptoms associated with an autoimmune disorder or an inflammatory disorder, wherein the instruction means suggests a dosing regimen comprising an initial dosing that results in a mean absolute lymphocyte count of below 1250 cells/mm³ (preferably, below 1000 cells/mm³) but not below 500 cells/mm³, and wherein the instruction means suggests a dosing interval for said dosing regimen such that any dose/doses administered subsequent to said initial dosing, if administered, is/are only administered when the mean absolute lymphocyte count is increases above a particular number such as, e.g., 1250 cells/mm³, preferably 1500 cells/mm³.

In a preferred embodiment, an article of manufacture comprising packaging material and a pharmaceutical composition in suitable form for administration to a human contained within said packaging material, wherein said pharmaceutical composition comprises MEDI-507 or an antigen-binding fragment thereof, and a pharmaceutically acceptable carrier. In accordance with the invention, such an article of manufacture may further comprise instructions as described above.

The present invention provides that the adverse effects that may be reduced or avoided by the methods of the invention are indicated in informational material enclosed in an article of manufacture for use in preventing, treating or ameliorating one or more symptoms of an autoimmune disorder or an inflammatory disorder. Adverse effects that may be reduced or avoided by the methods of the invention include but are not limited to vital sign abnormalities (fever, tachycardia, bardycardia, hypertension, hypotension), hematological events (anemia, lymphopenia, leukopenia, thrombocytopenia), headache, chills, dizziness, nausea, asthenia, back pain, chest pain (chest pressure), diarrhea, myalgia, pain, pruritus, psoriasis, rhinitis, sweating, injection site reaction, and vasodilatation. Since CD2 antagonists, in particular, CD2 binding molecules may be immunosuppressive, prolonged immunosuppression may increase the risk of infection, including opportunistic infections. Prolonged and sustained immunosuppression may also result in an increased risk of developing certain types of cancer. Further, the information material enclosed in an article of manufacture for use in preventing, treating or ameliorating one or more symptoms of an autoimmune disorder can indicate that foreign proteins may also result in allergic reactions, including anaphylaxis, or cytosine release syndrome. The information material should indicate that allergic reactions may exhibit only as mild pruritic rashes or they may be severe such as erythroderma, Stevens-Johnson syndrome, vasculitis, or anaphylaxis. The information material should also indicate that anaphylactic reactions (anaphylaxis) are serious and occasionally fatal hypersensitivity reactions. Allergic reactions including anaphylaxis may occur when any foreign protein is injected into the body. They may range from mild manifestations such as urticaria or rash to lethal systemic reactions. Anaphylactic reactions occur soon after exposure, usually within 10 minutes. Patients may experience paresthesia, hypotension, laryngeal edema, mental status changes, facial or pharyngeal angioedema, airway obstruction, bronchospasm, urticaria and pruritus, serum sickness, arthritis, allergic nephritis, glomerulonephritis, temporal arthritis, or eosinophilia.

The information material can also indicate that cytokine release syndrome is an acute clinical syndrome, temporally associated with the administration of certain activating anti-T cell antibodies. Cytokine release syndrome has been attributed to the release of cytokines by activated lymphocytes or monocytes. The clinical manifestations for cytokine release syndrome have ranged from a more frequently reported mild, self-limited, “flu-like” illness to a less frequently reported severe, life-threatening, shock-like reaction, which may include serious cardiovascular, pulmonary and central nervous system manifestations. The syndrome typically begins approximately 30 to 60 minutes after administration (but may occur later) and may persist for several hours. The frequency and severity of this symptom complex is usually greatest with the first dose. With each successive dose, both the incidence and severity of the syndrome tend to diminish. Increasing the amount of a dose or resuming treatment after a hiatus may result in a reappearance of the syndrome. As mentioned above, the invention encompasses methods of treatment and prevention that avoid or reduce one or more of the adverse effects discussed herein.

Equivalents

Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference.

What is claimed is:

1. A method of treating or ameliorating an autoimmune disorder or an inflammatory disorder or one or more symptoms thereof, said method comprising administering to a subject in need thereof a dose of a therapeutically effective amount of one or more CD2 binding molecules, wherein administration of said dose results in CD2 binding molecules binding to at least 25% of the CD2 polypeptides expressed by peripheral blood lymphocytes.

2. A method of treating or ameliorating an autoimmune disorder or an inflammatory disorder or one or more symptoms thereof, said method comprising administering to a subject in need thereof a dose of a therapeutically effective amount of one or more CD2 binding molecules, wherein administration of said dose results in a mean absolute lymphocyte count of approximately 500 cells/μl to below 1200 cells/μl.

3. A method of treating or ameliorating an autoimmune disorder or an inflammatory disorder or one or more symptoms thereof, said method comprising administering to a subject in need thereof a dose of a therapeutically effective amount of one or more CD2 binding molecules, wherein administration of said dose results in an approximately 25% or more reduction in said subject's mean absolute lymphocyte count relative to said subject's mean absolute lymphocyte count prior to the administration of said dose.

4. The method of claim 2 further comprising administering to said subject one or more subsequent doses of a therapeutically effective amount of one or more CD2 binding molecules after administration of said first dose, wherein administration of said subsequent doses maintain a mean absolute lymphocyte count of approximately 500 cells/μl to below 1200 cells/μl.

5. The method of claim 2 further comprising administering to said subject one or more subsequent doses of a therapeutically effective amount of one or more CD2 binding molecules after administration of said first dose, wherein administration of said subsequent doses maintain an approximately 25% or more reduction in said subject's absolute mean lymphocyte count relative to said subject's mean absolute lymphocyte count prior to the administration of said first dose.

6. The method of claim 4, wherein said subsequent dose is administered at least 1 week after the administration of said first dose.

7. The method of claim 1 further comprising administering to said subject one or more subsequent doses of a therapeutically effective amount of one or more CD2 binding molecules after administration of said first dose, wherein said administration of said subsequent doses restore at least 25% of the CD2 polypeptides expressed by peripheral blood lymphocytes being bound by CD2 binding molecules.

8. The method of claim 1 or 3, wherein said dose results in CD2 binding molecules binding to at least 30%, at least 35%, at least 40%, at least 45%, at least 50%, at least 55%, at least 60%, at least 65%, at least 70%, at least 75% or at least 80% of the CD2 polypeptides expressed by peripheral blood lymphocytes for at least 1 hour after the administration of said dose and prior to the administration of a subsequent dose.

9. The method of claim 7, wherein said first dose results in CD2 binding molecules binding to at least 30%, at least 35%, at least 40%, at least 45%, at least 50%, at least 55%, at least 60%, at least 65%, at least 70%, at least 75% or at least 80% of the CD2 polypeptides expressed by peripheral blood lymphocytes.

10. The method of claim 7, wherein said first dose results in a mean absolute lymphocyte count of approximately 500 cells/μl to below 1200 cells/μl and said subsequent doses restore a mean absolute lymphocyte count of approximately 500 cells/μl to below 1200 cells/μl.

11. The method of claim 3 or 9, wherein a subsequent dose is administered when the percentage of CD2 polypeptides bound to CD2 binding molecules drops to 20% or less, 15% or less, or 10% or less.

12. The method of claim 3 or 9, wherein a subsequent dose is administered when the mean absolute lymphocyte count increases to approximately 1250 cells/μl or more.

13. A method of treating or ameliorating an autoimmune disorder or an inflammatory disorder or one or more symptoms thereof said method comprising administering to a subject in need thereof a dose of a therapeutically effective amount of one or more CD2 binding molecules and administering to said subject one or more subsequent doses of a therapeutically effective amount of one or more CD2 binding molecules after administering a prior dose, wherein said CD2 binding molecules do not inhibit the interaction between LFA-3 and CD2.

14. The method of claim 1, 2 or 3, wherein said CD2 binding molecules are not small organic molecules.

15. A method of treating or ameliorating an autoimmune disorder or an inflammatory disorder or one or more symptoms thereof, said method comprising:
(a) administering to a subject in need thereof one or more doses of a therapeutically effective amount of one or more CD2 binding molecules;
(b) monitoring the mean absolute lymphocyte count in said subject after the administration of one or more of said doses and prior to the administration of a subsequent dose; and
(c) maintaining a mean absolute lymphocyte count of approximately 500 cells/μl to below 1200 cells/μl by repeating step (a) as necessary.

16. A method of treating or ameliorating an autoimmune disorder or an inflammatory disorder or one or more symptoms thereof, said method comprising:
(a) administering to a subject in need thereof one or more doses of a therapeutically effective amount of one or more CD2 binding molecules;
(b) monitoring the mean absolute lymphocyte count of said subject after the administration of one or more of said doses and prior to the administration of a subsequent dose; and
(c) maintaining a mean absolute lymphocyte count in said subject of 25% less than the mean absolute lymphocyte count in said subject prior to the administration of said doses of therapeutically effective amounts of one or more CD2 binding molecules by repeating step (a) as necessary.

17. A method of treating or ameliorating an autoimmune disorder or an inflammatory disorder or one or more symptoms thereof, said method comprising:
(a) administering to a subject in need thereof one or more doses of a therapeutically effective amount of one or more CD2 binding molecules; and
(b) monitoring the mean absolute lymphocyte count in said subject after administration of a certain number of doses and prior to the administration of a subsequent dose.

18. The method of claim 17, wherein the certain number of doses is 1, 2, 3, 4, 5, 6,7,8,9, 10, 11 or 12 doses.

19. The method of claim 18 further comprising administering one or more subsequent doses of a therapeutically effective amount of one or more CD2 binding molecules based upon whether the lymphocyte count is within the range of approximately 500 cells/μl to 1200 cells/μl.

20. A method of treating or ameliorating an autoimmune disorder or an inflammatory disorder or one or more symptoms thereof, said method comprising:
(a) administering to a subject in need thereof one or more doses of a therapeutically effective amount of one or more CD2 binding molecules;
(b) assessing the percentage of CD2 polypeptides bound by CD2 binding molecules after administration of one or more of said doses and prior to the administration of a subsequent dose; and
(c) administering to said subject one or more subsequent doses of a therapeutically effective amount of one or more CD2 binding molecules when the percentage of CD2 polypeptides expressed by peripheral blood lymphocytes bound by CD2 binding molecules is approximately 20% or less.

21. A method of treating or ameliorating an autoimmune disorder or an inflammatory disorder or one or more symptoms thereof, said method comprising:
(a) administering to a subject in need thereof one or more doses of a therapeutically effective amount of one or more CD2 binding molecules;
(b) monitoring the percentage of CD2 polypeptides bound by CD2 binding molecules after administration of one or more of said doses and prior to the administration of a subsequent dose; and
(c) maintaining at least a 25% receptor occupancy by said CD2 binding molecules in said subject by repeating step (a) as necessary.

22. A method of treating or ameliorating an autoimmune disorder or an inflammatory disorder or one or more symptoms thereof, said method comprising:
(a) administering to a subject in need thereof one or more doses of a therapeutically effective amount of one or more CD2 binding molecules; and
(b) monitoring the percentage of CD2 polypeptides expressed by peripheral blood lymphocytes bound by CD2 binding molecules in said subject after administration of a certain number of doses and prior to the administration of a subsequent dose.

23. The method of claim 22, wherein the certain number of doses is 1, 2, 3, 4, 5, 6, 7, 8, 9, 10, 11 or 12 doses.

24. A method of treating or ameliorating psoriasis or one or more symptoms thereof, said method comprising administering to a subject in need thereof one or more doses of a therapeutically effective amount of one or more CD2 binding molecules, wherein administration of said doses results in a mean absolute lymphocyte count of approximately 500 cells/μl to below 1200 cells/μl.

25. A method of treating or ameliorating psoriasis or one or more symptoms thereof, said method comprising administering to a subject in need thereof one or more doses of a therapeutically effective amount of one or more CD2 binding molecules, wherein administration of said doses results in at least 25% of CD2 polypeptides expressed by peripheral blood lymphocytes being bound by CD2 binding molecules.

26. The method of claim 25, wherein said doses result in CD2 binding molecules binding to at least 30%, at least 35%, at least 40%, at least 45%, at least 50%, at least 55%, at least 60%, at least 65%, at least 70%, at least 75% or at least 80% of the CD2 polypeptides expressed by peripheral blood lymphocytes.

27. A method of treating or ameliorating psoriasis or one or more symptoms thereof, said method comprising administering to a subject in need thereof one or more doses of a therapeutically effective amount of MEDI-507.

28. The method of claim 27, wherein administration of said doses results in a lymphocyte count of approximately 500 cells/μl to below 1200 cells/μl.

29. The method of claim 27, wherein administration of said doses results in at least 30% of CD2 polypeptides expressed by peripheral blood lymphocytes being bound by MEDI-507.

30. The method of claim 29, wherein said doses result in at least 35%, at least 40%, at least 45%, at least 50%, at least 55%, at least 60%, at least 65%, at least 70%, at least 75% or at least 80% of the CD2 polypeptides expressed by peripheral blood lymphocytes being bound by MEDI-507.

31. The method of claim 24, 25, 26, 27 or 28, wherein at least a 25% reduction of said subject's Psoriasis Area and Severity Index (PASI) score is achieved.

32. The method of claim 31, wherein the PASI score is reduced by at least 50%.

33. The method of claim 31, wherein the PASI score is reduced by at least 75%.

34. A method of treating or ameliorating psoriasis in a human which method reduces or avoids adverse effects associated with decreasing lymphocyte counts, said method comprising administering doses of a therapeutically effective amount of one or more CD2 binding molecules, said doses being effective to achieve a reduction in said human's PASI score by at least 25% but insufficient to cause a reduction in lymphocyte count to below 500 cells/μl.

35. The method of claim 34, wherein at least a 50% reduction in said human's PASI score is achieved.

36. The method of claim 34, wherein the lymphocyte count is between 500 cells/μl and 1200 cells/μl.

37. The method as in any one of claims 1-5, 13, 17 and 22, wherein the autoimmune disorder is rheumatoid arthritis, psoriatic arthritis, ankylosing spondylitis, Reiter's Syndrome, systemic lupus erythematosus, dermatomyositis, Sjögren's syndrome, lupus erythematosus, multiple sclerosis, or myasthenia gravis.

38. The method as in any one of claims 1-5, 13, 17 and 22, wherein the inflammatory disorder is asthma, encephilitis, inflammatory bowel disease, chronic obstructive pulmonary disease (COPD), arthritis, or an allergic disorder.

39. The method as in any one of claims 1-5, 13, 17 and 22, wherein the autoimmune disorder is characterized by increased infiltration of lymphocytes into affected dermal or epidermal tissues.

40. The method as in any one of claims 1-5, 13, 17 and 22, wherein the autoimmune disorder is characterized by increased T cell activation or abnormal antigen presentation.

41. The method as in any one of claims 1-5, 13, 17 and 22, wherein the autoimmune disorder is psoriasis.

42. The method of claim 41, wherein the psoriasis is chronic plaque psoriasis.

43. The method of claim 1, 2, 3 or 13, wherein said dose is administered parenterally.

44. The method of claim 27, 28 or 29, wherein said doses of MEDI-507 are administered parenterally.

45. The method as in any one of claims 1-5, 13, 17, 22, and 24, wherein at least one of the CD2 binding molecules is a fusion protein.

46. The method as in any one of claims 1-5, 13, 17, 22, and 24, wherein at least one of the CD2 binding molecules is an antibody.

47. The method of claim 46, wherein the antibody is LO-CD2a/BTI-322 or an antigen-binding fragment thereof.

48. The method of claim 46, wherein the antibody is a human or humanized monoclonal antibody.

49. The method of claim 46, wherein the antibody is MEDI-507 or an antigen-binding fragment thereof.

50. The method as in any one of claims 1-5, 13, 17, 22, 24, and 27-29, wherein said effective amount is a dose of approximately 150 μg/kg or less, approximately 125 μg/kg or less, approximately 100 μg/kg or less, approximately 75 μg/kg or less, approximately 50 μg/kg or less, approximately 40 μg/kg or less, approximately 30 μg/kg or less, approximately 20 μg/kg or less, approximately 10 μg/kg or less, approximately 5 μg/kg or less, approximately 2 μg/kg or less, approximately 1 μg/kg or less, or approximately 0.5 μg/kg or less.

51. The method as in any one of claims 1-5, 13, 17, 22, 24, and 27-29, wherein said effective amount is a dose of between approximately 0.05 μg/kg and 150 μg/kg.

52. The method as in any one of claims 1-5, 13, 17, 22, 24, and 27-29, wherein the subject is a human subject.

53. The method of claim 4, 5, or 7, wherein the first dose is administered parenterally.

54. The method of claim 4, 5, or 7, wherein the subsequent doses are administered parenterally.

55. The method of claim 1, 2, 3 or 13, wherein said dose is administered subcutaneously.

56. The method of claim 27, 28 or 29, wherein said doses of MEDI-507 are administered subcutaneously.

57. The method of claim 4, 5, or 7, wherein the first dose is administered subcutaneously.

58. The method of claim 4, 5, or 7, wherein the subsequent doses are administered subcutaneously.

59. An article of manufacture comprising packaging material and a pharmaceutical agent contained within said packaging material,
wherein said pharmaceutical agent comprises a CD2 binding molecule and a pharmaceutically acceptable carrier,
wherein said article of manufacture includes instruction means indicating a dosing regimen comprising administering an initial dosing, and optionally administering a subsequent dose or doses, of said pharmaceutical agent to a subject suffering from one or more symptoms associated with an autoimmune disorder or an inflammatory disorder,
wherein the instruction means suggests a dosing regimen comprising an initial dosing that results in CD2 binding molecules binding to at least 30% of the CD2 polypeptides expressed by the subject's peripheral blood lymphocytes for at least 1 hour after the administration of said initial dosing, and
wherein the instruction means suggests a dosing interval for said dosing regimen such that any dose/doses administered subsequent to said initial dosing, if administered, is/are only administered when 20% or less of the CD2 polypeptides expressed by peripheral blood lymphocytes are bound by previously administered CD2 binding molecules.

60. An article of manufacture comprising packaging material and a pharmaceutical composition in suitable form for administration to a human contained within said packaging material, wherein said pharmaceutical composition comprises MEDI-507 or an antigen-binding fragment thereof, and a pharmaceutically acceptable carrier.


SEQUENCE LISTING (7 sequences)

SEQ ID NO: 1 - protein, 5 aa, Mus sp.
EYYMY

SEQ ID NO: 2 - protein, 17 aa, Mus sp.
RIDPEDGSIDYVEKFKK

SEQ ID NO: 3 - protein, 9 aa, Mus sp.
GKFNYRFAY

SEQ ID NO: 4 - protein, 16 aa, Mus sp.
RSSQSLLHSSGNTLNW

SEQ ID NO: 5 - protein, 7 aa, Mus sp.
LVSKLES

SEQ ID NO: 6 - protein, 9 aa, Mus sp.
MQFTHYPYT

SEQ ID NO: 7 - protein, 347 aa, Homo sapiens
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK